SEQUENCE LISTING (13 sequences)

SEQ ID NO: 1 - protein, 480 aa, Bacillus sp.
DGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGDVVMNHKMGADFTEAVQAVQVNPTNRWQDISGAYTIDAWTGFDFSGRNNAYSDFKWRWFHFNGVDWDQRYQENHIFRFANTNWNWRVDEENGNYDYLLGSNIDFSHPEVQDELKDWGSWFTDELDLDGYRLDAIKHIPFWYTSDWVRHQRNEADQDLFVVGEYWKDDVGALEFYLDEMNWEMSLFDVPLNYNFYRASQQGGSYDMRNILRGSLVEAHPMHAVTFVDNHDTQPGESLESWVADWFKPLAYATILTREGGYPNVFYGDYYGIPNDNISAKKDMIDELLDARQNYAYGTQHDYFDHWDVVGWTREGSSSRPNSGLATIMSNGPGGSKWMYVGRQNAGQTWTDLTGNNGASVTINGDGWGEFFTNGGSVSVYVNQ

SEQ ID NO: 2 - protein, 492 aa, Bacillus circulans
KRNHTMMQFFEWHLAADGDHWKRLAEMAPELKAKGIDTVWVPPVTKAVSAEDTGYGVYDLYDLGEFDQKGTVRTKYGTKQELIEAIAECQKNGIAVYVDLVMNHKAGADETEVFKVIEVDPNDRTKEISEPFEIEGWTKFTFPGRGDQYSSFKWNSEHFNGTDFDAREERTGVFRIAGENKKWNENVDDEFGNYDYLMFANIDYNHPDVRREMIDWGKWLIDTLQCGGFRLDAIKHINHEFIKEFAAEMIRKRGQDFYIVGEFWNSNLDACREFLDTVDYQIDLFDVSLHYKLHEASLKGRDFDLSKIFDDTLVQTHPTHAVTFVDNHDSQPHEALESWIGDWFKPSAYALTLLRRDGYPVVFYGDYYGIGGPEPVDGKKEILDILLSARCNKAYGEQEDYFDHANTIGWVRRGVEEIEGSGCAVVISNGDDGEKRMFIGEHRAGEVWVDLTKSCDDQITIEEDGWATFHVCGGGVSVWALPEQNEDCADAE

SEQ ID NO: 3 - protein, 485 aa, Bacillus sp.
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 4 - protein, 485 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 5 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 6 - protein, 485 aa, Bacillus sp.
HHNGTNGTMMQYFEWYLPNDGNHWNRLRDDAANLKSKGITAVWIPPAWKGTSQNDVGYGAYDLYDLGEFNQKGTVRTKYGTRNQLQAAVTSLKNNGIQVYGDVVMNHKGGADGTEIVNAVEVNRSNRNQETSGEYAIEAWTKFDFPGRGNNHSSFKWRWYHFDGTDWDQSRQLQNKIYKFRGTGKAWDWEVDTENGNYDYLMYADVDMDHPEVIHELRNWGVWYTNTLNLDGFRIDAVKHIKYSFTRDWLTHVRNTTGKPMFAVAEFWKNDLGAIENYLNKTSWNHSVFDVPLHYNLYNASNSGGYYDMRNILNGSVVQKHPTHAVTFVDNHDSQPGEALESFVQQWFKPLAYALVLTREQGYPSVFYGDYYGIPTHGVPAMKSKIDPLLQARQTFAYGTQHDYFDHHDIIGWTREGNSSHPNSGLATIMSDGPGGNKWMYVGKNKAGQVWRDITGNRTGTVTINADGWGNFSVNGGSVSVWVKQ

SEQ ID NO: 7 - protein, 483 aa, B. amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 8 - protein, 514 aa, B. stearothermophilus
APFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 9 - protein, 485 aa, Bacillus sp.
HHNGTNGTMMQYFEWHLPNDGNHWNRLRDDAANLKSKGITAVWIPPAWKGTSQNDVGYGAYDLYDLGEFNQKGTVRTKYGTRSQLQGAVTSLKNNGIQVYGDVVMNHKGGADGTEMVNAVEVNRSNRNQEISGEYTIEAWTKFDFPGRGNTHSNFKWRWYHFDGTDWDQSRQLQNKIYKFRGTGKAWDWEVDIENGNYDYLMYADIDMDHPEVINELRNWGVWYTNTLNLDGFRIDAVKHIKYSYTRDWLTHVRNTTGKPMFAVAEFWKNDLAAIENYLNKTSWNHSVFDVPLHYNLYNASNSGGYFDMRNILNGSVVQKHPIHAVTFVDNHDSQPGEALESFVQSWFKPLAYALILTREQGYPSVFYGDYYGIPTHGVPSMKSKIDPLLQARQTYAYGTQHDYFDHHDIIGWTREGDSSHPNSGLATIMSDGPGGNKWMYVGKHKAGQVWRDITGNRSGTVTINADGWGNFTVNGGAVSVWVKQ

SEQ ID NO: 10 - protein, 483 aa, Bacillus licheniformis [sequence omitted]

SEQ ID NO: 11 - protein, 485 aa, Bacillus sp.
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 12 - protein, 484 aa, Bacillus flavothermus
GSVPVNGTMMQYFEWYLPDDGTLWTKVANNAQSLANLGITALWLPPAYKGTSSSDVGYGVYDLYDLGEFNQKGTVRTKYGTKTQYIQAIQAAHTAGMQVYADVVFNHKAGADGTELVDAVEVNPSDRNQEISGTYQIQAWTKFDFPGRGNTYSSFKWRWYHFDGTDWDESRKLNRIYKFRGTGKAWDWEVDTENGNYDYLMYADLDMDHPEVVSELKNWGKWYVTTTNIDGFRLDAVKHIKYSFFPDWLSYVRTQTQKPLFAVGEFWSYDISKLHNYITKTNGSMSLFDAPLHNNFYIASKSGGYFDMRTLLNNTLMKDQPTLAVTLVDNHDTEPGQSLQSWVEPWFKPLAYAFILTRQEGYPCVFYGDYYGIPKYNIPALKSKLDPLLIARRDYAYGTQHDYIDSADIIGWTREGVAEKANSGLAALITDGPGGSKWMYVGKQHAGKTFYDLTGNRSDTVTINADGWGEFKVNGGSVSIWVPK

SEQ ID NO: 13 - protein, 485 aa, Artificial Sequence
  synthetic construct
ANTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

(19) United States
(12) Patent Application Publication
(10) Pub. No.: US 2014/0134709 A1
(43) Pub. Date: May 15, 2014

(54) Use of Amylase Variants at Low Temperature

(71) Applicant: NOVOZYMES A/S, BAGSVAERD (DK)

(72) Inventors: Carsten Andersen, Vaerloese (DK); Svend Kaasgaard, Skovlunde (DK); Lars Beier, Lyngby (DK); Jens Obro, Copenhagen (DK)

(73) Assignee: NOVOZYMES A/S, BAGSVAERD (DK)

(21) Appl. No.: 14/159,610

(22) Filed: Jan. 21, 2014

Related U.S. Application Data
(63) Continuation of application No. 13/516,566, filed on Nov. 26, 2012.
(60) Provisional application No. 61/289,481, filed on Dec. 23, 2009.

(30) Foreign Application Priority Data
Dec. 22, 2009 (EP) 09180439.3

(51) Int. Cl. C11D 3/386; C12N 9/26

(52) U.S. Cl. CPC C11D 3/38618; C12N 9/2414
USPC 435/264; 510/392; 206/223

(57) ABSTRACT

The present invention relates to the use of alpha-amylase variants having improved activity relative to the parent enzyme at low temperature, including improved washing and/or dishwashing performance and/or increased stability at low temperature. The invention further relates a method for doing laundry, dish wash and/or cleaning such as institutional cleaning and to compositions for use at low temperature.

### CROSS-REFERENCE TO RELATED APPLICATIONS

This application is a continuation of U.S. patent application Ser. No. 13/516,566 filed Nov. 26, 2012, which is a 35 U.S.C. 371 national application of PCT/EP2010/070586 filed Dec. 22, 2010, which claims priority or the benefit under 35 U.S.C. 119 of European application no. 09180439.3 filed Dec. 22, 2009 and U.S. provisional application No. 61/289,481 filed Dec. 23, 2009, the contents of which are fully incorporated herein by reference.

### FIELD OF THE INVENTION

The present invention relates to the use of alpha-amylase variants having improved activity relative to the parent enzyme at low temperature, including improved washing and/or dishwashing performance and/or increased stability at low temperature. The invention further relates to a method for cleaning such as e.g. doing laundry, dish wash and/or cleaning such as institutional cleaning and to compositions for use at low temperature.

### BACKGROUND OF THE INVENTION

Alpha-amylases (alpha-1,4-glucan-4-glucanohydrolases, E.C. 3.2.1.1) constitute a group of enzymes, which catalyses hydrolysis of starch and other linear and branched 1,4-gluosidic oligo- and polysaccharides.

There is a long history of industrial use of alpha-amylases in several known applications such as detergent, baking, brewing, starch liquefaction and saccharification e.g. in preparation of high fructose syrups or as part of ethanol production from starch. These and other applications of alpha-amylases are known and utilize alpha-amylases derived from microorganisms, in particular bacterial alpha-amylases.

Among the first bacterial alpha-amylases to be used were an alpha-amylase from B. licheniformis, also known as Termamyl which have been extensively characterized and the crystal structure has been determined for this enzyme. Alkaline amylases, such as AA560, form a particular group of alpha-amylases that have found use in detergents. Many of these known bacterial amylases have been modified in order to improve their functionality in a particular application.

Methods of increasing the thermostability of alpha-amylases have been well studied. Suzuki et al. (1989) disclose chimeric alpha-amylases, in which specified regions of a B. amyloliquefaciens alpha-amylase have been substituted for the corresponding regions of a B. licheniformis alpha-amylase. The chimeric alpha-amylases were constructed with the purpose of identifying regions responsible for thermostability. Such regions were found to include amino acid residues 177-186 and amino acid residues 255-270 of the B. amyloliquefaciens alpha-amylase. Igarashi et al. 1998 show that the thermostability of AmyS-type amylases can be increased by the deletion of two amino acid residues, R179−G180, (AmyS numbering) from a loop (F 178 to A184). However, Shiau et al. (2003) showed that an AmyS enzyme with deletion in the same loop has a lower specific activity for corn starch hydrolysis at high-temperature than the parent enzyme, negating one of the principal advantages of AmyS amylases.

For environmental reasons it has been increasingly important to lower the temperature in washing, dishwashing and/or cleaning processes. However, most enzymes including amylases have a temperature optimum which is above the temperature usually used in low temperature washing. Alpha-amylase is a key enzyme for use in detergent compositions and its use has become increasingly important for removal of starchy stains during laundry washing or dishwashing. Therefore, it is important to find alpha-amylase variants, which retain their wash performance, stain removal effect and/or activity when the temperature is lowered. However, despite the efficiency of current detergents enzyme compositions, there are many stains that are difficult to completely remove. These problems are compounded by the increased use of low (e.g., cold water) wash temperatures and shorter washing cycles. Thus, it is desirable to have amylolytic enzymes that can function under low temperature and at the same time preserve or increase other desirable properties such as specific activity (amylolytic activity), stability and/or wash performance.

Thus it is an object of the present invention to provide alpha-amylases variants which could be used in washing, dishwashing and/or cleaning processes at low temperature.

### SUMMARY OF THE INVENTION

The present invention concerns the use in a starch removing processes, such as laundry, dish wash, industrial or institutional cleaning of a variant or a variant of a parent alpha-amylase, comprising a deletion at one or more positions corresponding to positions 180, 181, 182, 183, 184 of the mature polypeptide of SEQ ID NO 3, wherein said variant having at least one improved property compared to an alpha-amylase having the identical amino acid sequence of said variant but not having the deletion in one or more of said position, or to the parent alpha-amylase or compared to an alpha-amylase having the amino acid sequence shown in SEQ ID NO 4, and wherein the temperature in the starch removing process is below 40 deg C., such as below 35 deg C.

The invention also concerns the use in washing or cleaning processes, such as laundry, dish wash, industrial or institutional cleaning of a variant or a variant of a parent alpha-amylase, comprising a deletion at one or more positions corresponding to positions 180, 181, 182, 183, 184 of the mature polypeptide of SEQ ID NO 3, wherein said variant having at least one improved property compared to an alpha-amylase having the identical amino acid sequence of said variant but not having the deletion in one or more of said position, or compared to the parent alpha-amylase or compared to an alpha-amylase having the amino acid sequence shown in SEQ ID NO 4 and wherein the temperature in laundry, dish wash, industrial and institutional cleaning is below 40 deg C., such as below 35 deg C.

The invention further concerns a composition comprising a surfactant and a variant or a variant of a parent alpha-amylase wherein the variant comprises a deletion at one or more positions corresponding to positions 180, 181, 182, 183, 184 of the mature polypeptide of SEQ ID NO: 3, wherein the variant has at least one improved property relative to an alpha-amylase having the identical amino acid sequence of said variant but not having the deletion in one or more of said position, or relative to the parent alpha-amylase or relative to an alpha-amylase having the amino acid sequence shown in SEQ ID NO 4 selected from the group consisting of improved activity, improved wash performance and improved stability.

The invention further concerns a method of cleaning comprising adding to a cleaning process a composition comprising at least one surfactant and a variant alpha-amylase, wherein the variant comprise a deletion at one or more positions corresponding to positions selected from the group consisting of 180, 181, 182, 183 and 184 (using the mature polypeptide of SEQ ID NO 3 for numbering), and wherein said variant comprises an amino acid sequence having at least 60% identity to amino acid sequence from the group consisting of SEQ ID NO 1, SEQ ID NO 2, SEQ ID NO 3, SEQ ID NO 4, SEQ ID NO 5 and SEQ ID NO 6, SEQ ID NO 7, SEQ ID NO 8, SEQ ID NO 9, SEQ ID NO 10, and SEQ ID NO 11, and wherein the variant has at least one improved property relative to an alpha-amylase having the identical amino acid sequence of said variant but not having the deletion at one or more of said positions or relative to an alpha-amylase having the amino acid sequence shown in SEQ ID NO 4 and wherein said cleaning process is performed at a temperature below 40 deg C., such as below 35 deg C.

The invention also provides an article of manufacture comprising (a) a container holding a cleaning composition comprising a surfactant and an alpha-amylase variant, wherein said variant comprises a deletion at one or more positions corresponding to positions selected from the group consisting of 180, 181, 182, 183 and 184 of the mature polypeptide of SEQ ID NO 3, (b) published material associated with the container informing that the cleaning composition is useful in a cleaning process which is performed at a temperature below 40, such as below 35 deg. C.

### DETAILED DESCRIPTION OF THE INVENTION

### Definitions

Alpha-Amylases (alpha-1,4-glucan-4-glucanohydrolases, E.C. 3.2.1.1) constitute a group of enzymes, which catalyze hydrolysis of starch and other linear and branched 1,4-glucosidic oligo- and polysaccharides.

Variant: The term “variant” is defined herein as an alpha-amylase comprising one or more, or one or several, alterations, such as substitutions, insertions, deletions, and/or truncations of one or more, or one or several, specific amino acid residues at one or more, or one or several, specific positions in the polypeptide.

Wild-Type Enzyme: The term “wild-type” alpha-amylase denotes an alpha-amylase expressed by a naturally occurring microorganism, such as an yeast or filamentous fungus found in nature.

Parent Enzyme: The term “parent” alpha-amylase as used herein means an alpha-amylase to which modifications, e.g., substitution(s), insertion(s), deletion(s), and/or truncation(s), are made to produce the enzyme variants of the present invention. Thus the parent or the original alpha-amylase is the alpha-amylase variant according to the invention without the specific mutations described in the present application. For example, if the variant is SP707+H183*+G184* (read as SP707 with the deletions H183*+G184*) then the parent is SP707 i.e. SP707 (SEQ ID NO 4) without the deletion of the amino acid at position 183 and 184. This term also refers to the polypeptide with which a variant is compared and aligned. The parent may be a naturally occurring (wild type) polypeptide, or it may be a variant thereof, prepared by any suitable means. For instance, the parent protein may be a variant of a naturally occurring polypeptide which has been modified or altered in the amino acid sequence. A parent may also be an allelic variant which is a polypeptide encoded by any of two or more alternative forms of a gene occupying the same chromosomal locus. In some cases the terms parent enzyme, original enzyme and wild type enzyme can be used interchangeably.

Starch removing process: The expression “starch removing process” relates to any kind of process whereby starch is removed (or converted) such as in washing processes where starch is removed from textile e.g. textile cleaning such as laundry. A starch removing process could also be hard surface cleaning such as dish wash or it could be cleaning processes in general such as industrial or institutional cleaning. The expression also comprises other starch removing processes or starch conversion, ethanol production, starch liquefaction, textile desizing, paper and pulp production, beer making and detergents in general.

Improved property: The term “improved property” is defined herein as a characteristic associated with a variant that is improved compared to the parent alpha-amylase, compared to an alpha-amylase having the identical amino acid sequence of the variant but not having the deletion at one or more of said specific positions or compared to an alpha-amylase having the amino acid sequence shown in SEQ ID NO 4. Such improved properties include, but are not limited to, increased amylolytic activity, e.g. when measured in Megazyme assay as described in the Methods section herein, increased wash performance, e.g. when measured in AMSA or in the beaker wash performance test as described in the Methods section herein, such as soil performance, e.g. performance to starch containing soils, stain removal, anti-greying, stability e.g. thermostability, storage stability, pH stability, or stability in the presence of builders, incl. chelants, stability in powder, liquid or gel detergent formulations or dishwashing compositions, altered temperature-dependent performance and activity profile, pH activity, substrate specificity, product specificity, and chemical stability. In an embodiment, improved properties include a combination of improved stability; improved wash and/or dish wash performance and/or improved activity in detergent, wherein improved stability includes both stability during storage in a concentrated detergent product and stability in the diluted detergent during wash. The improved property includes improved wash or dish wash performance at low temperature.

Activity: The terms “activity” and “specific activity” are used interchangeably in the present context and relate to the amyolytic activity measured by the amount of conversion of starch. The activity can be measured in e.g. a Megazyme assay as described in the Methods section below. In the present application the term “activity” is used interchangeably with “amyolytic activity”.

Improved activity: The term “improved activity” is defined herein as an altered activity of a variant enzyme relative (or compared) to the activity of the parent amylase or relative to an alpha-amylase having the identical amino acid sequence of said variant but not having the deletion at one or more of the specified positions or to the activity of an alpha-amylase having the amino acid sequence shown in SEQ ID NO 4, e.g. by increased starch conversion. The term “activity” includes amyolytic activity.

Wash performance: In the present context the term “wash performance” is used as an enzyme's ability to remove starch or starch-containing stains present on the object to be cleaned during e.g. laundry or hard surface cleaning, such as dish wash. The wash performance may be quantified by calculating the so-called intensity value (Int) defined in the description of AMSA or in the beaker wash performance test in the Methods section below.

Improved wash performance: The term “improved wash performance” is defined herein as a variant enzyme displaying an alteration of the wash performance of an amylase variant relative to the wash performance of the parent amylase or relative to an alpha-amylase having the identical amino acid sequence of said variant but not having the deletion at one or more of the specified positions or relative to the activity of an alpha-amylase having the amino acid sequence shown in SEQ ID NO 4, e.g. by increased stain removal. The term “wash performance” includes cleaning in general e.g. hard surface cleaning as in dish wash, but also wash performance on textiles such as laundry, and also industrial and institutional cleaning.

Stability: The term “stability” includes storage stability and reflects the stability of the amylase during time e.g. how much activity is retained when the amylase is kept in solution, in particular in detergent solution. For example, the alpha-amylase variant may have a residual activity, i.e. how much activity is retained, above 40% after 2 weeks at 37□deg C, wherein the activity is determined by the method described in the Methods section below. The stability is influenced on many factors e.g. pH, temperature, detergent composition e.g. amount of builder, surfactants etc. The amylase stability is measured using the method as described in the Methods section below.

Improved stability: The term “improved stability” is defined herein as an increased stability of a variant enzyme which is higher than the stability of the parent alpha-amylase or higher than the stability of an alpha-amylase having the identical amino acid sequence of said variant but not having the deletion at one or more of the specified positions, or higher than the stability of an alpha-amylase having the amino acid sequence shown in SEQ ID NO 4 The stability or the residual activity is determined by the method described in the Methods section below.

Low temperature: “Low temperature” is defined herein as a temperature at or below 40 deg C., such as below 38 deg C., such as below 37 deg C., such as below 35 deg C., such as below 34 deg C., such as below 32 deg C., such as below 30 deg C., such as below 25 deg C., such as below 20 deg C., such as below 15 deg C., such as below 10 deg C. or such as below 5 deg C.

Parent alpha-amylase: The “parent alpha-amylase” may in principle be any alpha-amylase for which it is desired to prepare a variant having improved activity, wash performance and/or stability at low temperature. Known alpha-amylases are derived from a wide selection of organisms including Bacteria, such as from species of the genus Bacillus e.g. Bacillus licheniformis; from species of fungi, such as Aspergillus oryzae (TAKA-amylase) or Aspergillus niger; from plants such as barley and from mammals. The parent alpha-amylse may in principle be any such alpha-amylase irrespective of the origin.

It is well known that a number of alpha-amylases produced by Bacillus spp. are highly identical on the amino acid level. Because of the substantial identity found between these alpha-amylases, they are considered to belong to the same class of alpha-amylases, namely the class of “Termamyl-like alpha-amylases”. Accordingly, in the present context, the term “Termamyl-like” alpha-amylase” is intended to indicate an alpha-amylase, in particular Bacillus alpha-amylase, which, at the amino acid level, exhibits a substantial identity i.e. at least 60% to the B. licheniformis alpha-amylase having the amino acid sequence shown in SEQ ID NO: 10 (Termamyl™), herein.

### Termamyl-Like Alpha-Amylases

The identity of a number of known Bacillus alpha-amylases can be found in the below Table 1:

TABLE 1

| | SEQ | Percent | | | | | | | |
|---|---|---|---|---|---|---|---|---|---|
| | ID | identity | | | | | | | |
| | NO: | 707 | AP1378 | BAN | BSG | SP690 | SP722 | AA560 | Termamyl |
| 707 | 4 | 100.0 | 86.4 | 66.9 | 66.5 | 87.6 | 86.2 | 95.5 | 68.1 |
| AP1378 | 9 | 86.4 | 100.0 | 67.1 | 68.1 | 95.1 | 86.6 | 86.0 | 69.4 |
| BAN | 7 | 66.9 | 67.1 | 100.0 | 65.6 | 67.1 | 68.8 | 66.9 | 80.7 |
| BSG | 8 | 66.5 | 68.1 | 65.6 | 100.0 | 67.9 | 67.1 | 66.3 | 65.4 |
| SP690 | 6 | 87.6 | 95.1 | 67.1 | 67.9 | 100.0 | 87.2 | 87.0 | 69.2 |
| SP722 | 3 | 86.2 | 86.6 | 68.8 | 67.1 | 87.2 | 100.0 | 86.8 | 70.8 |
| AA560 | 5 | 95.5 | 86.0 | 66.9 | 66.3 | 87.0 | 86.8 | 100.0 | 68.3 |
| Termamyl | 10 | 68.1 | 69.4 | 80.7 | 65.4 | 69.2 | 70.8 | 68.3 | 100.0 |

For instance, the B. licheniformis alpha-amylase comprising the amino acid sequence shown in SEQ ID NO: 10 (commercially available as Termamyl™) has been found to share about 81% identity with the B. amyloliquefaciens alpha-amylase comprising the amino acid sequence shown in SEQ ID NO: 7 (BAN) and about 65% identical with the B. stearothermophilus alpha-amylase comprising the amino acid sequence shown in SEQ ID NO: 8 (BSG). Further substantially identically alpha-amylases include SP722 and SP690 shown in SEQ ID NO: 3 and SEQ ID NO: 6, respectively, herein. Other amylases are the AA560 alpha-amylase derived from Bacillus sp. and shown in SEQ ID NO: 5, and the #707 (SP707) alpha-amylase derived from Bacillus sp., shown in SEQ ID NO: 4 and described by Tsukamoto et al. 1988. Further substantially identical protease is the KSM AP1378 alpha-amylase is disclosed in WO 97/00324 (from KAO Corporation) SEQ ID NO 9 or the SP.7-7 protease (from Henkel) SEQ ID NO 11. Another suitable parent amylase is the K 38 SEQ ID NO 1 or the B. circulans amylase with SEQ ID NO 2. Other suitable amylases are shown in SEQ ID NO 12 and 13.

Still further homologous alpha-amylases include the alpha-amylase produced by the B. licheniformis strain described in EP 0252666 (ATCC 27811), and the alpha-amylases identified in WO 91/00353 and WO 94/18314. Other commercial Termamyl-like alpha-amylases are comprised in the products sold under the following tradenames: Optitherm™ and Takatherm™ (Solvay); Maxamyl™ (available from Gist-brocades/Genencor), Spezym AA™ and Spezyme Delta AA™ (available from Genencor), and Keistase™ (available from Daiwa), Dex lo, GC 521 (available from Genencor) and Ultraphlow (from Enzyme Biosystems), Purastar™ ST 5000E, PURASTRA™ HPAM L, POWERASE™ (from Danisco), Spezyme FRED, GC358, ClearFlow AA.

The non-Termamyl-like alpha-amylase may, e.g., be a fungal alpha-amylase, a mammalian or a plant alpha-amylase or a bacterial alpha-amylase (different from a Termamyl-like alpha-amylase). Specific examples of such alpha-amylases include the Aspergillus oryzae TAKA alpha-amylase, the A. niger acid alpha-amylase, the Bacillus subtilis alpha-amylase, the porcine pancreatic alpha-amylase and a barley alpha-amylase. All of these alpha-amylases have elucidated structures, which are markedly different from the structure of a typical Termamyl-like alpha-amylase as referred to herein.

The fungal alpha-amylases mentioned above, i.e., derived from A. niger and A. oryzae, are highly homologous on the amino acid level and generally considered to belong to the same family of alpha-amylases. The fungal alpha-amylase derived from Aspergillus oryzae is commercially available under the trade name Fungamyl™.

The parent alpha-amylase may be a hybrid alpha-amylase, i.e., an alpha-amylase, which comprises a combination of partial amino acid sequences derived from at least two alpha-amylases.

The parent hybrid alpha-amylase may be one, which on the basis of amino acid homology and/or immunological cross-reactivity and/or DNA hybridization (as defined above) can be determined to belong to the Termamyl-like alpha-amylase family. In this case, the hybrid alpha-amylase is typically composed of at least one part of a Termamyl-like alpha-amylase and part(s) of one or more other alpha-amylases selected from Termamyl-like alpha-amylases or non-Termamyl-like alpha-amylases of microbial (bacterial or fungal) and/or mammalian origin.

Thus, the parent hybrid alpha-amylase may comprise a combination of partial amino acid sequences derived from at least two Termamyl-like alpha-amylases, or from at least one Termamyl-like and at least one non-Termamyl-like bacterial alpha-amylase, or from at least one Termamyl-like and at least one fungal alpha-amylase. The Termamyl-like alpha-amylase from which a partial amino acid sequence derives may be any of those specific any of those, such as the Termamyl-like, alpha-amylases referred to herein.

In one embodiment the parent Termamyl-like alpha-amylase is a hybrid Termamyl-like alpha-amylase identical to the Bacillus licheniformis alpha-amylase shown in SEQ ID NO: 10, except that the N-terminal 35 amino acid residues (of the mature protein) is replaced with the N-terminal 33 amino acid residues of the mature protein of the Bacillus amyloliquefaciens alpha-amylase shown SEQ ID NO: 7 (BAN) said hybrid may further have the following mutations: H156Y+A181T+N190F+A209V+Q264S (using the numbering in SEQ ID NO: 3) referred to as LE174. In another embodiment LE174 further comprising the mutations G48A, T49I, G107A, I201F, referred to as LE399.

In one embodiment the parent is SEQ ID NO: 8 with the mutations 1181*+G182*+N19F, referred to as TVB146.

In a preferred aspect of the invention the parent alpha-amylase is an alpha-amylase, which has the amino acid sequence shown in SEQ ID NO: 1, 2, 3, 5, 6, 7, 8, 9, 10 or 11 herein, or the amino acid sequence shown in SEQ ID NO: 4 which is also described in Tsukamoto et al., 1988. In another preferred aspect, the parent alpha-amylase is an alpha-amylase, which displays 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%, more preferred at least 99% of the mature polypeptide of SEQ ID NO: 1, 2, 3, 4, 5, 6, 7, 8, 9, 10 or 11.

In one aspect, the homologous polypeptides have an amino acid sequence that differs (e.g., deletion, insertion or substitution) by one or several amino acids, preferably ten amino acids, more preferably by nine, eight, seven, six, five amino acids, more preferably by four amino acids, even more preferably by three amino acids, most preferably by two amino acids, and even most preferably by one amino acid from the mature polypeptide of SEQ ID NO: 1, 2, 3, 4, 5, 6, 7, 8, 9, 10 or 11.

The parent alpha-amylase may be an alpha-amylase which displays immunological cross-reactivity with an antibody raised against an alpha-amylase having one of the amino acid sequences selected from the group consisting of SEQ ID NO 1, 2, 3, 4, 5, 6, 7, 8, 9, 10 and 11. In a preferred embodiment, the parent alpha-amylase is one wherein the antibody raised against the parent alpha-amylase displays an affinity or avidity for an alpha-amylase having one of the amino acid sequences shown in SEQ ID NO 1, 2, 3, 4, 5, 6, 7, 8, 9, 10 or 11 in a competitive assay technique such as e.g. ELISA or BiaCore, respectively, or that displays an affinity or avidity that is comparable to that of the parent alpha-amylase, and wherein the antibody raised against the alpha-amylase having one of the amino acid sequences shown in SEQ ID NO 1, 2, 3, 4, 5, 6, 7, 8, 9, 10 or 11 displays in said competitive assay technique an affinity or avidity for the parent alpha-amylase that is comparable with the affinity or avidity for the alpha-amylase having one of the amino acid sequences shown in SEQ ID NO 1, 2, 3, 4, 5, 6, 7, 8, 9, 10 or 11. In further embodiments, the parent alpha-amylase is one which has an affinity or avidity which is at least 70%, preferred at least 75% preferred at least 80%, preferred at least 85%, preferred at least 90%, preferred at least 95%, preferred at least 100%, preferred at least 110%, preferred at least 120%, especially preferred at least 125% of the affinity or avidity of the alpha-amylase having one of the amino acid sequences shown in SEQ ID NO 1, 2, 3, 4, 5, 6, 7, 8, 9, 10 or 11.

The parent alpha-amylase may also be an alpha-amylase which is encoded by a DNA sequence which hybridizes to the DNA sequence encoding the above-specified alpha-amylases.

Furthermore, when a particular variant of a parent alpha-amylase is referred to—in a conventional manner—by reference to modification (e.g., deletion or substitution) of specific amino acid residues in the amino acid sequence of a specific alpha-amylase, it is to be understood that variants of another alpha-amylase modified in the equivalent position(s) (as determined from the best possible amino acid sequence alignment between the respective amino acid sequences) are encompassed thereby.

In a particular aspect of the invention the parent alpha-amylase is a variant of a naturally occurring (wild type), prepared by any suitable means. For instance, the parent alpha-amylase may be a variant of a naturally occurring alpha-amylase which has been modified or altered in the amino acid sequence.

### Conventions for Designation of Variants

In the present invention, a specific numbering of amino acid residue positions in the alpha-amylase variants is employed. For example, by aligning the amino acid sequences of known alpha-amylases, it is possible to designate an amino acid position number to any amino acid residue in any alpha-amylase enzyme.

Using the numbering system originating from the amino acid sequence of the alpha-amylase disclosed in SEQ ID NO 3, aligned with the amino acid sequence of a number of other alpha-amylases, it is possible to indicate the position of an amino acid residue in an alpha-amylase in regions of structural homology.

In describing the various alpha-amylase variants of the present invention, the nomenclature described below is adapted for ease of reference. In all cases, the accepted IUPAC single letter or triple letter amino acid abbreviation is employed.

Substitutions: For an amino acid substitution, the following nomenclature is used: Original amino acid, position, substituted amino acid. Accordingly, the substitution of threonine with alanine at position 226 is designated as “Thr226Ala” or “T226A”. Multiple mutations are separated by addition marks (“+”), e.g., “Gly205Arg+Ser411Phe” or “G205R+S411F”, representing mutations at positions 205 and 411 substituting glycine (G) with arginine (R), and serine (S) with phenylalanine (F), respectively.

Deletions: For an amino acid deletion, the following nomenclature is used: Original amino acid, position*. Accordingly, the deletion of glycine at position 195 is designated as “Gly195*” or “G195*”. Multiple deletions are separated by addition marks (“+”), e.g., “Gly195*+Ser411*” or “G195*+S411*”. However, the nomenclature “195*” may be used when only the position of the deleted amino acid residue is specified.

Insertions: For an amino acid insertion, the following nomenclature is used: Original amino acid, position, original amino acid, new inserted amino acid. Accordingly the insertion of lysine after glycine at position 195 is designated “Gly195GlyLys” or “G195GK”. Multiple insertions of amino acids are designated [Original amino acid, position, original amino acid, new inserted amino acid #1, new inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example the sequences would thus be:

| Parent: | Variant: |
|---|---|
| 195 | 195 195a 195b |
| G | G - K - A |

Degenerate indications: For degenerate indications where an amino acid residue identical to the existing amino acid residue is inserted, degeneracy in the nomenclature arises. For example, a glycine inserted after the glycine in the above example would be indicated by “G195GG”. Given that an alanine were present at position 194, the same actual change could just as well be indicated as “A194AG”:

| | Parent: | Variant: |
|---|---|---|
| Numbering I: | 194 195 | 194 195 195a |
| Sequence: | A - G | A - G - G |
| Numbering II: | | 194 194a 195 |

Such instances will be apparent to the skilled person, and the indication “G195GG” and corresponding indications for this type of insertion is thus meant to comprise such equivalent degenerate indications.

If amino acid sequence segments are repeated in the parent polypeptide and/or in the variant, equivalent degenerate indications arise, also when alterations other than insertions are listed such as deletions and/or substitutions. For example, the deletion of two consecutive amino acids “AG” in the sequence “AGAG” from position 194-97 may be written as “A194*+G195*” or “A196*+G197*”:

| | Parent: | Variant: |
|---|---|---|
| Numbering I: | 194 195 196 197 | 194 195 |
| Sequence: | A - G - A - G | A - G |
| Numbering II: | | 196 197 |

Multiple modifications: Variants comprising multiple modifications are separated by addition marks (“+”), e.g., “Arg170Tyr+Gly195Glu” or “R170Y+G195E” representing modifications at positions 170 and 195 substituting tyrosine and glutamic acid for arginine and glycine, respectively. Thus, “Tyr167Gly,Ala,Ser,Thr+Arg170Gly,Ala,Ser,Thr” designates the following variants:

| | | |
|---|---|---|
| “Tyr167Gly + Arg170Gly”, | “Tyr167Gly + Arg170Ala”, | “Tyr167Gly + Arg170Ser”, |
| “Tyr167Gly + Arg170Thr”, | “Tyr167Ala + Arg170Gly”, | “Tyr167Ala + Arg170Ala”, |
| “Tyr167Ala + Arg170Ser”, | “Tyr167Ala + Arg170Thr”, | “Tyr167Ser + Arg170Gly”, |
| “Tyr167Ser + Arg170Ala”, | “Tyr167Ser + Arg170Ser”, | “Tyr167Ser + Arg170Thr”, |
| “Tyr167Thr + Arg170Gly”, | “Tyr167Thr + Arg170Ala”, | “Tyr167Thr + Arg170Ser”, |
| and “Tyr167Thr + Arg170Thr”. | | |

This nomenclature is particularly relevant to modifications involving substituting, inserting or deleting amino acid residues having specific common properties. Such modifications are referred to as conservative amino acid modification(s). Examples of conservative modifications are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid modifications, which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as the reverse (Taylor, 1986).

### Characteristics of Amino Acid Residues

It is well known in the art that a so-called conservative substitution of one amino acid residue to a similar amino acid residue is expected to produce only minor change in the characteristic of the enzyme. Thus, the amino acid residues can be divided in groups of conservative amino acid substitutions:

### Charged Amino Acids:

### Asp, Glu, Arg, Lys, His

Negatively Charged Amino Acids (with the Most Negative Residue First):

### Asp, Glu

Positively Charged Amino Acids (with the Most Positive Residue First):

### Arg, Lys, H is

### Neutral Amino Acids:

### Gly, Ala, Val, Leu, Ile, Phe, Tyr, Trp, Met, Cys, Asn, Gln, Ser, Thr, Pro

Hydrophobic Amino Acid Residues (with the Most Hydrophobic Residue Listed Last):

### Gly, Ala, Val, Pro, Met, Leu, Ile, Tyr, Phe, Trp,

Hydrophilic Amino Acids (with the Most Hydrophilic Residue Listed Last):

### Thr, Ser, Cys, Gln, Asn

### Variants and Use

The inventors have surprisingly found that certain variants of alpha-amylases have much improved activity relative to the parent alpha-amylase or relative to an alpha-amylase having the identical amino acid sequence of said variant but not having mutations at one or more specified positions, and at the same time showed significantly improved wash performance at low temperature. This result was not expected because lowering the temperature both affects the performance of the enzymes, but it also makes the various components in the stains be removed more inaccessible, probably due to a slower hydration. In addition, the inventors found that the stability of the variants relative to the parent or relative to an alpha-amylase having the identical amino acid sequence of said variant but not having mutations at one or more specified positions was much improved.

Accordingly, in a first aspect the present invention relates to the use in a starch removing process of a variant or a variant of a parent alpha-amylase, wherein the variant comprises a deletion at one or more, or one or several, positions corresponding to positions selected from the group consisting of 180, 181, 182, 183, and 184 of the mature polypeptide of SEQ ID NO 3, wherein said variant having at least one improved property compared to an alpha-amylase having the identical amino acid sequence of said variant but not having the deletion at one or more, or one or several, of said positions or compared to the parent alpha-amylase or compared to the alpha-amylase with SEQ ID NO 4, and wherein the temperature in the starch removing process is below 40 deg C., such as below 35 deg C. In particular a single or double deletion variant and a number of other single mutant variants have improved wash performance and activity at low temperature.

In the present context, the expression “one or more” or “, or “one or several” means that the variant of the alpha-amylase has 1, 2, 3, 4 or 5 deletions at positions corresponding to positions selected from the group consisting of 180, 181, 182, 183, and 184 of the mature polypeptide of SEQ ID NO 3.

Accordingly, in a second aspect the present invention relates to the use in laundry, dish wash, industrial or institutional cleaning of a variant or a variant of a parent alpha-amylase, comprising a deletion at one or more, or one or several, positions corresponding positions selected from the group consisting of 180, 181, 182, 183 and 184 of the mature polypeptide of SEQ ID NO 3, wherein said variant having at least one improved property compared to an alpha-amylase having the identical amino acid sequence of said variant but not having the deletion at one or more, or one or several, of said positions or compared to the parent alpha-amylase or compared to the alpha-amylase with SEQ ID NO 4 and wherein the temperature in laundry, dish wash, industrial and institutional cleaning is below 40 deg, such as below 35 deg C.

In a preferred embodiment the alpha-amylase is a variant of the parent alpha-amylase disclosed in SEQ ID NO 4 herein having a deletion in positions F180, R181, G182, H183 and/or G184, preferably wherein said alpha-amylase variant further has a substitution in or corresponding to position N195F, especially wherein the parent alpha-amylase (SP707) has one or more, or one or several, of the following deletions/substitutions in SEQ ID NO: 4 herein: Delta (R181+G182); Delta (R181+H183); Delta (G182+H183); Delta (R181+G184), Delta (G182+G184), Delta (H183+G184); Delta (H183+G184)+N195F; R181Q+N445Q+K446N; Delta (H183−G184)+R181Q. In another preferred embodiment the parent alpha-amylase (SP707) has one or more of the following deletions/substitutions in SEQ ID NO: 4 herein: Delta (R181+G182); Delta (G182+H183); Delta (R181,G184), Delta (G182+G184), Delta (H183+G184); Delta (H183+G184)+N195F; Delta (H183+G184)+R181Q, N445Q, K446N; Delta (H183+G184)+R181Q, Delta (H183+G184) and one or more of the following substitutions: R118K, N195F, R320K, R458K. It will be clear to the person skilled in the art that Delta (R181+G182) means deletion at positions 181 and 182.

In a preferred embodiment of the invention the alpha-amylase is a variant of the parent alpha-amylase disclosed in SEQ ID NO 4 herein having a deletion in positions H183 and/or G184. In a particular preferred embodiment the alpha-amylase variant is SP707+H183*+G184*. In another embodiment the alpha-amylase is a variant of the parent alpha-amylase disclosed in SEQ ID NO 5 herein having a deletion in positions D183 and/or G184, preferably wherein said alpha-amylase variant further has a substitution in or corresponding to position N195F, especially wherein the variant alpha-amylase has one or more of the following deletions/substitutions in SEQ ID NO 5 herein: Delta (R181+G182); Delta (G182+D183); Delta (D183−G184).

In another preferred embodiment the variant alpha-amylase has one or more of the following deletions/substitutions in SEQ ID NO: 5 herein: Delta (R181+G182); Delta (G182+D183); Delta (D183+G184); Delta (R181+G184), Delta (G182+G184), Delta (D183+G184)+N195F; Delta (D183+G184)+R118K, N195F; Delta (D183+G184)+R118K, N195F, R320K; Delta (D183+G184)+N195F, R118K, R320K, R458K or this variant with the following mutations M9L, G149A, G182T, G186A, M202L, T257I, Y295F, N299Y, M323T, A339S, E345R.

In another preferred embodiment the alpha-amylase is a variant of SP722 (SEQ ID NO 3), SP707 (SEQ ID NO 4), AA560 (SEQ ID NO 5) or SP690 (SEQ ID NO 6), such as a variant comprising Delta (R181−G182); Delta (G181−D183), Delta (G181−H183), Delta (G181,G184), Delta (G182+G184), Delta (G181−H183); Delta (G182−D183), Delta (G182−H183), Delta (G182−T183); Delta (D183−G184), Delta (H183−G184) or Delta (T183−G184) (numbering according to SEQ ID NO 3) or an alpha-amylase variant wherein the variants further comprises one or more of the following substitutions: M9L, M202L, V214T, M323T, M382Y or M9L, M202L, V214T, M323T and E345R. In another preferred embodiment the alpha-amylase comprises Asn-Gly-Thr-Met-Met-Gln-Tyr-Phe-Glu-Trp in its N-terminal amino acid region.

In a preferred embodiment the variant alpha-amylase is a variant of SP707 (SEQ ID NO 4) including any of SP707+R181*; SP707+G182*; SP707+H183*; SP707+G184*; SP707+R181*+G182*, SP707+R181*+H183*; SP707+R181*+G184*; SP707+G182*+G184*; SP707+G182*+H183*, SP707+H183*+G184*; SP707+R181*+G182*+N195F; SP707+R181*+H183*+N195F; SP707+G182*+H183*+N195F; SP707+H183*+G184*+N195F; SP707+R181*+G182*+M202L; SP707+R181*+H183*+M202L; SP707+G182*+H183*+M202L; SP707+D183*+G184*+M202L; SP707+R181*+G182*+N195F+M202L; SP707+R181*+H183*+N195F+M202L SP707+G182*+H183*+N195F+M202L; SP707+H183*+G184*+N195F+M202L; SP707+R181*+G182*+R181Q; SP707+R181*+H183*+R181Q; SP707+G182*+H183*+R181Q; SP707+H183*+G184*+R181Q; SP707+R181*+G182*+R118K+N195F+R320K+S458K; SP707+R181*+H183*+R118K+N195F+R320K+S458K; SP707+G182*+H183*+R118K+N195F+R320K+5458K; SP707+H183*+G184*+R118K+N195F+R320K+S458K; SP707+R181*+G182*+H183G

In another preferred embodiment the variant alpha-amylase is a variant of SP722 (SEQ ID NO 3) including any of SP722+R181*; SP722+G182*; SP722+D183*; SP722+G184*; SP722+R181*+G182*, SP722+R181*+D183*; SP722+G182*+D183*, SP722+R181*+G184*; SP722+G182*+G184*; SP722+D183*+G184*; SP722+R181*+G182*+N195F; SP722+R181*+D183*+N195F; SP722+G182*+D183*+N195F; SP722+D183*+G184*+N195F; SP722+R181*+G182*+M202L; SP722+R181*+D183*+M202L; SP722+G182*+D183*+M202L; SP722+D183*+G184*+M202L; SP722+R181*+G182*+N195F+M202L; SP722+R181*+D183*+N195F+M202L; SP722+G182+D183*+N195F+M202L; SP722+D183*+G184*+N195F+M202L; SP722+R181*+G182*+R181Q; SP722+R181*+D183*+R181Q; SP722+G182*+D183*+R181Q; SP722+D183*+G184*+R181Q; SP722+R181*+G182*+L118K+N195F+H458K; SP722+R181*+D183*L118K+N195F+H458K; SP722+G182*+D183*+L118K+N195F+H458K; SP722+D183*+G184*+L118K+N195F+H458K; SP722+R181*+G182*+D183G.

In another preferred embodiment the variant alpha-amylase is a variant of AA560 (SEQ ID NO 5) including any of AA560+R181*; AA560+G182*; AA560+T183*; AA560+G184*; AA560+R181*+G182*, AA560+G182*+D183*, AA560+R181*+G184*; AA560+G182*+G184*; AA560+D183*+G184*; AA560+R181*+G182*+N195F; AA560+R181*+D183*+N195F; AA560+G182*+D183*+N195F; AA560+D183*+G184*+N195F; AA560+R181*+G182*+M202L;; AA560+R181*+D183*+M202L; AA560+G182*+D183*+M202L; AA560+D183*+G184*+M202L; AA560+R181*+G182*+N195F+M202L; AA560+R181*+D183*+N195F+M202L; AA560+G182*+D183*+N195F+M202L; AA560+D183*+G184*+N195F+M202L; AA560+R181*+G182*+R118K+N195F+R320K+T458K; AA560+R181*+D183*+R118K+N195F+R320K+T458K; AA560+G182*+D183*+R118K+N195F+R320K+T458K; AA560+D183*+G184*+R118K+N195F+R320K+T458K; AA560+R181*+G182*+D183G.

In another preferred embodiment the variant alpha-amylase is a variant of SP690 (SEQ ID NO 6) including any of SP690+R181*; SP690+G182*; SP690+T183*; SP690+G184*; SP690+R181*+G182*, SP690+R181*+T183*; SP690+G182*+T183*, SP690+T183*+G184*; SP690+R181*+G184*; SP690+G182*+G184*; SP690+R181*+G182*+N195F; SP690+R181*+T183*+N195F; SP690+G182*+T183*+N195F; SP690+T183*+G184*+N195F; SP690+R181*+G182*+M202L; SP690+R181*+T183*+M202L SP690+G182*+T183*+M202L; SP690+T183*+G184*+M202L; SP690+R181*+G182*+N195F+M202L; SP690+R181*+T183*+N195F+M202L; SP690+G182*+T183*+N195F+M202L; SP690+T183*+G184*+N195F+M202L; SP690+R181*+G182*+R118K+N195F+R320K+T458K; SP690+R181*+T183*+R118K+N195F+R320K+T458K; SP690+G182*+T183*+R118K+N195F+R320K+T458K; SP690+T183*+G184*+R118K+N195F+R320K+T458K; SP690+R181*+G182*+D183G

It will be understood, that “SP722+R181*+G182*+N195F” means the Bacillus spp. alpha-amylase has been mutated as follows: deletions in positions R181 and G182 and a substitution from Asn (N) to Phe (F) in position 195 wherein the numbering corresponds to SEQ ID NO 3 (counting as if the deleted positions are still present i.e. the numbering does not shift down by two when deleting two positions).

In one preferred aspect, the variant alpha-amylase comprises an amino acid sequence having a degree of identity to the mature polypeptide of SEQ ID NO 4 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In another preferred aspect, the variant alpha-amylase comprises an amino acid sequence having a degree of identity to the mature polypeptide of SEQ ID NO 3, SEQ ID NO 5 or SEQ ID NO 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In one aspect the variant alpha-amylase has an amino acid sequence that differs by one or several amino acids, preferably ten amino acids, preferably by nine amino acids, preferably by eight amino acids, preferably by seven amino acids, preferably by six amino acids, preferably by five amino acids, more preferably by four amino acids, even more preferably by three amino acids, or even most preferably by two or one amino acid from the mature polypeptide of SEQ ID NO 3, 4, 5 or 6.

A preferred aspect of the invention concerns the use in a starch removing process of a variant or a variant of a parent alpha-amylase, wherein the variant comprises a deletion at one or more positions corresponding to positions selected from the group comprising 180, 181, 182, 183 and 184 of the mature polypeptide of SEQ ID NO 3, wherein said variant having at least one improved property compared to an alpha-amylase having the identical amino acid sequence of said variant but not having the deletion at one or more of said positions or compared to the parent alpha-amylase or compared to the alpha-amylase with SEQ ID NO 4, and wherein the temperature in the starch removing process is below 40 deg C., such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

A preferred aspect of the invention concerns the use in laundry, dish wash, industrial or institutional cleaning of a variant or a variant of a parent alpha-amylase, wherein the variant comprises a deletion at one or more positions corresponding to positions selected from the group comprising 180, 181, 182, 183 and 184 of the mature polypeptide of SEQ ID NO 3, wherein said variant having at least one improved property compared to an alpha-amylase having the identical amino acid sequence of said variant but not having the deletion at one or more of said positions or compared to the parent alpha-amylase or compared to the alpha-amylase with SEQ ID NO 4 and wherein the temperature in laundry, dish wash, industrial and institutional cleaning is below 40 deg, such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

Another preferred aspect concerns the use, wherein laundry, dish wash and industrial or institutional cleaning is performed at temperature in the range of 5-40 deg C., such as in the range of 5-35 deg C.

Another preferred aspect concern the use, wherein the laundry, dish wash or industrial cleaning is performed at temperature ranges selected from 10-40 deg C., 10-35 deg C., 10-20 deg C., 15-40 deg C., 15-20 deg C., 5-25 deg C., 5-20 deg C., 5-15 deg C., 5-10 deg C.

In a particularly preferred aspect of the invention the variant has retained its wash performance or has improved wash performance compared to the parent alpha-amylase or compared to the alpha-amylase with SEQ ID NO 4 when measured in AMSA or in the beaker wash performance test as described in the Methods section herein.

In another preferred embodiment the variant comprises a combination of deletions at two or more positions corresponding to positions selected from the group comprising F180, R181, G182, D183, G184 of the mature polypeptide of SEQ ID NO 3 in particular the deletions F180*+R181*; R181*+D183*; R181*+G182*; G182*+D183*R181*+G184*, G182*+G184* or D183*+G184*. Such pairwise deletions may thus be the following:

### F180*+R181*; R181*+G182*; D183*+G184*; R181*+D183*; G182*+D183*; G182*+G184*; and R181*+G184* (SEQ ID NO. 3);

### F180*+R181*; R181*+G182*; H183*+G184*; R181*+H183*; G182*+H183*; G182*+G184*; and R181*+G184* (SEQ ID NO. 4);

### F180*+R181*; R181*+G182*; D183*+G184*; R181*+D183*; G182*+D183*; G182*+G184*; and R181*+G184* (SEQ ID NO. 5);

### F180*+R181*; R181*+G182*; T183*+G184*; R181*+T183*; G182*+T183*; G182*+G184*; and R181*+G184* (SEQ ID NO. 6);

or equivalents of these pairwise deletions in another alpha-amylase meeting the requirements of a parent alpha-amylase in the context of the present invention.

In the present context, the expression “two or more” means that the variant of the alpha-amylase has 2, 3, 4 or 5 deletions at positions corresponding to positions selected from the group consisting of 180, 181, 182, 183, and 184 of the mature polypeptide of SEQ ID NO 3.

In a particularly preferred embodiment the variant according to the present invention comprises a combination of deletions of two or more positions corresponding to positions selected from the group comprising 180, 181, 182, 183 and 184 of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

A preferred aspect of the invention concerns the use in laundry, dish wash, industrial or institutional cleaning of a variant or a variant of a parent alpha-amylase, comprising a combination of deletions of two or more positions corresponding to positions selected from the group comprising 180, 181, 182, 183 and 184 of the mature polypeptide of SEQ ID NO 3, wherein said variant having at least one improved property compared to an alpha-amylase having the identical amino acid sequence of said variant but not having the deletion at two or more of said positions or compared to the parent alpha-amylase or compared to the alpha-amylase with SEQ ID NO 4, and wherein the temperature in laundry, dish wash, industrial and institutional cleaning is below 40 deg, such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

A preferred embodiment relates to a variant or a variant of a parent alpha-amylase, wherein the variant comprises a combination of deletions of two or more positions corresponding to positions 180*+181*; 181*+182*; 181*+183*; 182*+183*, 181*+184*, 182*+184*; or 183*+184* of the mature polypeptide of SEQ ID NO 3 and wherein the variant alpha-amylase having a degree of identity to the mature polypeptide of SEQ ID NO 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

Thus one aspect of the invention relates to the use in laundry, dish wash industrial or institutional cleaning of a variant or a variant of a parent alpha-amylase, wherein said variant comprises a combination of deletions of two or more positions corresponding to positions 180*+181*; 181*+182*; 181*+183*; 182*+183*, 181*+184*, 182*+184*, or 183*+184* of the mature polypeptide of SEQ ID NO 3, wherein said variant having at least one improved property compared to an alpha-amylase having the identical amino acid sequence of said variant but not having the deletion at two or more of said positions or compared to the parent alpha-amylase or compared to the alpha-amylase with SEQ ID NO 4 and wherein the temperature in laundry, dish wash, industrial and institutional cleaning is below 40 deg, such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

Another embodiment relates to a variant or a variant of a parent alpha-amylase, wherein the variant comprises a combination of deletions of two or more positions corresponding to positions selected for the group comprising 180, 181, 182, 183 and 184 of the mature polypeptide of SEQ ID NO 3 and further comprises a substitution at any, or one or more, of the position(s) corresponding to any of the positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

Thus one aspect of the invention relates the use in laundry, dish wash, industrial or institutional cleaning of a variant or a variant of a parent alpha-amylase, comprising a combination of deletions of two or more positions corresponding to positions selected from the group comprising 180, 181, 182, 183 and 184 of the mature polypeptide of SEQ ID NO 3 and further comprises a substitution at one or more of the position(s) corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3, wherein said variant having at least one improved property compared to an alpha-amylase having the identical amino acid sequence of said variant but not having the deletion at two or more of said positions nor the further substitutions or compared to the parent alpha-amylase or compared to the alpha-amylase with SEQ ID NO 4 and wherein the temperature in laundry, dish wash, industrial and institutional cleaning is below 40 deg C., such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C, such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

Yet another embodiment relates to a variant or a variant of a parent alpha-amylase, wherein the variant comprises a combination of deletions of two or more positions corresponding to positions 180*+181*, 181*+182*; 181*+183*; 182*+183*; 181*+184*, 182*+184*, or 183*+184* of the mature polypeptide of SEQ ID NO 3 and further comprises a substitution at any, or one or more, of the position(s) corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase having a degree of identity to the mature polypeptide of SEQ ID NO 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

Thus one aspect of the invention relates the use in laundry, dish wash, industrial or institutional cleaning of a variant or a variant of a parent alpha-amylase, comprising a combination of deletions of two or more positions corresponding to positions 180*+181*, 181*+182*; 181*+183*; 182*+183*; 181*+184*, 182*+184*, or 183*+184* of the mature polypeptide of SEQ ID NO 3 and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3, wherein said variant having at least one improved property compared to an alpha-amylase having the identical amino acid sequence of said variant but not having the deletion at two or more of said positions nor the further substitutions or compared to the parent alpha-amylase or compared to the alpha-amylase with SEQ ID NO 4 and wherein the temperature in laundry, dish wash, industrial and institutional cleaning is below 40 deg, such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C, such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

Yet another embodiment relates to a variant or a variant of a parent alpha-amylase, wherein the variant comprises a combination of deletions of two or more positions corresponding to positions selected from the group consisting of 180, 181, 182, 183 and 184 of the mature polypeptide of SEQ ID NO 3 and further comprising any, or one or more, of the following substitutions N28R, S36D, T40S, V75I, S83N, T90N, S91A, N94S, R118K, T132S, R142K, S154N, R172K, A186G, N195F, Q280N, N311Q, R320K, S323M, R383K, K400R, S437N, S452T, R458K and T459A (using numbering according to SEQ ID NO 3) and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In the case where the original or parent amino acid residue may be any amino acid residue, a short hand notation may at times be used indicating only the position and substituted amino acid e.g. 40S. Another way is to indicate the original or parent amino acid with an X. Such a notation is particular relevant in connection with modification(s) in homologous alpha-amylases similarly when the identity of the substituting amino acid residue(s) is immaterial.

The skilled person would know that using numbering according to SEQ ID NO 3 means using SEQ ID NO 3 for countering not that the parent necessarily is SEQ ID NO 3 but simply that the positions to be altered are defined according to SEQ ID NO 3. Therefore, another way of describing the specific substitutions is to indicate the amino acid to be altered with an X. Thus X40S means that any amino acid present at position 40 could be substituted with S reflecting that different alpha-amylase can be used as parent alpha-amylase.

Thus another embodiment relates to a variant or a variant of a parent alpha-amylase, wherein the variant comprises a combination of deletions of two or more positions corresponding to positions selected from the group consisting of 180, 181, 182, 183 and 184 of the mature polypeptide of SEQ ID NO 3 and further comprises any, or one or more, of the specific substitutions X28R, X36D, X405, X75I, X83N, X90N, X91A, X94S, X118K, X132S, X142K, X154N, X172K, X186G, X195F, X280N, X311Q, X320K, X323M, X383K, X400R, X437N, X452T, X458K and X459A (using numbering according to SEQ ID NO 3) and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

It is to be understood that when the parent alpha-amylase e.g. SEQ ID NO 3, 4, 5 or 6 already have the substituted amino acid no substitution is made e.g. if the parent already has S in position 40 no substitution is made at this position.

Thus one aspect of the invention relates to the use in laundry, dish wash, industrial or institutional cleaning of a variant or a variant of a parent alpha-amylase, comprising a combination of deletions of two or more positions corresponding to positions 180*+181*, 181*+182*; 181*+183*; 182*+183*; 181*+184*, 182*+184*, or 183*+184* of the mature polypeptide of SEQ ID NO 3 and further comprises any, or one or more, of the specific substitutions X28R, X36D, X405, X75I, X83N, X90N, X91A, X94S, X118K, X132S, X142K, X154N, X172K, X186G, X195F, X280N, X311Q, X320K, X323M, X383K, X400R, X437N, X452T, X458K and X459A (using numbering according to SEQ ID NO 3) and wherein the variant alpha-amylase having a degree of identity to the mature polypeptide of SEQ ID NO 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%, wherein said variant has at least one improved property compared to an alpha-amylase having the identical amino acid sequence of said variant but not having the deletion at two or more of said positions nor further substitutions or compared to the parent alpha-amylase or compared to an alpha-amylase having the amino acid sequence shown in SEQ ID NO 4, and wherein the temperature in laundry, dish wash, industrial and institutional cleaning is below 40 deg C., such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

In a particularly preferred embodiment the variant according to the present invention comprises a combination of deletions at two or more positions selected from the group consisting of F180, R181, G182, H183 and G184 (using SEQ ID NO 3 for numbering) of the mature polypeptide of SEQ ID NO: 4 in particular the deletions F180*+G182*; R181*+G182*; R181*+H183*; G182*+H183*, R181*+G184*, G182*+G184*, or H183*+G184*. In yet another aspect the variants of the invention comprises the deletions R181*+G182*; R181*+H183*; G182*+H183*; or H183*+G184* (using SEQ ID NO 3 for numbering) and further comprises one or more substitution(s) at any of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO: 4. In another aspect the variants of the invention comprises the deletions R181*+G182*; R181*+H183*; G182*+H183*; R181*+G184*, G182*+G184*, or H183*+G184* (using SEQ ID NO 3 for numbering) and further comprises any, or one or more, of the specific substitutions at any, or one or more, position(s) N28R, S36D, T40S, V75I, S83N, T90N, S91A, N94S, R118K, T132S, R142K, S154N, R172K, A186G, N195F, Q280N, N311Q, R320K, S323M, R383K, K400R, S437N, S452T, R458K and T459A of the mature polypeptide of SEQ ID NO: 4.

Thus one aspect of the invention relates the use in laundry, dish wash, industrial or institutional cleaning of a variant or a variant of a parent alpha-amylase, comprising a combination of deletions of two or more positions F180*+G182*; R181*+G182*; R181*+H183*; G182*+H183*, R181*+G184*, G182*+G184*, or H183*+G184 (using SEQ ID NO 3 for numbering) of the mature polypeptide of SEQ ID NO 4 and further comprises any, or one or more, of the specific substitutions at one or more position(s) N28R, S36D, T405, V75I, S83N, T90N, S91A, N94S, R118K, T132S, R142K, S154N, R172K, A186G, N195F, Q280N, N311Q, R320K, S323M, R383K, K400R, S437N, S452T, R458K and T459A of the mature polypeptide of SEQ ID NO: 4 or of an amino acid sequence having a degree of identity to the mature polypeptide of SEQ ID NO: 4 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% identity, more preferred at least 96%, more preferred at least 97%, more preferred at least 98%, wherein said variant having at least one improved property compared to an alpha-amylase having the identical amino acid sequence of said variant but not having the deletion at two or more of said positions nor having further substitutions, or compared to the parent alpha-amylase or to an alpha-amylase having the amino acid sequence shown in SEQ ID NO 4, and wherein the temperature in laundry, dish wash, industrial and institutional cleaning is below 40 deg, such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C and/or below, such as 20 deg C. and/or below, such as 15 deg C. and/or below, such as 10 deg C. and/or below, such as 5 deg C. and/or below, such as 2 deg C. and/or below or 1 deg C. and/or below.

Thus one aspect of the invention concerns the use of a variant, wherein the deletions are or are equivalent to R181*+G182*; R181*+H183*; G182*+H183*; R181*+G184*, G182*+G184*, or H183*+G184 of SEQ ID NO 4 and wherein the variant alpha-amylase comprises an amino acid sequence having a degree of identity to the mature polypeptide of SEQ ID NO: 4 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In a particular embodiment of the invention the variant comprises the following alterations:

### G182*+D183*+N28R

### G182*+D183*+S36D

### G182*+D183*+T40S

### G182*+D183*+V75I

### G182*+D183*+S83N

### G182*+D183*+T90N

### G182*+D183*+S91A

### G182*+D183*+N94S

### G182*+D183*+R118K

### G182*+D183*+T132S

### G182*+D183*+R142K

### G182*+D183*+S154N

### G182*+D183*+R172K

### G182*+D183*+A186G

### G182*+D183*+N195F

### G182*+D183*+Q280N

### G182*+D183*+N311Q

### G182*+D183*+R320K

### G182*+D183*+S323M

### G182*+D183*+R383K

### G182*+D183*+K400R

### G182*+D183*+S437N

### G182*+D183*+S452T

### G182*+D183*+R458K

### G182*+D183*+T459A

where each position corresponds to a position of the amino acid sequence shown in SEQ ID NO 3.

In a particularly preferred embodiment of the invention the variant comprises the following alterations in the alpha-amylase having SEQ ID NO 4:

### G182*+H183*+N28R

### G182*+H183*+S36D

### G182*+H183*+T40S

### G182*+H183*+V75I

### G182*+H183*+S83N

### G182*+H183*+T90N

### G182*+H183*+S91A

### G182*+H183*+N94S

### G182*+H183*+R118K

### G182*+H183*+T132S

### G182*+H183*+R142K

### G182*+H183*+S154N

### G182*+H183*+R172K

### G182*+H183*+A186G

### G182*+H183*+N195F

### G182*+H183*+Q280N

### G182*+H183*+N311Q

### G182*+H183*+R320K

### G182*+H183*+S323M

### G182*+H183*+R383K

### G182*+H183*+K400R

### G182*+H183*+5437N

### G182*+H183*+S452T

### G182*+H183*+R458K

### G182*+H183*+T459A

where each position corresponds to a position of the amino acid sequence shown in SEQ ID NO: 3.

In a preferred aspect the variants according to the invention have improved activity at low temperature compared to an alpha-amylase having the identical amino acid sequence of said variant but not having the deletion at two or more of said positions or compared to the parent or compared to an alpha-amylase having the amino acid sequence shown in SEQ ID NO 4. This is to be understood in the present context as variants having higher activity relative to an alpha-amylase having the identical amino acid sequence of said variant but not having the deletion at two or more of said positions or relative to the parent or to an alpha-amylase having the amino acid sequence shown in SEQ ID NO 4 when the activity of the variant is measured in the Megazyme assay as described in Example section.

One aspect of the invention relates to the use in a starch removing process of a variant or a variant of a parent alpha-amylase wherein the activity of the amylase variant at 20 deg C. and at pH 8 is at least 10%, such as at least 20%, preferably at least 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% increased relative to an alpha-amylase having the identical amino acid sequence of said variant but not having no deletion at said positions or relative to the parent alpha-amylase or to an alpha-amylase with SEQ ID NO 4 when measured in the Megazyme assay as described in the Examples.

Thus one aspect of the invention relates to the use in laundry, dish wash, industrial or institutional cleaning of a variant or a variant of a parent alpha-amylase wherein the activity of the amylase variant according to the invention at 20 deg C. and at pH 8 is at least 10%, such as at least 20%, preferably at least 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% increased relative to an alpha-amylase having the identical amino acid sequence of said variant but not having deletion or relative to the parent alpha-amylase or to an alpha-amylase with SEQ ID NO 4 when measured in the Megazyme assay as described in the Examples.

Another aspect of the invention concerns the use in laundry, dish wash, industrial or institutional cleaning of a variant or a variant of a parent alpha-amylase wherein the increase in activity of the amylase variant at 20 deg C. and at pH 10 is at least 10%, such as at least 20%, preferably at least 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% increased relative to an alpha-amylase having the identical amino acid sequence of said variant but not having no deletions or relative to the parent alpha-amylase or to an alpha-amylase with SEQ ID NO 4 when measured in the Megazyme assay as described in the Examples.

Thus one embodiment concerns the use in laundry, dish wash, industrial or institutional cleaning of a variant or a variant of a parent alpha-amylase wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and wherein the variant alpha-amylase having a degree of identity to the mature polypeptide of SEQ ID NO 3, 4, 5 or 6 of at least 60%, preferred at least 65%, preferred at least 70%, preferred at least 75% preferred at least 80%, preferred at least 81%, preferred at least 82%, preferred at least 83%, preferred at least 84%, preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% and wherein the activity of the said amylase variant at 20 deg C. and at pH 8 is at least 10%, such as at least 20%, preferably at least 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% increased relative to an alpha-amylase having the identical amino acid sequence of said variant but not having the deletion at two or more of said positions or relative to the parent alpha-amylase or to an alpha-amylase with SEQ ID NO 4 when measured in the Megazyme assay as described in the Examples.

Another embodiment concerns the use in laundry, dish wash, industrial or institutional cleaning of a variant or a variant of a parent alpha-amylase wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase having a degree of identity to the mature polypeptide of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%, and wherein the activity of the said amylase variant at 20 deg C. and at pH 8 is at least 10%, such as at least 20%, preferably at least 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% increased relative to an alpha-amylase having the identical amino acid sequence of said variant but not having the deletion at two or more of said positions nor the further substitutions or relative to the parent alpha-amylase or an alpha-amylase with SEQ ID NO 4 when measured in the Megazyme assay as described in the Examples.

A preferred aspect of the invention concerns the use in laundry, dish wash, industrial or institutional cleaning of a variant or a variant of a parent alpha-amylase, wherein the variant comprise a deletion at one or more positions corresponding to positions selected from the group consisting of 180, 181, 182, 183, and 184 of the mature polypeptide of SEQ ID NO 3, and wherein the increase in activity of the amylase variant at 20 deg C. and at pH 8 is at least 10%, such as at least 20%, preferably at least 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% relative to an alpha-amylase having the identical amino acid sequence of said variant but not having the deletion at one or more of said positions, or relative to the parent alpha-amylase or relative to an alpha-amylase with SEQ ID NO 4, and wherein the increase in activity of the same variants at 50 deg C. and at pH 8 is at least 5%, such as at least 10%, such as at least 15%, such as at least 20%, such as at least 25%, such as at least 30% relative to an alpha-amylase having the identical amino acid sequence of said variant but not having the deletion at one or more of said positions or relative to the parent alpha-amylase or relative to an alpha-amylase with SEQ ID NO 4 when measured in the Megazyme assay.

A preferred aspect of the invention concerns the use in laundry, dish wash, industrial or institutional cleaning of a variant or a variant of a parent alpha-amylase, wherein the variant comprise a deletion at one or more positions corresponding to positions selected from the group consisting of 180, 181, 182, 183, and 184 of the mature polypeptide of SEQ ID NO 3, and wherein the increase in activity of the amylase variant at 20 deg C. and at pH 10 is at least 10%, such as at least 20%, preferably at least 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% relative to the parent alpha-amylase or relative to an alpha-amylase with SEQ ID NO 4 and wherein the increase in activity of the same variants at 50 deg C. and at pH 10 is at least 5%, such as at least 10%, such as at least 15%, such as at least 20%, such as at least 25%, such as at least 30% relative to an alpha-amylase having the identical amino acid sequence of said variant but not having the deletion at one or more of said positions or relative to the parent alpha-amylase or relative to an alpha-amylase with SEQ ID NO 4 when measured in the Megazyme assay.

Having an amylase enzyme with high activity both at 20 and 50 deg C. is particularly interesting since when formulated into detergent composition, e.g. for use in wash, it is not necessary to use e.g. wash powder especially adapted for cold wash. Thus in one aspect of the invention the amylase variant is used for wash at a broad temperature range such as 5-65 deg C., such as 5-50 deg C., 5-40 deg C., 5-37 deg C., 5-35 deg C., 5-30 deg C., 5-25 deg C., 5-20 deg C., 5-15 deg C., 5-10 deg C., 10-20 deg C., 10-40 deg C., 10-37 deg C., 10-35 deg C., 10-30 deg C., 10-20 deg C., 15-40 deg C., 15-20 deg C., 15-30 deg C., 20-50 deg C. or 20-55 deg C.

In another aspect the variant of the invention has increased or preserved its stability compared to the parent. This means that the variant either is as stable as the parent or has improved stability. Thus one aspect concerns the use in a starch removing process wherein the residual activity of the variant is at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 deg C., wherein the activity is determined by the method described in Methods section.

Another aspect relates to the use in laundry, dish wash, industrial or institutional cleaning wherein the residual activity of the variant according to the invention is at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 deg C., wherein the activity is determined by the method described in Methods section.

Another aspect relates to the use in laundry, dish wash, industrial or institutional cleaning of an alpha-amylase variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and wherein the variant alpha-amylase has a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% and wherein the residual activity of the variant is at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 deg C., wherein the activity is determined by the method described in Example section.

Another aspect relates to the use in laundry, dish wash, industrial or institutional cleaning of an alpha-amylase variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase has a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% and wherein the residual activity of the variant is at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 deg C., wherein the activity is determined by the method described in Methods section.

Surprisingly the stabilizing effect was also observed when calcium concentration was low (or absent). Thus in one aspect of the invention the variant or the variant of a parent alpha-amylase has at least 30%, such as at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% residual activity after 2 weeks at 37 □deg C. when calcium is low or absent, wherein the activity is determined by the method described in the Methods section below.

It is known that the stability of an enzyme can be improved by shortening of a flexible loop. This can be explained by forming a less flexible protein molecule where parts of the peptide chain is less exposed to proteolytic attack and the three dimensional structure is more rigid thereby reducing the risk of spontaneous unfolding (denaturation) at a given set of conditions. However, in order to perform a catalytic event, some flexibility of the enzyme is needed. Hence, deletions involving shortening of flexible loops are often associated with a reduced activity and performance at reduced temperatures (Georlette et al, 2004; Feller et al. 2003). However, the inventors have found that the deletions around the position X182 both leads to an increased stability in a detergent and to an increase in activity and performance e.g. wash performance, at low temperatures compared to an alpha-amylase having the identical amino acid sequence of said variant but not having the deletion at one or more of said specified positions, or compared to the parent molecule or compared to an alpha-amylase with SEQ ID NO 4.

Thus in a preferable aspect of the invention the variant of the present invention has improved wash performance at low temperature, compared to an alpha-amylase having the identical amino acid sequence of said variant but not having any alterations or relative to the parent or compared to an alpha-amylase with SEQ ID NO 4. Thus one aspect of the invention concerns the use in laundry, dish wash, and industrial or institutional cleaning of a variant or a variant of a parent alpha-amylase, comprising a deletion at one or more positions corresponding to positions selected from the group consisting of 181, 182, 183 and 184 of the mature polypeptide of SEQ ID NO 3, wherein the variant has improved wash performance at 20 deg C. compared to an alpha-amylase having the identical amino acid sequence of said variant but not having the deletion at one or more of said positions or compared to the parent alpha-amylase or compared to an alpha-amylase having the amino acid sequence shown in SEQ ID NO 4 when measured e.g. in AMSA as described herein.

In a preferred embodiment the wash performance of the variant according to the invention at 20 deg C. is at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180% compared to an alpha-amylase having the identical amino acid sequence of said variant but not having the deletion at one or more of said positions or compared to the parent or compared to an alpha amylase with SEQ ID NO 4 when measured e.g. in AMSA as described herein.

Thus one aspect concerns the use in laundry, dish wash, industrial or institutional cleaning of an alpha-amylase variant, wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% and wherein the wash performance of the variant at 20 deg C. is at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180% compared to an alpha-amylase having the identical amino acid sequence of said variant but not having the deletion at two or more of said positions or compared to the parent or compared to an alpha-amylase with SEQ ID NO 4.

The present inventors surprising found that the use of the variants according to the invention at low washing temperatures did not require an increase of the dosage of the variant enzyme needed relative to the traditional dosage used at washing temperatures at or above 40 deg C. Thus, in an interesting embodiment, the use in laundry, dish wash, industrial or institutional cleaning is one wherein the variant is used at dosage ranges selected from the group consisting of 0.02 to 1.2 mg/L, 0.02 to 1.0 mg/L, 0.02 to 0.5 mg/L, 0.02 to 0.4 mg/L, 0.1 to 1.2 mg/L, 0.1 to 1.0 mg/L, 0.1 to 0.5 mg/L, 0.05 to 1.2 mg/L, 0.05 to 1.0 mg/L, 0.05 to 0.5 mg/L, and 0.02 to 2 mg/L.

Another aspect concerns the use in laundry, dish wash, industrial or institutional cleaning of an alpha-amylase variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%, and wherein the wash performance of the variant at 20 deg C. is at least at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180% compared to an alpha-amylase having the identical amino acid sequence of said variant but not having the deletion at two or more of said positions nor having further substitutions or compared to the parent or compared to an alpha-amylase with SEQ ID NO 4.

Thus in a preferred aspect of the invention the wash performance of the variant is increased at temperatures below 40 deg C., such as below 35 deg C., preferably by at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180% compared to an alpha-amylase having the identical amino acid sequence of said variant but not having no deletion nor substitutions or compared to the parent or compared to an alpha-amylase with SEQ ID NO 4.

In another preferred aspect of the invention the wash performance of the variant is increased compared to an alpha-amylase having the identical amino acid sequence of said variant but having neither deletion nor substitutions or compared to the parent alpha-amylase at temperatures below 40 deg C., preferably below 35 deg C., preferably below 30 deg C., preferably below 25 deg C., preferably below 20 deg C., preferably below 15 deg C., preferably below 10 deg C. or even more preferably below 5 deg C.

In another preferred aspect of the invention the wash performance of the variant is increased compared to an alpha-amylase having the identical amino acid sequence of said variant but having neither deletion nor substitution or compared to the parent alpha-amylase at temperatures in the range of 5-40 deg C. In further preferred embodiments, the wash performance of the variant is increased compared to an alpha-amylase having the identical amino acid sequence of said variant but having neither deletion nor substitutions or compared to the parent alpha-amylase or compared to an alpha-amylase having the amino acid sequence shown in SEQ ID NO 4 at temperatures in the range of 5-65 deg C., 5-50 deg C., 5-40 deg C., 5-37 deg C., 5-35 deg C., 5-30 deg C., 5-25 deg C., 5-20 deg C., 5-15 deg C., 5-10 deg C., 10-20 deg C., 10-37 deg C., 10-35 deg C., 10-30 deg C., 10-20 deg C., 15-40 deg C., 15-20 deg C., 15-30 deg C., 20-50 deg C. or 20-55 deg C.

Further aspects of the present invention relate to the use in laundry, dish was, industrial or institutional cleaning of one of the following alpha-amylase variants, and wherein the temperature in the laundry, dish was, industrial or institutional cleaning is one of the following:

| Variant comprising an amino | |
|---|---|
| acid sequence having at least | |
| 60% identity to an amino acid | |
| sequence from the group | |
| consisting of SEQ ID NO 3, | |
| SEQ ID NO 4, SEQ ID NO 5 | |
| and SEQ ID NO 6 and having | Cleaning process is performed at |
| one of the following mutations: | a temperature |
| 180* | below 40 deg C., below 39 deg C., |
| 181* | below 38 deg C., below 37 deg C., |
| 182* | below 36 deg C., below 35 deg C., |
| 183* | below 34 deg C., below 33 deg C., |
| 184* | below 32 deg C., below 31 deg C., |
| 180* + 181* | below 30 deg C., below 29 deg C., |
| 181* + 182* | below 28 deg C., below 27 deg C., |
| 181* + 184* | below 26 deg C., below 25 deg C., |
| 182* + 184* | below 24 deg C., below 23 deg C., |
| 181* + 183* | below 22 deg C., below 21 deg C., |
| 182* + 183* | below 20 deg C., below 19 deg C., |
| 183* + 184* | below 18 deg C., below 17 deg C., |
| | below 16 deg C., below 15 deg C., |
| | below 14 deg C., below 13 deg C., |
| | below 12 deg C., below 11 deg C., |
| | below 10 deg C., below 9 deg C., |
| | below 8 deg C., below 7 deg C., below |
| | 6 deg C., below 5 deg C., below 4 deg |
| | C., below 3 deg C., below 2 deg C., |
| | below 1 deg C. |

It will be understood from the above table, at each of the variants according to the invention has at least one improved property relative to an alpha-amylase having the identical amino acid sequence of said variant but not having the deletion at one or more of said positions or relative to the parent alpha-amylase or relative to an alpha-amylase having the amino acid sequence shown in SEQ ID NO 4.

In a particular preferred embodiment of the invention concerns the use in laundry, dish wash, industrial or institutional cleaning of a variant or a variant of a parent alpha-amylase, wherein the variant has at least two improved properties selected from increased activity, increased wash performance and increased stability.

Thus one aspect of the invention relates to the use in laundry, dish wash, industrial or institutional cleaning of a variant or a variant according to the invention, wherein the variant has increased activity relative to an alpha-amylase having the identical amino acid sequence of said variant but having neither a deletion nor a substitutions or relative to the parent alpha-amylase or relative to an alpha-amylase with SEQ ID NO 4 at 20 deg C., and pH 8 of at least 10%, such as at least 20%, preferably at least 30% such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% increased relative to an alpha-amylase having the identical amino acid sequence of said variant but having neither a deletion nor a substitution or relative to the parent alpha-amylase or relative to an alpha-amylase with SEQ ID NO 4 when measured in the Megazyme assay as described in the Examples, and wherein the variant has an increased wash performance relative to an alpha-amylase having the identical amino acid sequence of said variant but having neither a deletion nor a substitution or relative to the parent alpha-amylase or relative to an alpha-amylase with SEQ ID NO 4 at 20 deg C. of at least 10% at least at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180%.

Another aspect concerns the use in laundry, dish wash, industrial or institutional cleaning of a variant according to the invention wherein the variant has increased activity relative to an alpha-amylase having the identical amino acid sequence of said variant but having neither a deletion nor a substitution or relative to the parent alpha-amylase at 20 deg C., and pH 8 of at least 10%, such as at least 20%, preferably least 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% increase relative to an alpha-amylase having the identical amino acid sequence of said variant but having a deletion nor a substitution or relative to the parent alpha-amylase or relative to an alpha-amylase with SEQ ID NO 4 when measured in the Megazyme assay as described in the Methods section below; and wherein the variant has at least 40% residual activity, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37□deg C., the activity is determined by the method described in Methods section after 2 weeks at 37 □deg C.

A further aspect concerns the use in laundry, dish wash, industrial or institutional cleaning of a variant or a variant according to the invention, wherein the variant has an increased wash performance of at least 10% at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180% compared to an alpha-amylase having the identical amino acid sequence of said variant but having neither a deletion nor a substitution or relative to the parent or compared to an alpha-amylase with SEQ ID NO 4 and wherein the variant has at least 40% residual activity, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 deg C. when the activity is determined by the method described in Methods section relative to an alpha-amylase having the identical amino acid sequence of said variant but having neither a deletion nor a substitution or relative the parent alpha-amylase or relative to an alpha-amylase with SEQ ID NO 4.

A particular preferred embodiment of the invention concerns the use in laundry, dish wash, industrial or institutional cleaning of a variant according to the invention wherein the variant has increased activity relative to an alpha-amylase having the identical amino acid sequence of said variant but having neither a deletion nor a substitution or relative to the parent alpha-amylase at 20 deg C., and pH 8 of at least 10%, such as at least 20%, preferably 30%, such as at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% increase relative to an alpha-amylase having the identical amino acid sequence of said variant but having neither a deletion nor a substitution or relative to the parent alpha-amylase or relative to an alpha-amylase with SEQ ID NO 4 when measured in the Megazyme assay as described in the Method section, and wherein the variant has an increased wash performance relative to the parent alpha-amylase or relative to an alpha-amylase with SEQ ID NO 4 at 20 deg C. of at least 10% at least at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180%, and the variant has at least 40% residual activity, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37□deg C. when the activity is determined by the method described in Example section.

In the context of the present invention, “improved performance” as used in connection with in laundry, dish wash, industrial or institutional cleaning, as already indicated above, relates to improved removal of starchy stains, i.e. stains containing starch, during washing or dishwashing, respectively. The performance may be determined in conventional washing and dishwashing experiments and the improvement evaluated as a comparison with the performance of an alpha-amylase having the identical amino acid sequence of said variant but having neither a deletion nor a substitution or with the performance of the parent alpha-amylase in question or with the performance of an alpha-amylase having the amino acid sequence shown in SEQ ID NO 4. The wash performance may e.g. be measured in an AMSA assay as described in the Methods section.

It will be understood that a variety of different characteristics of an alpha-amylase variant, including specific activity, substrate specificity, Kₘ (the so-called “Michaelis constant” in the Michaelis-Menten equation), Vₘₐₓ [the maximum rate (plateau value) of conversion of a given substrate determined on the basis of the Michaelis-Menten equation], pl, pH optimum, temperature optimum, thermoactivation, stability towards chelants, oxidants or surfactants (e.g. in detergents), etc., taken alone or in combination, can contribute to improved performance. The skilled person will be aware that the performance of the variant cannot, alone, be predicted on the basis of the above characteristics, but would have to be accompanied by washing and/or dishwashing performance tests.

In further aspects the invention relates to a DNA construct comprising a DNA sequence encoding an alpha-amylase variant of the invention, a recombinant expression vector carrying the DNA construct, a cell which is transformed with the DNA construct or the vector, as well as a method of producing an alpha-amylase variant by culturing such a cell under conditions conducive to the production of the alpha-amylase variant, after which the alpha-amylase variant is recovered from the culture.

In a further aspect the invention relates to a method of preparing a variant of a parent alpha-amylase according to the invention which by virtue of its improved properties as described above exhibits improved properties as compared to an alpha-amylase having the identical amino acid sequence of said variant but having neither a deletion nor a substitution or compared to the parent alpha-amylase or compared to an alpha-amylase with SEQ ID NO 4. This method comprises a) constructing a population of cells containing genes encoding variants or variants of said parent alpha-amylase according to the invention, b) screening the population of cells for alpha-amylase activity under conditions simulating at least one washing and/or dishwashing condition, c) isolating a cell from the population containing a gene encoding a variant or a variant of said parent alpha-amylase which has improved activity as compared to an alpha-amylase having the identical amino acid sequence of said variant but having no deletions or compared to the parent alpha-amylase under the conditions selected in step b), d) culturing the cell isolated in step c) under suitable conditions in an appropriate culture medium, and e) recovering the alpha-amylase variant from the culture obtained in step d).

The invention also relates to a variant (which is a variant according the invention) prepared by the latter method.

In a further aspect the invention relates to a method of doing laundry, dish wash and industrial or institutional cleaning comprising adding to a doing laundry, dish wash and industrial or institutional cleaning process a composition comprising at least one surfactant and a variant or a variant of a parent alpha-amylase, wherein the variant comprises an alteration at one or more, or one or several, positions corresponding to positions selected from the group consisting of 180, 181, 182, 183 and 184, said positions corresponding to one or more positions in SEQ ID NO 3, wherein (a) the alteration(s) are independently, (i) an insertion of an amino acid immediately downstream of the position, (ii) a deletion of the amino acid which occupies the position, and/or (iii) a substitution of the amino acid which occupies the position, (b) the variant has alpha-amylase activity; and (c) said variant having improved activity at low temperature compared to the parent alpha-amylase or compared to an alpha-amylase having the identical amino acid sequence of said variant but not having the alterations at one or more, or one or several, of said positions or compared to an alpha-amylase with SEQ ID NO 4, and wherein said laundry, dish wash and industrial or institutional cleaning is performed at a temperature below 40 deg C., such as below 35 deg C.

An even further aspect of the present invention relates to a method of cleaning comprising adding to a cleaning process a composition comprising at least one surfactant and a variant alpha-amylase, wherein the variant comprises a deletion at one or more, or one or several, positions corresponding to positions selected from the group consisting of 180, 181, 182, 183 and 184 (using the mature polypeptide of SEQ ID NO 3 for numbering), and wherein said variant comprises an amino acid sequence having at least 60% identity to an amino acid sequence from the group consisting of SEQ ID NO 1, SEQ ID NO 2, SEQ ID NO 3, SEQ ID NO 4, SEQ ID NO 5 and SEQ ID NO 6, SEQ ID NO 7, SEQ ID NO 8, SEQ ID NO 9, SEQ ID NO 10, and SEQ ID NO 11 and wherein the variant has at least one improved property relative to an alpha-amylase having the identical amino acid sequence of said variant but not having the deletion at one or more, or one or several, of said positions or relative to an alpha-amylase having the amino acid sequence shown in SEQ ID NO 4 and wherein said cleaning process is performed at a temperature below 40 deg C., such as below 35 deg C.

In a preferred embodiment of the above methods, the cleaning process is selected from the group consisting of at least one cleaning step in a laundry, dish wash, institutional and industrial cleaning process.

In an interesting embodiment of the above methods, the cleaning process is one wherein the variant is used at dosage ranges selected from the group consisting of 0.02 to 1.2 mg/L, 0.02 to 1.0 mg/L, 0.02 to 0.5 mg/L, 0.02 to 0.4 mg/L, 0.1 to 1.2 mg/L, 0.1 to 1.0 mg/L, 0.1 to 0.5 mg/L, 0.05 to 1.2 mg/L, 0.05 to 1.0 mg/L, 0.05 to 0.5 mg/L, and 0.02 to 2 mg/L.

In preferred embodiments, the variant comprises an amino acid sequence having at least 65% identity, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% identity to an amino acid sequence from the group consisting of SEQ ID NO 3, SEQ ID NO 4, SEQ ID NO 5 and SEQ ID NO 6.

A preferred aspect of the invention relates to a method of cleaning comprising adding to a cleaning process a composition comprising at least one surfactant and a variant comprising a deletion at one or more positions corresponding to positions selected from the group consisting of 180, 181, 182, 183 and 184 (using the mature polypeptide of SEQ ID NO 3 for numbering), and wherein said variant comprises an amino acid sequence having at least 60% identity to amino acid sequence from the group consisting of SEQ ID NO 3, SEQ ID NO 4, SEQ ID NO 5 and SEQ ID NO 6, wherein said variant having at least one improved property relative to an alpha-amylase having the identical amino acid sequence of said variant but not having the deletion at one or more of said positions or relative to an alpha-amylase having the amino acid sequence shown in SEQ IN NO 4, and wherein said cleaning is performed at a temperature below 40 deg C., such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C and/or below, such as 10 deg C. and/or below, such as 5 deg C. and/or below, such as 2 deg C. and/or below or 1 deg C. and/or below.

Another preferred aspect of the invention relates to a method of cleaning comprising adding to a cleaning process a composition comprising at least one surfactant and a variant comprising a combination of deletions of two or more positions corresponding to positions 180*+181*, 181*+182*; 181*+183*, 182*+183*; 181*+184*, 182*+184* or 183*+184* (using the mature polypeptide of SEQ ID NO 3 for numbering) and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% identity, more preferred at least 96%, more preferred at least 97%, more preferred at least 98% identity, wherein said variant having at least one improved property relative to an alpha-amylase having the identical amino acid sequence of said variant but not having the deletion at one or more of said positions or to an alpha-amylase having the amino acid sequence shown in SEQ ID NO 4, selected from the group comprising increased activity, increased stability or increased wash performance and wherein said cleaning is performed at a temperature below 40 deg C., such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

Another preferred aspect of the invention relates to a method of doing laundry, dish wash or industrial cleaning comprising adding a composition comprising surfactant and a variant comprising a combination of deletions of two or more positions corresponding to positions 180*+181*, 181*+182*; 181*+183*; 182*+183*; 181*+184*, 182*+184* or 183*+184* (using the mature polypeptide of SEQ ID NO 3 for numbering) and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase has a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%, and wherein said variant having at least one improved property relative to an alpha-amylase having the identical amino acid sequence of said variant but not having the deletion at one or more of said positions or to an alpha-amylase having the amino acid sequence shown in SEQ ID NO 4, selected from the group comprising increased activity, increased stability or increased wash performance, and wherein said I cleaning is performed at a temperature below 40 deg C., such as below 39 deg C., such as below 38 deg C., such as below 37 deg C., such as below 36 deg C., such as below 35 deg C., such as below 34 deg C., such as below 33 deg C., such as below 32 deg C., such as below 31 deg C., such as below 30 deg C., such as below 29 deg C., such as below 28 deg C., such as below 27 deg C., such as below 26 deg C., such as below 25 deg C., such as below 24 deg C., such as below 23 deg C., such as below 22 deg C., such as below 21 deg C., such as below 20 deg C., such as below 19 deg C., such as below 18 deg C., such as below 17 deg C., such as below 16 deg C., such as below 15 deg C., such as below 14 deg C., such as below 13 deg C., such as below 12 deg C., such as below 11 deg C. such as below 10 deg C., such as below 9 deg C., such as below 8 deg C., such as below 7 deg C., such as below 6 deg C., such as below 5 deg C., such as below 4 deg C., such as below 3 deg C., such as below 2 deg C., such as below 1 deg C. In useful embodiments, the temperature is 40 deg C. and/or below, such as 35 deg C. and/or below, such as 32 deg C. and/or below, such as 30 deg C. and/or below, such as 25 deg C. and/or below, such as 20 deg C. and/or below, such as 15 deg C. and/or below, such as 10 deg C. and/or below, such as 5 deg C. and/or below, such as 2 deg C. and/or below or 1 deg C. and/or below.

Further specific embodiments of the method of cleaning according to the invention, include:

| Variant comprising an amino | |
|---|---|
| acid sequence having at least | |
| 60% identity to an amino acid | |
| sequence from the group | |
| consisting of SEQ ID NO 3, | |
| SEQ ID NO 4, SEQ ID 5 and | |
| SEQ ID NO 6 and having | Cleaning process is performed at |
| one of the following mutations: | a temperature |
| 180* | below 40 deg C., below 39 deg C., |
| 181* | below 38 deg C., below 37 deg C., |
| 182* | below 36 deg C., below 35 deg C., |
| 183* | below 34 deg C., below 33 deg C., |
| 184* | below 32 deg C., below 31 deg C., |
| 180* + 181* | below 30 deg C., below 29 deg C., |
| 181* + 182* | below 28 deg C., below 27 deg C., |
| 181* + 184* | below 26 deg C., below 25 deg C., |
| 182* + 184* | below 24 deg C., below 23 deg C., |
| 181* + 183* | below 22 deg C., below 21 deg C., |
| 182* + 183* | below 20 deg C., below 19 deg C., |
| 183* + 184* | below 18 deg C., below 17 deg C., |
| | below 16 deg C., below 15 deg C., |
| | below 14 deg C., below 13 deg C., |
| | below 12 deg C., below 11 deg C., |
| | below 10 deg C., below 9 deg C., |
| | below 8 deg C., below 7 deg C., below |
| | 6 deg C., below 5 deg C., below 4 deg |
| | C., below 3 deg C., below 2 deg C., |
| | below 1 deg C. |

It will be understood from the above table, at each of the variants according to the invention has at least one improved property relative to an alpha-amylase having the identical amino acid sequence of said variant but not having the deletion at one or more of said positions or to an alpha-amylase having the amino acid sequence shown in SEQ ID NO 4.

Another preferred aspect concerns the above method, wherein the cleaning, such as e.g. laundry, dish wash industrial or institutional cleaning, is performed at temperature ranges selected from 10-40 deg C., 10-40 deg C., 15-40 deg C., 15-35 deg C., 15-30 deg C., 5-35 deg C., 5-32 deg C., 5-30 deg, 5-25 deg C., 5-20 deg C., 5-15 deg C., 5-10 deg C., 10-20 deg C., 10-32 deg C., 10-30 deg C., 10-20 deg C., 15-20 deg C.

Another preferred aspect concerns the use of a variant according to the invention wherein the laundry, dish wash, industrial or institutional cleaning is performed at temperature in the range of 5-40 deg C., 15-35 deg C., 15-30 deg C., 5-35 deg C., 5-32 deg C., 5-30 deg, 5-25 deg C., 5-20 deg C., 5-15 deg C., 5-10 deg C., 10-20 deg C., 10-32 deg C., 10-30 deg C., 10-20 deg C., 15-20 deg C.

The present invention also concerns compositions. The term “composition” or “detergent composition” is intended to include one, or a combination of two or more, constituents of the detergent composition in question. The constituents of a number of different detergent compositions are listed further below.

Thus a particular aspect of the invention relates to a composition comprising a surfactant and a variant or a variant of a parent alpha-amylase wherein the variant comprises a deletion at one or more, or one or several, positions corresponding to positions selected from the group consisting of 180, 181, 182, 183 and 184 of the mature polypeptide of SEQ ID NO: 3, wherein the variant has at least one improved property relative to an alpha-amylase having the identical amino acid sequence of said variant but not having the deletion at one or more of said positions or relative to the parent alpha-amylase or relative to an alpha-amylase with SEQ ID NO 4 wherein the improved property is selected from the group consisting of improved activity, improved wash performance and improved stability.

In a preferred embodiment the composition comprises a variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98 and wherein the variant has increased activity relative to an alpha-amylase having the identical amino acid sequence of said variant but not having the deletion at two or more of said positions or relative to the parent alpha-amylase or relative to an alpha-amylase with SEQ ID NO 4, at 20 deg C. and pH 8 of at least 10%, such as at least 20%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%.

Another preferred aspect relates to a composition comprising a variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98, and wherein the variant has increased activity relative to an alpha-amylase having the identical amino acid sequence of said variant but not having the deletion at two or more of said positions nor having the further substitutions or relative to the parent alpha-amylase or relative to an alpha-amylase with SEQ ID NO 4 at 20 deg C. and pH 8 of at least 10%, such as at least 20%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%.

Another aspect of the invention relates to a composition comprising a surfactant and a variant or a variant of an alpha-amylase wherein the variant has an increased wash performance relative to an alpha-amylase having the identical amino acid sequence of said variant but not having any alterations or relative to the parent alpha-amylase or relative to an alpha-amylase with SEQ ID NO 4, at 20 deg C. of at least 10% such as at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180%.

Another preferred aspect relates to a composition comprising a variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% and wherein the variant has an increased wash performance relative to an alpha-amylase having the identical amino acid sequence of said variant but not having the deletion at two or more of said positions or relative to the parent alpha-amylase or relative to an alpha-amylase with SEQ ID NO 4 at 20 deg C. of at least 10% such as at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180%.

In an interesting embodiment of the compositions according to the invention, the composition comprises the variant at dosage ranges selected from the group consisting of 0.02 to 1.2 mg/L, 0.02 to 1.0 mg/L, 0.02 to 0.5 mg/L, 0.02 to 0.4 mg/L, 0.1 to 1.2 mg/L, 0.1 to 1.0 mg/L, 0.1 to 0.5 mg/L, 0.05 to 1.2 mg/L, 0.05 to 1.0 mg/L, 0.05 to 0.5 mg/L, and 0.02 to 2 mg/L.

Another preferred aspect relates to a composition comprising a variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% and wherein the variant has an increased wash performance relative to an alpha-amylase having the identical amino acid sequence of said variant but not having the deletion at two or more of said positions nor having the further substitutions, or relative to the parent alpha-amylase or relative to an alpha-amylase with SEQ ID NO 4 at 20 deg C. of at least 10% such as at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180%.

Another aspect of the invention relates to a composition comprising a surfactant and a variant or a variant of an alpha-amylase, wherein the variant has an increased stability of at least 40% at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 deg C. relative to an alpha-amylase having the identical amino acid sequence of said variant but not having any deletions or substitutions, or relative to the parent alpha-amylase or relative to an alpha-amylase with SEQ ID NO 4.

Another preferred aspect relates to a composition comprising a variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% wherein the variant has an increased stability of at least 40% at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 □deg C relative to the parent alpha-amylase or relative to an alpha-amylase with SEQ ID NO 4.

Another preferred aspect relates to a composition comprising a variant wherein the variant comprises a combination of deletions of two or more positions corresponding to positions 180*+181*, 181*+182*; 181*+183*; 182*+183*; 181*+184*, 182*+184* or 183*+184* of the mature polypeptide of SEQ ID NO 3 and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98% wherein the variant has an increased stability of at least 40% at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 □deg C. relative to an alpha-amylase having the identical amino acid sequence of said variant but not having the deletion at two or more of said positions nor having further substitutions, or relative to the parent alpha-amylase or relative to an alpha-amylase with SEQ ID NO 4.

The invention also relates to a composition comprising a surfactant and variant according to the invention wherein the variant comprises a deletion at one or more positions corresponding to positions selected from the group consisting of 180, 181, 182, 183 and 184 of the mature polypeptide of SEQ ID NO 3, wherein the variant has at least two improved properties relative to an alpha-amylase having the identical amino acid sequence of said variant but not having the deletion at one or more of said positions, or relative to the parent alpha-amylase or relative to an alpha-amylase with SEQ ID NO 4 selected from the group consisting of improved activity, improved wash performance and improved stability.

In a particular preferred embodiment the composition comprises a variant according to the invention wherein the variant has increased activity relative to an alpha-amylase having the identical amino acid sequence of said variant but not having any deletions or substitutions as specified, or relative to the parent alpha-amylase or relative to an alpha-amylase with SEQ ID NO 4 at 20 deg C., and pH 8 of at least 10%, such as at least 20%, preferably at least 30%, preferably at least 30%, preferably of at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% and wherein the variant has an increased wash performance relative to an alpha-amylase having the identical amino acid sequence of said variant but not having any deletions or substitutions as specified, or relative to the parent alpha-amylase or relative to an alpha-amylase with SEQ ID NO 4 at 20 deg C. of at least 10%, such as at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180%.

In a particular preferred embodiment the composition comprises a variant according to the invention wherein the variant has increased activity relative to an alpha-amylase having the identical amino acid sequence of said variant but not having any deletions or substitutions as specified, or relative to the parent alpha-amylase or relative to an alpha-amylase with SEQ ID NO 4 at 20 deg C., and pH 8 of at least 10%, such as at least 20%, preferably at least 30%, preferably at least 30% preferably of at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% and wherein the variant has an increased stability of at least 40% at least 40% at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 □deg C. relative to an alpha-amylase having the identical amino acid sequence of said variant but not having any deletions or substitutions as specified, or relative to the parent alpha-amylase or relative to an alpha-amylase with SEQ ID NO 4.

In a particular preferred embodiment the composition comprises a variant according to the invention wherein the variant has an increased wash performance relative to an alpha-amylase having the identical amino acid sequence of said variant but not having any deletions or substitutions as specified, or relative to the parent alpha-amylase or relative to an alpha-amylase with SEQ ID NO 4 at 20 deg C. of at least 10%, such as at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180% and the wherein the variant has an increased stability of at least 40% at least 40% at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37□deg C. relative to an alpha-amylase having the identical amino acid sequence of said variant but not having any deletions or substitutions as specified, or relative to the parent alpha-amylase or relative to an alpha-amylase with SEQ ID NO 4.

In a particular preferred embodiment the composition comprises a variant according to the invention wherein the variant has increased activity relative to an alpha-amylase having the identical amino acid sequence of said variant but not having any deletions or substitutions as specified, or relative to the parent alpha-amylase at 20 deg C., and pH 8 of at least 10%, such as at least 20%, preferably at least 30%, preferably at least 30% preferably of at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150% and wherein the variant has an increased wash performance relative to an alpha-amylase having the identical amino acid sequence of said variant but not having any deletions or substitutions as specified, or relative to the parent alpha-amylase at 20 deg C. of at least 10%, such as at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, preferably at least 110%, preferably at least 120%, preferably at least 130%, preferably at least 140% or preferably at least 150%, preferably at least 160%, preferably at least 170%, preferably at least 180% and the wherein the variant has an increased stability of at least 40% at least 40% at least 40% at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably at least 100% after 2 weeks at 37 □deg C. relative to an alpha-amylase having the identical amino acid sequence of said variant but not having any deletions or substitutions as specified, or relative to the parent alpha-amylase or relative to an alpha-amylase with SEQ ID NO 4.

In another preferred embodiment the composition comprises a variant comprising a combination of deletions at two or more positions corresponding to positions F180, R181, G182, D183, G184 of the mature polypeptide of SEQ ID NO: 3 in particular the deletions F180*+R181*; R181*+G182*; R181*+D183*; G182*+D183*; 181*+184*, 182*+184* or D183*+G184*.

Such pairwise deletions may thus be the following:

### F180*+R181*; R181*+G182*; D183*+G184*; R181*+D183*; G182*+D183*; G182*+G184*; and R181*+G184* (SEQ ID NO 3);

### F180*+R181*; R181*+G182*; H183*+G184*; R181*+H183*; G182*+H183*; G182*+G184*; and R181*+G184* (SEQ ID NO 4);

### F180*+R181*; R181*+G182*; D183*+G184*; R181*+D183*; G182*+D183*; G182*+G184*; and R181*+G184* (SEQ ID NO 5);

### F180*+R181*; R181*+G182*; T183*+G184*; R181*+T183*; G182*+T183*; G182*+G184*; and R181*+G184* (SEQ ID NO 6);

or equivalents of these pairwise deletions in another alpha-amylase meeting the requirements of a parent α-amylase in the context of the present invention.

In a particularly preferred embodiment the composition comprises a variant or a variant of a parent alpha-amylase comprising a combination of deletions of two or more positions corresponding to positions selected form the group comprising 180, 181, 182, 183 and 184 of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In a particularly preferred embodiment the composition comprises a variant or a variant of a parent alpha-amylase comprising a combination of deletions of two or more positions corresponding to positions 181*+182*; 181*+183*; 182*+183*; 181*+184*, 182*+184* or 183*+184* of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In yet another aspect the composition comprises a variant or a variant of a parent alpha-amylase comprising a combination of deletions of two or more positions corresponding to position 180, 181, 182, 183, 184 of the mature polypeptide of SEQ ID NO 3 and wherein the variant alpha-amylase has a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In a particularly preferred embodiment the composition comprises a variant or a variant of a parent alpha-amylase comprising a combination of deletions of two or more positions corresponding to positions 181*+182*; 181*+183*; 182*+183*; 181*+184*, 182*+184* or 183*+184* of the mature polypeptide of SEQ ID NO 3 and further comprises a substitution at any, or one or more, of the positions corresponding to positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

Yet another embodiment relates to a composition comprising a variant or a variant of a parent alpha-amylase, wherein the variant comprises a combination of deletions of two or more positions corresponding to positions selected from the group consisting of 180, 181, 182, 183 and 184 of the mature polypeptide of SEQ ID NO 3 and further comprising any, or one or more, of the specific substitutions N28R, S36D, T405, V75I, S83N, T90N, S91A, N94S, R118K, T132S, R142K, S154N, R172K, A186G, N195F, Q280N, N311Q, R320K, S323M, R383K, K400R, S437N, S452T, R458K and T459A using numbering according to SEQ ID NO 3 and wherein the variant alpha-amylase has a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

Yet another embodiment relates to composition comprising a variant or a variant of a parent alpha-amylase, wherein the variant comprises a combination of deletions of two or more positions corresponding to positions 180*+181*; 181*+182*; 181*+183*; 182*+183*; 181*+184*, 182*+184* or 183*+184* of the mature polypeptide of SEQ ID NO 3 and further comprising any of the specific substitutions N28R, S36D, T405, V75I, S83N, T90N, S91A, N94S, R118K, T132S, R142K, S154N, R172K, A186G, N195F, Q280N, N311Q, R320K, S323M, R383K, K400R, S437N, S452T, R458K and T459A using numbering according to SEQ ID NO 3 and wherein the variant alpha-amylase having a degree of identity to the mature polypeptide of SEQ ID NO: 3, 4, 5 or 6 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In a particularly preferred embodiment the composition a variant according to the present invention comprises a combination of deletions at two or more positions selected from the group comprising F180, R181, G182, H183 and G184 of the mature polypeptide of SEQ ID NO: 4 in particular the deletions F180*+G182*; R181*+G182*; R181*+H183*; G182*+H183*, 181*+184*, 182*+184* or H183*+G184*. In yet another aspect the variants of the invention comprises the deletions R181*+G182*; R181*+H183*; G182*+H183*; 181*+184*, 182*+184* or H183*+G184* and further comprises a substitution at any, or one or more, of the positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO: 4. In another aspect the variants of the invention comprises the deletions R181*+G182*; R181*+H183*; G182*+H183*; 181*+184*, 182*+184* or H183*+G184* and further comprises any, or one or more, of the specific substitutions at one or more position(s) N28R, S36D, T405, V75I, S83N, T90N, S91A, N94S, R118K, T132S, R142K, S154N, R172K, A186G, N195F, Q280N, N311Q, R320K, S323M, R383K, K400R, S437N, S452T, R458K and T459A of the mature polypeptide of SEQ ID NO: 4.

In one preferred aspect, the composition comprises a variant alpha-amylase comprising an amino acid sequence having a degree of identity to the mature polypeptide of SEQ ID NO: 4 of at least 60%, preferred at least 65%, preferred at least 70%, preferred at least 75% preferred at least 80%, preferred at least 81%, preferred at least 82%, preferred at least 83%, preferred at least 84% preferred at least 85%, preferred at least 86%, preferred at least 87%, preferred at least 88%, preferred at least 89%, especially preferred at least 90%, especially preferred at least 91%, especially preferred at least 92%, especially preferred at least 93%, especially preferred at least 94%, even especially more preferred at least 95% homology, more preferred at least 96%, more preferred at least 97%, more preferred at least 98%.

In a particular embodiment of the invention the variant comprises the following alterations:

### G182*+D183*+N28R

### G182*+D183*+S36D

### G182*+D183*+T40S

### G182*+D183*+V75I

### G182*+D183*+S83N

### G182*+D183*+T90N

### G182*+D183*+S91A

### G182*+D183*+N94S

### G182*+D183*+R118K

### G182*+D183*+T132S

### G182*+D183*+R142K

### G182*+D183*+S154N

### G182*+D183*+R172K

### G182*+D183*+A186G

### G182*+D183*+N195F

### G182*+D183*+Q280N

### G182*+D183*+N311Q

### G182*+D183*+R320K

### G182*+D183*+S323 M

### G182*+D183*+R383K

### G182*+D183*+K400R

### G182*+D183*+S437N

### G182*+D183*+S452T

### G182*+D183*+R458K

### G182*+D183*+T459A

where each position corresponds to a position of the amino acid sequence shown in SEQ ID NO: 3.

In a particularly preferred embodiment of the invention the variant comprises the following alterations in the alpha-amylase having SEQ ID NO 4:

### G182*+H183*+N28R

### G182*+H183*+S36D

### G182*+H183*+T40S

### G182*+H183*+V75I

### G182*+H183*+S83N

### G182*+H183*+T90N

### G182*+H183*+S91A

### G182*+H183*+N94S

### G182*+H183*+R118K

### G182*+H183*+T1325

### G182*+H183*+R142K

### G182*+H183*+S154N

### G182*+H183*+R172K

### G182*+H183*+A186G

### G182*+H183*+N195F

### G182*+H183*+Q280N

### G182*+H183*+N311Q

### G182*+H183*+R320K

### G182*+H183*+S323M

### G182*+H183*+R383K

### G182*+H183*+K400R

### G182*+H183*+S437N

### G182*+H183*+S452T

### G182*+H183*+R458K

### G182*+H183*+T459A

where each position corresponds to a position of the amino acid sequence shown in SEQ ID NO: 3.

The present invention also relates to an article of manufacture comprising

(a) a container holding a cleaning composition comprising a surfactant and variant of the present invention (as defined herein) and
(b) published material associated with the container informing that the cleaning composition is useful in a cleaning process which is performed at a temperature below 40 deg. C., such as below 35 deg. C.

Examples of published material associated with the container include plastic shrink wrap or other material bonded to the container, a leaflet, a brochure, a flyer, and/or an advertisement bonded to or otherwise associated with the container (e.g., at a point of display, in a visual or verbal advertisement).

The cleaning composition may be a detergent composition in any convenient form, e.g. as powder, granules, paste, liquid, a bar, a tablet, a powder, a granule, a paste, a granular or powder- form all-purpose or a “heavy-duty” washing agent, an especially laundry detergent; a liquid, gel or paste-form all-purpose washing agent, especially the so-called heavy-duty liquid type; a liquid fine-fabric detergent; a hand dishwashing agent or a light duty dishwashing agent, especially those of the high-foaming type; a machine dishwashing agent, including the various tablet, granular, liquid and rinse-aid types for household and institutional use. The composition can also be in unit dose packages, including those known in the art and those that are water soluble, water insoluble and/or water permeable. A liquid detergent may be aqueous, typically containing up to 70% water and 0-30% organic solvent, or nonaqueous.

In another embodiment, the present invention relates to an article of manufacture comprising a container holding a cleaning composition comprising a surfactant and variant of the present invention and published material associated with the container informing that the cleaning composition is useful in a cleaning process which is performed at a temperature below 35 deg. C., wherein the variant comprise a deletion at one or more positions corresponding to positions selected from the group consisting of 180, 181, 182, 183 and 184 (using of the mature polypeptide of SEQ ID NO 3 for numbering), and wherein said variant comprises an amino acid sequence having at least 60% identity to amino acid sequence from the group consisting of SEQ ID NO:4, SEQ ID NO:2, SEQ ID NO:3 and SEQ ID NO:4, and wherein the variant has having at least one improved property relative to the alpha-amylase having the identical amino acid sequence of said variant but not having the deletion at one or more of said positions or relative to an alpha-amylase having the amino acid sequence shown in SEQ ID NO 4.

### Influence of Mutations on Particular Properties

From the results obtained by the present inventors it appears that changes in a particular property, e.g. activity at low temperature, increased wash performance or increased stability, exhibited by a variant relative to the parent alpha-amylase, or relative to an alpha-amylase having the identical amino acid sequence of said variant but not having any deletions or substitutions as specified, in question can to a considerable extent be correlated with the type of, and positioning of, mutation(s) (amino acid substitutions, deletions or insertions) in the variant. It is to be understood, however, that the observation that a particular mutation or pattern of mutations leads to changes in a given property in no way excludes the possibility that the mutation(s) in question can also influence other properties.

In this connection, the term “equivalent position” denotes a position which, on the basis of an alignment of the amino acid sequence of the parent alpha-amylase in question with the “reference” alpha-amylase amino acid sequence in question (for example the sequence shown in SEQ ID NO. 3) so as to achieve juxtapositioning of amino acid residues/regions which are common to both, corresponds most closely to (e.g. is occupied by the same amino acid residue as) a particular position in the reference sequence in question.

### Nucleotide Sequences

### Cloning a DNA Sequence Encoding an Alpha-Amylase

The DNA sequence encoding a parent alpha-amylase may be isolated from any cell or microorganism producing the alpha-amylase in question, using various methods well known in the art. First, a genomic DNA and/or cDNA library should be constructed using chromosomal DNA or messenger RNA from the organism that produces the alpha-amylase to be studied. Then, if the amino acid sequence of the alpha-amylase is known, homologous, labelled oligonucleotide probes may be synthesized and used to identify alpha-amylase-encoding clones from a genomic library prepared from the organism in question. Alternatively, a labelled oligonucleotide probe containing sequences homologous to a known alpha-amylase gene could be used as a probe to identify alpha-amylase-encoding clones, using hybridization and washing conditions of lower stringency.

Yet another method for identifying alpha-amylase-encoding clones would involve inserting fragments of genomic DNA into an expression vector, such as a plasmid, transforming alpha-amylase-negative bacteria with the resulting genomic DNA library, and then plating the transformed bacteria onto agar containing a substrate for alpha-amylase, thereby allowing clones expressing the alpha-amylase to be identified.

Alternatively, the DNA sequence encoding the enzyme may be prepared synthetically by established standard methods, e.g., the phosphoroamidite method described by S. L. Beaucage and M. H. Caruthers (1981) or the method described by Matthes et al. (1984). In the phosphoroamidite method, oligonucleotides are synthesized, e.g., in an automatic DNA synthesizer, purified, annealed, ligated and cloned in appropriate vectors.

Finally, the DNA sequence may be of mixed genomic and synthetic origin, mixed synthetic and cDNA origin or mixed genomic and cDNA origin, prepared by ligating fragments of synthetic, genomic or cDNA origin (as appropriate, the fragments corresponding to various parts of the entire DNA sequence), in accordance with standard techniques. The DNA sequence may also be prepared by polymerase chain reaction (PCR) using specific primers, for instance as described in U.S. Pat. No. 4,683,202 or R. K. Saiki et al. (1988).

### Site-Directed Mutagenesis

Once an alpha-amylase-encoding DNA sequence has been isolated, and desirable sites for mutation identified, mutations may be introduced using synthetic oligonucleotides. These oligonucleotides contain nucleotide sequences flanking the desired mutation sites; mutant nucleotides are inserted during oligonucleotide synthesis. In a specific method, a single-stranded gap of DNA, bridging the alpha-amylase-encoding sequence, is created in a vector carrying the alpha-amylase gene. Then the synthetic nucleotide, bearing the desired mutation, is annealed to a homologous portion of the single-stranded DNA. The remaining gap is then filled in with DNA polymerase I (Klenow fragment) and the construct is ligated using T4 ligase. A specific example of this method is described in Morinaga et al. (1984). U.S. Pat. No. 4,760,025 disclose the introduction of oligonucleotides encoding multiple mutations by performing minor alterations of the cassette. However, an even greater variety of mutations can be introduced at any one time by the Morinaga method, because a multitude of oligonucleotides, of various lengths, can be introduced.

Another method for introducing mutations into alpha-amylase-encoding DNA sequences is described in Nelson and Long (1989). It involves the 3-step generation of a PCR fragment containing the desired mutation introduced by using a chemically synthesized DNA strand as one of the primers in the PCR reactions. From the PCR-generated fragment, a DNA fragment carrying the mutation may be isolated by cleavage with restriction endonucleases and reinserted into an expression plasmid.

### Homology (Sequence Identity)

The parameter “sequence Identity” is used for the relatedness between two amino acid sequences or between two nucleotide sequences. For purposes of the present invention, the degree of sequence identity between two amino acid sequences is determined using the Needleman-Wunsch algorithm (Needleman and Wunsch, 1970) as implemented in the Needle program of the EMBOSS package (EMBOSS: The European Molecular Biology Open Software Suite, Rice et al., 2000, preferably version 3.0.0 or later). The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment).

The homology or sequence identity may alternatively be determined by means of older computer programs known in the art such as GAP provided in the GCG program package. Thus, Gap GCGv8 may be used with the default scoring matrix for identity and the following default parameters: GAP creation penalty of 5.0 and GAP extension penalty of 0.3, respectively for nucleic acidic sequence comparison, and GAP creation penalty of 3.0 and GAP extension penalty of 0.1, respectively, for protein sequence comparison. GAP uses the method of Needleman and Wunsch (1970) to make alignments and to calculate the identity.

A structural alignment between Termamyl and a Termamyl-like alpha-amylase may be used to identify equivalent/corresponding positions in other Termamyl-like alpha-amylases. One method of obtaining said structural alignment is to use “ClustalW” (Larkin et al., 2007).

As described above, the immunological cross reactivity, may be assayed using an antibody raised against, or reactive with, at least one epitope of the relevant Termamyl-like alpha-amylase. The antibody, which may either be monoclonal or polyclonal, may be produced by methods known in the art, e.g., as described by Hudson et al., 1989. The immunological cross-reactivity may be determined using assays known in the art, examples of which are Western Blotting or radial immunodiffusion assay, e.g., as described by Hudson et al., 1989. In this respect, immunological cross-reactivity between the alpha-amylases having the amino acid sequences SEQ ID NOS: 1, 2, 3, 4, 5, 6, 7, 8, 9, 10 or 11 respectively, has been found.

### Hybridization

The oligonucleotide probe used in the characterization of the Termamyl-like alpha-amylase in accordance with property iii) above may suitably be prepared on the basis of the full or partial nucleotide or amino acid sequence of the alpha-amylase in question.

Suitable conditions for testing hybridization involve presoaking in 5×SSC and prehybridizing for 1 hour at ˜40° C. in a solution of 20% formamide, 5×Denhardt's solution, 50 mM sodium phosphate, pH 6.8, and 50 mg of denatured sonicated calf thymus DNA, followed by hybridization in the same solution supplemented with 100 mM ATP for 18 hours at ˜40° C., followed by three times washing of the filter in 2×SSC, 0.2% SDS at 40° C. for 30 minutes (low stringency), preferred at 50° C. (medium stringency), more preferably at 65° C. (high stringency), even more preferably at ˜75° C. (very high stringency). More details about the hybridization method can be found in Sambrook et al., 1989.

In the present context, “derived from” is intended not only to indicate an alpha-amylase produced or producible by a strain of the organism in question, but also an alpha-amylase encoded by a DNA sequence isolated from such strain and produced in a host organism transformed with said DNA sequence. Finally, the term is intended to indicate an alpha-amylase, which is encoded by a DNA sequence of synthetic and/or cDNA origin and which has the identifying characteristics of the alpha-amylase in question. The term is also intended to indicate that the parent alpha-amylase may be a variant of a naturally occurring alpha-amylase, i.e. a variant, which is the result of a modification (insertion, substitution, deletion) of one or more amino acid residues of the naturally occurring alpha-amylase.

### Production of Variant Alpha-Amylases

The variant alpha-amylases of the invention may be produced using methods well known in the area. Generally, DNA sequences encoding the parent alpha-amylase is provided and the desired alteration is generated in the nucleic acid sequence using techniques known in the art.

The generated DNA sequence encoding the desired variant alpha-amylase of the invention is provided with suitable regulatory sequences, such as promoter, terminator, activation sites, ribosome binding sites, polyadenylation sites etc. and introduced into a suitable host cell. Finally the host cell comprising said DNA is grown under conditions leading to expression of the variant alpha-amylase according to the invention.

All these techniques are known in the art and it is within the skills of the average practitioner within the field to prescribe a suitable method for producing a given variant alpha-amylase of the invention using techniques disclosed in well known text books such as Sambrook et al., 1989. Further teachings regarding preparation of variant alpha-amylases can be found in WO 2006002643 A2, which is incorporated by reference, and the skilled person will appreciate that this teaching also applies to the present invention.

### INDUSTRIAL APPLICATION

The variant amylases according to the invention may in principle be used in all industrial application applications where alpha-amylases are used, including but not limited to starch conversion, ethanol production, textile desizing, paper and pulp production, beer making and detergents. These and other applications are well known to the skilled person. Detailed description can be found in WO 2006002643 A2, which is incorporated by reference.

In a further aspect the invention also relates to compositions comprising the variant alpha-amylase of the invention. In one preferred embodiment the composition is a detergent composition. The compositions may be prepared using techniques known in the art for preparing enzyme containing compositions. Teaching relating to detergent compositions may be found tin WO 2006002643 A2 and this applies also to the present invention.

### Detergent Compositions

When an alpha-amylase variant of the invention is employed as a component of a detergent composition (e.g. a laundry washing detergent composition, or a dishwashing detergent composition), it may, for example, be included in the detergent composition in the form of a non-dusting granulate, a stabilized liquid, or a protected enzyme.

As mentioned above, non-dusting granulates may be produced, e.g., as disclosed in U.S. Pat. Nos. 4,106,991 and 4,661,452 (both to Novo Industri A/S) and may optionally be coated by methods known in the art. Examples of waxy coating materials are poly(ethylene oxide) products (polyethyleneglycol, PEG) with mean molecular weights of 1000 to 20000; ethoxylated nonyl-phenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

Enzymes added in the form of liquid enzyme preparations may, as indicated above, be stabilized by, e.g., the addition of a polyol such as propylene glycol, a sugar or sugar alcohol, lactic acid or boric acid according to established methods. Other enzyme stabilizers are well known in the art.

Protected enzymes for inclusion in a detergent composition of the invention may be prepared, as mentioned above, according to the method disclosed in EP 238,216.

The detergent composition of the invention may be in any convenient form, e.g. as powder, granules, paste, liquid, a bar, a tablet, a powder, a granule, a paste, a granular or powder-form all-purpose or a “heavy-duty” washing agent, an especially laundry detergent; a liquid, gel or paste-form all-purpose washing agent, especially the so-called heavy-duty liquid type; a liquid fine-fabric detergent; a hand dishwashing agent or a light duty dishwashing agent, especially those of the high-foaming type; a machine dishwashing agent, including the various tablet, granular, liquid and rinse-aid types for household and institutional use. The composition can also be in unit dose packages, including those known in the art and those that are water soluble, water insoluble and/or water permeable. A liquid detergent may be aqueous, typically containing up to 70% water and 0-30% organic solvent, or nonaqueous.

The composition may be augmented with one or more agents for preventing or removing the formation of the biofilm. These agents may include, but are not limited to, dispersants, surfactants, detergents, other enzymes, anti-microbials, and biocides.

The detergent composition of the present invention may further comprise surfactants, builders, bleaches, bleach catalysts, colorants, bleach boosters, chelating agents, dye transfer agents, deposition aids, dispersants, additional enzymes, and enzyme stabilizers, catalytic materials, bleach activators, hydrogen peroxide, sources of hydrogen peroxide, optical brighteners, photoactivators, fluorescers, fabric conditioners, preformed peracids, polymeric dispersing agents, clay soil removal/anti-redeposition agents, filler salts, hydrotropes, brighteners, suds suppressors, structure elasticizing agents, fabric softeners, hydrolyzable surfactants, preservatives, anti-oxidants, anti-shrinkage agents, germicides, fungicides, anti-tarnish, anti-corrosion agents, alkalinity sources, solubilizing agents, carriers, processing aids, pigments, dyes, perfumes and pH control agents.

Thus, in a preferred embodiment, the composition comprises a surfactant. The surfactant may be a non-ionic including semi-polar and/or anionic and/or cationic and/or zwitterionic surfactant and/or ampholytic and/or semi-polar nonionic and/or mixtures thereof.

Anionic surfactants contemplated include linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

Non-ionic surfactants contemplated include alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

The surfactants may be present at a level of from 0.1% to 60% by weight of the composition, while in alternative embodiments, the level is from about 1 percent to about 50 percent, while in still further embodiments, the level is from about 5 percent to about 40 percent, by weight of the detergent composition.

When included therein the detergent will usually contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

Further suitable anionic surfactants are soaps and those containing sulfate or sulfonate groups. Surfactants of the sulfonate type that come into consideration are (C9-C13-alkyl)benzenesulfonates and olefinsulfonates, the latter being understood to be mixtures of alkenesulfonates and hydroxyalkanesulfonates and -disulfonates, as obtained, for example, by sulfonation of C12-C18 monoolefins having a terminally or internally located double bond. Also suitable are (C12-C18)alkanesulfonates and esters of alpha-sulfo fatty acids (ester sulfonates), for example the alpha-sulfonated methyl esters of hydrogenated coconut, palm kernel or tallow fatty acids, typically produced by saponification of triglycerides from the plant or animal oils followed by methylation and sulfonation, may be used.

Further suitable anionic surfactants are sulfonated fatty acid glycerol esters comprising mono-, di- and tri-esters and mixtures thereof.

Alk(en)yl sulfates to which preference is given are the alkali metal salts and the sodium salts of sulfuric acid monoesters of C12-C18 fatty alcohols, for example from coconut fatty alcohol, tallow fatty alcohol, lauryl, myristyl, cetyl or stearyl alcohol, or of C10-C20 oxo alcohols and sulfuric acid monoesters of secondary alcohols having that chain length. From the point of view of washing technology, special preference is given to C12-C16 alkyl sulfates and C12-C15 alkyl sulfates and also to C14-C15 alkyl sulfates. Suitable anionic surfactants are also alkane-2,3-diylbis(sulfates) that are prepared, for example, in accordance with U.S. Pat. No. 3,234,258 or U.S. Pat. No. 5,075,041.

Also suitable are the sulfuric acid monoesters of straight-chain or branched C7-C21 alcohols ethoxylated with from 1 to 6 mole of ethylene oxide, such as 2-methyl-branched C9-C11 alcohols with, on average, 3.5 mole of ethylene oxide (EO) or C12-C18 fatty alcohols with from 1 to 4 EO. Because of their high foaming characteristics, they are normally used in washing and cleaning compositions only at relatively low levels, for example at levels of from 1% to 5% by weight.

Anionic surfactants may also include diesters, and/or salts of monoesters, of sulfosuccinic acid with C8-C18 fatty alcohol residues or mixtures thereof. Special preference is given to sulfosuccinates in which the fatty alcohol residues have a narrow chain length distribution. It is likewise also possible to use alk(en)yl sulfosuccinates having preferably from 8 to 18 C-atoms in the alk(en)yl chain, or salts thereof.

Further anionic surfactants that come into consideration are fatty acid derivatives of amino acids, for example of methyltaurine (taurides) and/or of methylglycine (sarcosides).

Further anionic surfactants that come into consideration are soaps. Saturated fatty acid soaps such as the salts of lauric acid, myristic acid, palmitic acid, stearic acid, hydrogenated erucic acid and behenic acid and soap mixtures derived from natural fatty acids, for example coconut, palm kernel or tallow fatty acids. The anionic surfactants, including the soaps, may be present in the form of their sodium, potassium or ammonium salts and in the form of soluble salts of organic bases such as mono-, di- or triethanolamine. The anionic surfactants may be present in the form of their sodium or potassium salts. As non-ionic surfactants, preferably alkoxylated, advantageously ethoxylated and/or propoxylated, especially primary alcohols having from 8 to 18 C-atoms and, on average, from 1 to 12 moles of ethylene oxide (EO) and/or from 1 to 10 moles of propylene oxide (PO) per mole of alcohol are used. Special preference is given to C8-C16 alcohol alkoxylates, advantageously ethoxylated and/or propoxylated C10-C15 alcohol alkoxylates, especially C12-C14 alcohol alkoxylates, having a degree of ethoxylation between 2 and 10, or between 3 and 8, and/or a degree of propoxylation between 1 and 6, or between 1.5 and 5. The alcohol residue may be preferably linear or, especially in the 2-position, methyl-branched, or may comprise a mixture of linear and methyl-branched chains, as are usually present in oxo alcohols. Special preference is given, however, to alcohol ethoxylates derived from linear alcohols of natural origin that contain from 12 to 18 C-atoms, for example coconut, palm and tallow fatty alcohol or oleyl alcohol, and on average from 2 to 8 EO per mole of alcohol. The ethoxylated alcohols include, for example, C12-C14 alcohols with 3 EO or 4 EO, C9-C11 alcohols with 7 EO, C13-C15 alcohols with 3 EO, 5 EO, 7 EO or 8 EO, C12-18 alcohols with 3 EO, 5 EO or 7 EO, mixtures thereof, such as mixtures of C12-C14 alcohol with 3 EO and C12-C18 alcohol with 5 EO. The mentioned degrees of ethoxylation and propoxylation represent statistical averages which, for a specific product, can be a whole number or a fractional number. Preferred alcohol ethoxylates and propoxylates have a restricted homologue distribution (narrow range ethoxylates/propoxylates, NRE/NRP). In addition to those non-ionic surfactants, fatty alcohol ethoxylates having more than 12 EO may also be used. Examples thereof are tallow fatty alcohol ethoxylate with 14 EO, 25 EO, 30 EO or 40 EO.

Also suitable are alkoxylated amines, which are ethoxylated and/or propoxylated, especially primary and secondary amines having from 1 to 18 C-atoms per alkyl chain and, on average, from 1 to 12 moles of ethylene oxide (EO) and/or from 1 to 10 moles of propylene oxide (PO) per mole of amine.

In addition, as further non-ionic surfactants, there may also be used alkyl polyglycosides of the general formula R₁O(G)ₓ, wherein R₁ is a primary straight-chain or methyl-branched (especially methyl-branched in the 2-position) alkyl group having from 8 to 22, preferably from 12 to 18, C-atoms and the symbol ‘G’ indicates a glycose (monosaccharide) unit having 5 or 6

C-atoms; preferably G is glucose. The degree of oligomerisation x, which indicates the average number of glycose units, will generally lie between 1 and 10; x is preferably from 1.2 to 1.4.

A further class of used non-ionic surfactants, which are used either as sole non-ionic surfactant or in combination with other non-ionic surfactants, comprises alkoxylated, preferably ethoxylated or ethoxylated and propoxylated fatty acid alkyl esters, having from 1 to 4 C-atoms in the alkyl chain, especially fatty acid methyl esters, as described, for example, in JP58/217598.

Non-ionic surfactants of the amine oxide type, for example N-(coco alkyl)-N,N-dimethylamine oxide and N-(tallow-alkyl)-N,N-bis(2-hydroxyethyl)amine oxide, and of the fatty acid alkanolamide or ethoxylated fatty acid alkanolamide type may also be suitable.

In a more preferred embodiment, the surfactant is sodium dodecyl sulfate, quaternary ammonium compounds, alkyl pyridinium iodides, Tween 80, Tween, 85, Triton X-100, Brij 56, biological surfactants, rhamnolipid, surfactin, visconsin, or sulfonates.

When included therein the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”). In some embodiments the invention relates to a method wherein the concentration of the at least one surfactant is from 0 to 500, from 0.00001 to 100, from 0.0001 to 50, from 0.0001 to 40, from 0.001 to 30, from 0.01 to 20, from 0.1 to 15, from 1 to 10 milligram per gram textile in the wash.

In some embodiments the invention relates to a method, wherein the concentration of the at least one surfactant is from 0 to 50, from 0.0001 to 40, from 0.001 to 30, from 0.01 to 20 from 0.1 to 10, or from 1 to 5 g per L solution.

The detergent composition may additionally comprise one or more other enzymes, such as pullulanase, esterase, lipase, cutinase, protease, cellulase, peroxidase, or oxidase, e.g., laccase or other carbohydrases, such as mannanases, and pectinases. In general the properties of the chosen enzyme(s) should be compatible with the selected detergent, (i.e. pH-optimum, compatibility with other enzymatic and non-enzymatic ingredients, etc.), and the enzyme(s) should be present in effective amounts.

Normally the detergent contains 0-65% of a detergent builder (although some dishwashing detergents may contain even up to 90% of a detergent builder) or complexing agent. In preferred embodiments, the amount of a detergent builder may be above 5%, above 10%, above 20%, above 30%, above 40% or above 50%, and may be below 80%, 65%. In a dishwash detergent, the level of builder is typically 40-65%, particularly 50-65% or even 75-90%

The detergent builders may be subdivided into phosphorus-containing and non-phosphorous-containing types. Examples of phosphorus-containing inorganic alkaline detergent builders include the water-soluble salts, especially alkali metal pyrophosphates, orthophosphates, polyphosphates and phosphonates. Examples of non-phosphorus-containing inorganic builders include water-soluble alkali metal carbonates, borates and silicates, as well as layered disilicates and the various types of water-insoluble crystalline or amorphous alumino silicates of which zeolites are the best known representatives.

Examples of suitable organic builders include alkali metal, ammonium or substituted ammonium salts of succinates, malonates, fatty acid malonates, fatty acid sulphonates, carboxymethoxy succinates, polyacetates, carboxylates, polycarboxylates, aminopolycarboxylates and polyacetyl carboxylates.

The builder may particularly be a chelating agent that forms water-soluble complexes with Ca and Mg. The strength of the complex formed between the builder and Ca⁺⁺ and/or Mg⁺⁺, expressed as the log K value (either given as the equilibrium or stability constant or as the conditional stability constant at a given pH), may be in the range 3-8, particularly 5-8. The stability constant may be measured at 25° C. and ionic strength 0.1M, and the conditional stability constant may be measured at the same conditions at pH 8.5 or 9.

The builder may contain an amino group and may be, e.g., amino carboxylate, amino-polycarboxylate or a phosphonate. It may be a monomeric molecule comprising one, two or three amino groups (typically secondary or tertiary amino groups), and it may contain two, three, four or five carboxyl groups. Examples of suitable builders are methyl glycine diacetic acid (MGDA), glutamic acid N,N-diacetic acid (N,N-dicarboxymethyl glutamic acid tetrasodium salt, GLDA), nitrilotriacetic acid (NTA), diethylene triamine pentaacetic acid (DTPA), ethylenediaminetetraacetic acid (EDTA), Ethylenediamine-N,N′-disuccinic acid (EDDS), N-(1,2-dicarboxyethyl)-D,L-aspartic acid (IDS) and N-(2-hydroxyethyl)iminodiacetic acid (EDG), and salts thereof.

The builder preferably has a buffering capacity (also termed reserve alkalinity) greater than 4 (the number of equivalents of a strong acid required to change the pH of one litre of a buffer solution by one unit, keeping the total amount of the acid and the salt in the buffer constant).

The builder may be an environmentally friendly sequesterant, e.g. as described in WO09/102,854. Suitable environmentally friendly sequesterants include one or more of amino acid-based sequesterants, succinate-based sequesterants, citric acid and salts thereof.

Examples of suitable amino acid based compounds include MGDA (methyl-glycine-diacetic acid), and salts and derivatives thereof and GLDA (glutamic-N,N-diacetic acid) and salts and derivatives thereof. Other suitable builders are described in U.S. Pat. No. 6,426,229. Particular suitable builders include; for example, aspartic acid-N-monoacetic acid (ASMA), aspartic acid-N,N-diacetic acid (ASDA), aspartic acid-N-monopropionic acid (ASMP), iminodisuccinic acid IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts or ammonium salts thereof. In one aspect, GLDA salts and derivatives thereof may be employed.

In one aspect, the tetrasodium salt of GLDA may be employed.

Further examples of suitable builders include N-(hydroxyethyl)-ethylidenediaminetriacetate (HEDTA), diethanolglycine (DEG), 1-Hydroxy Ethylidene-1,1-Diphosphonic Acid (HEDP), diethylenetriaminepentaacetic acid (DTMPA), Diethylenetriamine Penta (Methylene Phosphonic acid) (DTPMP), Ethylene diamine tetra(methylene phosphonic acid) (EDTMPA) and aminotris(methylenephosphonic acid) (ATMP).

Examples of suitable succinate compounds are described in U.S. Pat. No. 5,977,053. In one aspect, suitable succinate compounds include tetrasodium immino succinate.

Builders may be classified by the test described by M. K. Nagarajan et al., 1984, to determine the minimum builder level required to lower the water hardness at pH 10.5 from 200 ppm (as CaCO3) to 10 ppm in a solution of a hypothetical detergent dosed at 0.200 percent, given as the weight percent builder in the hypothetical detergent. Alternatively, the determination may be made at pH 8.5 to reflect the lower pH of typical modern laundry detergents. Using this method at either pH, the required level may be 0-25% (strong), 25-35% (medium) or >35% (weak). More preferred are compositions including strong and medium builders, most preferred are compositions with strong builders.

The builder may be a strong builder such as methyl glycine diacetic acid (MGDA) or N,N-Dicarboxymethyl glutamic acid tetrasodium salt (GLDA); it may be a medium builder such as sodium tri-poly-phosphate (STPP), or it may be a weak builder such as sodium citrate. More preferred are compositions including strong and medium builders, most preferred are compositions with strong builders. Other examples of builders are zeolite, diphosphate, triphosphate, phosphonate, carbonate, nitrilotriacetic acid, ethylenediaminetetraacetic acid (EDTA), diethylenetriaminepentaacetic acid, alkyl- or alkenylsuccinic acid, soluble silicates and layered silicates (e.g. SKS-6 from Hoechst).

The detergent may also be unbuilt, i.e. essentially free of detergent builder.

The detergent may comprise one or more polymers. Examples are carboxymethylcellulose (CMC; typically in the form of the sodium salt), poly(vinylpyrrolidone) (PVP), polyethyleneglycol (PEG), poly(vinyl alcohol) (PVA), polycarboxylates such as polyacrylates, polymaleates, maleic/acrylic acid copolymers and lauryl methacrylate/acrylic acid copolymers.

The detergent composition could also include an additive, a pre-spotter or a booster, which is added to the wash to increase the general cleaning level, some of these additives may also be used as a pre-treatment agent applied to the textile before the washing step.

The detergent composition may contain bleaching agents of the chlorine/bromine-type or the oxygen-type. The bleaching agents may be coated or encapsulated. Examples of inorganic chlorine/bromine-type bleaches are lithium, sodium or calcium hypochlorite or hypobromite as well as chlorinated trisodium phosphate. The bleaching system may also comprise a H₂O₂ source such as perborate or percarbonate which may be combined with a peracid-forming bleach activator such as tetraacetylethylenediamine (TAED) or nonanoyloxybenzenesulfonate (NOBS).

Examples of organic chlorine/bromine-type bleaches are heterocyclic N-bromo and N-chloro imides such as trichloroisocyanuric, tribromoisocyanuric, dibromoisocyanuric and dichloroisocyanuric acids, and salts thereof with water solubilizing cations such as potassium and sodium. Hydantoin compounds are also suitable. The bleaching system may also comprise peroxyacids of, e.g., the amide, imide, or sulfone type.

In dishwashing detergents the oxygen bleaches are preferred, for example in the form of an inorganic persalt, preferably with a bleach precursor or as a peroxy acid compound. Typical examples of suitable peroxy bleach compounds are alkali metal perborates, both tetrahydrates and monohydrates, alkali metal percarbonates, persilicates and perphosphates. Preferred activator materials are TAED or NOBS.

In general, when a bleaching agent is used, the compositions of the present invention may comprise from about 0.1% to about 50% or even from about 0.1% to about 25% bleaching agent by weight of the subject cleaning composition.

The enzymes of the detergent composition of the invention may be stabilized using conventional stabilizing agents and protease inhibitors, e.g. a polyol such as propylene glycol or glycerol, a sugar or sugar alcohol, different salts such as NaCl and KCl; lactic acid, formic acid, boric acid, or a boric acid derivative such as, e.g., an aromatic borate ester, or a phenyl boronic acid derivative such as 4-formylphenyl boronic acid or a peptide aldehyde such as di-, tri- or tetrapeptide aldehydes or aldehyde analogues (either of the form B1-B0-R wherein, R is H, CH3, CX3, CHX2, or CH2X (X=halogen), B0 is a single amino acid residue (preferably with an optionally substituted aliphatic or aromatic side chain); and B1 consists of one or more amino acid residues (preferably one, two or three), optionally comprising an N-terminal protection group, or as described in WO09118375, WO98/13459) or a protease inhibitor of the protein type such as RASI, BASI, WASI (bifunctional alpha-amylase/subtilisin inhibitors of rice, barley and wheat) or C12 or SSI.

The composition may be formulated as described in, e.g., WO 92/19709 and WO 92/19708 or U.S. Pat. No. 6,472,364. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), Nickel (II), and oxovanadium (IV)).

The enzymes of the invention may also be stabilized by adding reversible enzyme inhibitors, e.g., of the protein type (as described in EP 0 544 777 B1) or the boronic acid type.

The detergent may also contain other conventional detergent ingredients such as, e.g., fabric conditioners including clays, deflocculant material, foam boosters/foam depressors (in dishwashing detergents foam depressors), suds suppressors, anti-corrosion agents, soil-suspending agents, anti-soil-redeposition agents, dyes, dehydrating agents, bactericides, optical brighteners, organic solvents such as ethanol, or perfume.

Typically, the detergent compositions of the present invention comprise at least 0.0001 to about 0.1% weight percent of pure enzyme protein, such as from about 0.0001% to about 0.01%, from about 0.001% to about 0.01% or from about 0.001% to about 0.01%. However, when using a formulated enzyme the detergent composition comprises from about 0.02% to about 20% weight percent, such as or from about 0.05% to about 15% weight, or from about 0.05 to about 20%, or from about 0.05% to about 5%, or from about 0.05% to about 3%.

The pH (measured in aqueous solution at use concentration) will usually be neutral or alkaline, e.g. in the range of 7-11.

Particular forms of laundry detergent compositions within the scope of the invention include:

1) A detergent composition formulated as a granulate having a bulk density of at least 600 g/l comprising

| | |
|---|---|
| Linear alkylbenzenesulfonate (calculated as acid) | 7-12% |
| Alcohol ethoxysulfate (e.g. C₁₂₋₁₈ | 1-4% |
| alcohol, 1-2 EO) or alkyl sulfate (e.g. C₁₆₋₁₈) | |
| Alcohol ethoxylate (e.g. C₁₄₋₁₅ alcohol, 7 EO) | 5-9% |
| Sodium carbonate (as Na₂CO₃) | 14-20% |
| Soluble silicate (as Na₂O, 2SiO₂) | 2-6% |
| Zeolite (as NaAlSiO₄) | 15-22% |
| Sodium sulfate (as Na₂SO₄) | 0-6% |
| Sodium citrate/citric acid (as C₆H₅Na₃O₇/C₆H₈O₇) | 0-15% |
| Sodium perborate (as NaBO₃•H₂O) | 11-18% |
| TAED | 2-6% |
| Carboxymethylcellulose | 0-2% |
| Polymers (e.g. maleic/acrylic acid copolymer, PVP, | 0-3% |
| PEG) | |
| Enzymes (calculated as pure enzyme protein) | 0.0001-0.1% |
| Minor ingredients (e.g. suds suppressors, perfume, | 0-5% |
| optical brightener, photobleach) | |

2) A detergent composition formulated as a granulate having a bulk density of at least 600 g/l comprising

| | |
|---|---|
| Linear alkylbenzenesulfonate (calculated as acid) | 6-11% |
| Alcohol ethoxysulfate (e.g. C₁₂₋₁₈ | 1-3% |
| alcohol, 1-2 EO or alkyl sulfate (e.g. C₁₆₋₁₈) | |
| Alcohol ethoxylate (e.g. C₁₄₋₁₅ alcohol, | 5-9% |
| 7 EO) | |
| Sodium carbonate (as Na₂CO₃) | 15-21% |
| Soluble silicate (as Na₂O, 2SiO₂) | 1-4% |
| Zeolite (as NaAlSiO₄) | 24-34% |
| Sodium sulfate (as Na₂SO₄) | 4-10% |
| Sodium citrate/citric acid (as C₆H₅Na₃O₇/C₆H₈O₇) | 0-15% |
| Carboxymethylcellulose | 0-2% |
| Polymers (e.g. maleic/acrylic acid copolymer, PVP, | 1-6% |
| PEG) | |
| Enzymes (calculated as pure enzyme protein) | 0.0001-0.1% |
| Minor ingredients (e.g. suds suppressors, perfume) | 0-5% |

3) A detergent composition formulated as a granulate having a bulk density of at least 600 g/l comprising

| | |
|---|---|
| Linear alkylbenzenesulfonate (calculated as acid) | 5-9% |
| Alcohol ethoxylate (e.g. C₁₂₋₁₅ alcohol, 7 EO) | 7-14% |
| Soap as fatty acid (e.g. C₁₆₋₂₂ fatty acid) | 1-3% |
| Sodium carbonate (as Na₂CO₃) | 10-17% |
| Soluble silicate (as Na₂O, 2SiO₂) | 3-9% |
| Zeolite (as NaAlSiO₄) | 23-33% |
| Sodium sulfate (as Na₂SO₄) | 0-4% |
| Sodium perborate (as NaBO₃•H₂O) | 8-16% |
| TAED | 2-8% |
| Phosphonate (e.g. EDTMPA) | 0-1% |
| Carboxymethylcellulose | 0-2% |
| Polymers (e.g. maleic/acrylic acid copolymer, PVP, | 0-3% |
| PEG) | |
| Enzymes (calculated as pure enzyme protein) | 0.0001-0.1% |
| Minor ingredients (e.g. suds suppressors, perfume, | 0-5% |
| optical brightener) | |

4) A detergent composition formulated as a granulate having a bulk density of at least 600 g/l comprising

| | |
|---|---|
| Linear alkylbenzenesulfonate (calculated as acid) | 8-12% |
| Alcohol ethoxylate (e.g. C₁₂₋₁₅ alcohol, 7 EO) | 10-25% |
| Sodium carbonate (as Na₂CO₃) | 14-22% |
| Soluble silicate (as Na₂O, 2SiO₂) | 1-5% |
| Zeolite (as NaAlSiO₄) | 25-35% |
| Sodium sulfate (as Na₂SO₄) | 0-10% |
| Carboxymethylcellulose | 0-2% |
| Polymers (e.g. maleic/acrylic acid copolymer, PVP, | 1-3% |
| PEG) | |
| Enzymes (calculated as pure enzyme protein) | 0.0001-0.1% |
| Minor ingredients (e.g. suds suppressors, perfume) | 0-5% |

5) An aqueous liquid detergent composition comprising

| | |
|---|---|
| Linear alkylbenzenesulfonate (calculated as acid) | 15-21% |
| Alcohol ethoxylate (e.g. C₁₂₋₁₅ alcohol, 7 EO or C₁₂₋₁₅ | 12-18% |
| alcohol, 5 EO) | |
| Soap as fatty acid (e.g. oleic acid) | 3-13% |
| Alkenylsuccinic acid (C₁₂₋₁₄) | 0-13% |
| Aminoethanol | 8-18% |
| Citric acid | 2-8% |
| Phosphonate | 0-3% |
| Polymers (e.g. PVP, PEG) | 0-3% |
| Borate (as B₄O₇²⁻) | 0-2% |
| Ethanol | 0-3% |
| Propylene glycol | 8-14% |
| Enzymes (calculated as pure enzyme protein) | 0.0001-0.1% |
| Minor ingredients (e.g. dispersants, suds suppressors, | 0-5% |
| perfume, optical brightener) | |

6) An aqueous structured liquid detergent composition comprising

| | |
|---|---|
| Linear alkylbenzenesulfonate (calculated as acid) | 15-21% |
| Alcohol ethoxylate (e.g. C₁₂₋₁₅ alcohol, 7 EO, or C₁₂₋₁₅ | 3-9% |
| alcohol, 5 EO) | |
| Soap as fatty acid (e.g. oleic acid) | 3-10% |
| Zeolite (as NaAlSiO₄) | 14-22% |
| Potassium citrate | 9-18% |
| Borate (as B₄O₇²⁻) | 0-2% |
| Carboxymethylcellulose | 0-2% |
| Polymers (e.g. PEG, PVP) | 0-3% |
| Anchoring polymers such as, e.g., lauryl methacrylate/ | 0-3% |
| acrylic acid copolymer; molar ratio 25:1; MW 3800 | |
| Glycerol | 0-5% |
| Enzymes (calculated as pure enzyme protein) | 0.0001-0.1% |
| Minor ingredients (e.g. dispersants, suds suppressors, | 0-5% |
| perfume, optical brighteners) | |

7) A detergent composition formulated as a granulate having a bulk density of at least 600 g/l comprising

| | |
|---|---|
| Fatty alcohol sulfate | 5-10% |
| Ethoxylated fatty acid monoethanolamide | 3-9% |
| Soap as fatty acid | 0-3% |
| Sodium carbonate (as Na₂CO₃) | 5-10% |
| Soluble silicate (as Na₂O, 2SiO₂) | 1-4% |
| Zeolite (as NaAlSiO₄) | 20-40% |
| Sodium sulfate (as Na₂SO₄) | 2-8% |
| Sodium perborate (as NaBO₃•H₂O) | 12-18% |
| TAED | 2-7% |
| Polymers (e.g. maleic/acrylic acid copolymer, PEG) | 1-5% |
| Enzymes (calculated as pure enzyme protein) | 0.0001-0.1% |
| Minor ingredients (e.g. optical brightener, suds | 0-5% |
| suppressors, perfume) | |

8) A detergent composition formulated as a granulate comprising

| | |
|---|---|
| Linear alkylbenzenesulfonate (calculated as acid) | 8-14% |
| Ethoxylated fatty acid monoethanolamide | 5-11% |
| Soap as fatty acid | 0-3% |
| Sodium carbonate (as Na₂CO₃) | 4-10% |
| Soluble silicate (as Na₂O, 2SiO₂) | 1-4% |
| Zeolite (as NaAlSiO₄) | 30-50% |
| Sodium sulfate (as Na₂SO₄) | 3-11% |
| Sodium citrate (as C₆H₅Na₃O₇) | 5-12% |
| Polymers (e.g. PVP, maleic/acrylic acid copolymer, | 1-5% |
| PEG) | |
| Enzymes (calculated as pure enzyme protein) | 0.0001-0.1% |
| Minor ingredients (e.g. suds suppressors, perfume) | 0-5% |

9) A detergent composition formulated as a granulate comprising

| | |
|---|---|
| Linear alkylbenzenesulfonate (calculated as acid) | 6-12% |
| Nonionic surfactant | 1-4% |
| Soap as fatty acid | 2-6% |
| Sodium carbonate (as Na₂CO₃) | 14-22% |
| Zeolite (as NaAlSiO₄) | 18-32% |
| Sodium sulfate (as Na₂SO₄) | 5-20% |
| Sodium citrate (as C₆H₅Na₃O₇) | 3-8% |
| Sodium perborate (as NaBO₃•H₂O) | 4-9% |
| Bleach activator (e.g. NOBS or TAED) | 1-5% |
| Carboxymethylcellulose | 0-2% |
| Polymers (e.g. polycarboxylate or PEG) | 1-5% |
| Enzymes (calculated as pure enzyme protein) | 0.0001-0.1% |
| Minor ingredients (e.g. optical brightener, perfume) | 0-5% |

10) An aqueous liquid detergent composition comprising

| | |
|---|---|
| Linear alkylbenzenesulfonate (calculated as acid) | 15-23% |
| Alcohol ethoxysulfate (e.g. C₁₂₋₁₅ alcohol, 2-3 EO) | 8-15% |
| Alcohol ethoxylate (e.g. C₁₂₋₁₅ alcohol, 7 EO, or C₁₂₋₁₅ | 3-9% |
| alcohol, 5 EO) | |
| Soap as fatty acid (e.g. lauric acid) | 0-3% |
| Aminoethanol | 1-5% |
| Sodium citrate | 5-10% |
| Hydrotrope (e.g. sodium toluensulfonate) | 2-6% |
| Borate (as B₄O₇²⁻) | 0-2% |
| Carboxymethylcellulose | 0-1% |
| Ethanol | 1-3% |
| Propylene glycol | 2-5% |
| Enzymes (calculated as pure enzyme protein) | 0.0001-0.1% |
| Minor ingredients (e.g. polymers, dispersants, perfume, | 0-5% |
| optical brighteners) | |

11) An aqueous liquid detergent composition comprising

| | |
|---|---|
| Linear alkylbenzenesulfonate (calculated as acid) | 20-32% |
| Alcohol ethoxylate (e.g. C₁₂₋₁₅ alcohol, 7 EO, or C₁₂₋₁₅ | 6-12% |
| alcohol, 5 EO) | |
| Aminoethanol | 2-6% |
| Citric acid | 8-14% |
| Borate (as B₄O₇²⁻) | 1-3% |
| Polymer (e.g. maleic/acrylic acid copolymer, anchoring | 0-3% |
| polymer such as, e.g., lauryl methacrylate-/acrylic | |
| acid copolymer) | |
| Glycerol | 3-8% |
| Enzymes (calculated as pure enzyme protein) | 0.0001-0.1% |
| Minor ingredients (e.g. hydrotropes, dispersants, | 0-5% |
| perfume, optical brighteners) | |

12) A detergent composition formulated as a granulate having a bulk density of at least 600 g/l comprising

| | |
|---|---|
| Anionic surfactant (linear alkylbenzenesulfonate, alkyl | 25-40% |
| sulfate, alpha-olefinsulfonate, alpha-sulfo fatty acid | |
| methyl esters, alkanesulfonates, soap) | |
| Nonionic surfactant (e.g. alcohol ethoxylate) | 1-10% |
| Sodium carbonate (as Na₂CO₃) | 8-25% |
| Soluble silicates (as Na₂O, 2SiO₂) | 5-15% |
| Sodium sulfate (as Na₂SO₄) | 0-5% |
| Zeolite (as NaAlSiO₄) | 15-28% |
| Sodium perborate (as NaBO₃•4H₂O) | 0-20% |
| Bleach activator (TAED or NOBS) | 0-5% |
| Enzymes (calculated as pure enzyme protein) | 0.0001-0.1% |
| Minor ingredients (e.g. perfume, optical brighteners) | 0-3% |

13) Detergent formulations as described in 1)-12) wherein all or part of the linear alkylbenzenesulfonate is replaced by (C₁₂-C₁₈) alkyl sulfate.
14) A detergent composition formulated as a granulate having a bulk density of at least 600 g/l comprising

| | |
|---|---|
| (C₁₂-C₁₈) alkyl sulfate | 9-15% |
| Alcohol ethoxylate | 3-6% |
| Polyhydroxy alkyl fatty acid amide | 1-5% |
| Zeolite (as NaAlSiO₄) | 10-20% |
| Layered disilicate (e.g. SK56 from Hoechst) | 10-20% |
| Sodium carbonate (as Na₂CO₃) | 3-12% |
| Soluble silicate (as Na₂O, 2SiO₂) | 0-6% |
| Sodium citrate | 4-8% |
| Sodium percarbonate | 13-22% |
| TAED | 3-8% |
| Polymers (e.g. polycarboxylates and PVP) | 0-5% |
| Enzymes (calculated as pure enzyme protein) | 0.0001-0.1% |
| Minor ingredients (e.g. optical brightener, photo bleach, | 0-5% |
| perfume, suds suppressors) | |

15) A detergent composition formulated as a granulate having a bulk density of at least 600 g/l comprising

| | |
|---|---|
| (C₁₂-C₁₈) alkyl sulfate | 4-8% |
| Alcohol ethoxylate | 11-15% |
| Soap | 1-4% |
| Zeolite MAP or zeolite A | 35-45% |
| Sodium carbonate (as Na₂CO₃) | 2-8% |
| Soluble silicate (as Na₂O, 2SiO₂) | 0-4% |
| Sodium percarbonate | 13-22% |
| TAED | 1-8% |
| Carboxymethyl cellulose | 0-3% |
| Polymers (e.g. polycarboxylates and PVP) | 0-3% |
| Enzymes (calculated as pure enzyme protein) | 0.0001-0.1% |
| Minor ingredients (e.g. optical brightener, phosphonate, | 0-3% |
| perfume) | |

16) Detergent formulations as described in 1)-15) which contain a stabilized or encapsulated peracid, either as an additional component or as a substitute for already specified bleach systems.
17) Detergent compositions as described in 1), 3), 7), 9) and 12) wherein perborate is replaced by percarbonate.
18) Detergent compositions as described in 1), 3), 7), 9), 12), 14) and 15) which additionally contain a manganese catalyst. The manganese catalyst may, e.g., be one of the compounds described in “Efficient manganese catalysts for low-temperature bleaching”, Nature 369, 1994, pp. 637-639.
19) Detergent compositions as described in 1)-18) wherein citrate is fully or partly replaced by one of the following chelants NTA; HEDP, DTPA, MGDA or EDDS
20) Detergent composition formulated as a nonaqueous detergent liquid comprising a liquid nonionic surfactant such as, e.g., linear alkoxylated primary alcohol, a builder system (e.g. phosphate), enzyme and alkali. The detergent may also comprise anionic surfactant and/or a bleach system.
Particular forms of dishwashing detergent compositions within the scope of the invention include:

### 1) Powder Automatic Dishwashing Composition

| | |
|---|---|
| Nonionic surfactant | 0.4-2.5% |
| Sodium metasilicate | 0-20% |
| Sodium disilicate | 3-20% |
| Sodium triphosphate | 20-40% |
| Sodium carbonate | 0-20% |
| Sodium perborate | 2-9% |
| Tetraacetylethylenediamine (TAED) | 1-4% |
| Sodium sulphate | 5-33% |
| Enzymes (calculated as pure enzyme protein) | 0.0001-0.1% |

### 2) Powder Automatic Dishwashing Composition

| | |
|---|---|
| Nonionic surfactant (e.g. alcohol ethoxylate) | 1-2% |
| Sodium disilicate | 2-30% |
| Sodium carbonate | 10-50% |
| Sodium phosphonate | 0-5% |
| Trisodium citrate dihydrate | 9-30% |
| Nitrilotrisodium acetate (NTA) | 0-20% |
| Sodium perborate monohydrate | 5-10% |
| Tetraacetylethylenediamine (TAED) | 1-2% |
| Polyacrylate polymer (e.g. maleic | 6-25% |
| acid/acrylic acid co-polymer) | |
| Enzymes (calculated as pure enzyme protein) | 0.0001-0.1% |
| Perfume | 0.1-0.5% |
| Water | 5-10 |

### 3) Powder Automatic Dishwashing Composition

| | |
|---|---|
| Nonionic surfactant | 0.5-2.0% |
| Sodium disilicate | 25-40% |
| Sodium citrate | 30-55% |
| Sodium carbonate | 0-29% |
| Sodium bicarbonate | 0-20% |
| Sodium perborate monohydrate | 0-15% |
| Tetraacetylethylenediamine (TAED) | 0-6% |
| Maleic acid/acrylic acid copolymer | 0-5% |
| Clay | 1-3% |
| Poly(amino acids) | 0-20% |
| Sodium polyacrylate | 0-8% |
| Enzymes (calculated as pure enzyme protein) | 0.0001-0.1% |

### 4) Powder Automatic Dishwashing Composition

| | |
|---|---|
| Nonionic surfactant | 1-2% |
| Zeolite MAP | 15-42% |
| Sodium disilicate | 30-34% |
| Sodium citrate | 0-12% |
| Sodium carbonate | 0-20% |
| Sodium perborate monohydrate | 7-15% |
| Tetraacetylethylenediamine (TAED) | 0-3% |
| Polymer | 0-4% |
| Maleic acid/acrylic acid copolymer | 0-5% |
| Organic phosphonate | 0-4% |
| Clay | 1-2% |
| Enzymes (calculated as pure enzyme protein) | 0.0001-0.1% |
| Sodium sulphate | Balance |

### 5) Powder Automatic Dishwashing Composition

| | |
|---|---|
| Nonionic surfactant | 1-7% |
| Sodium disilicate | 18-30% |
| Trisodium citrate | 10-24% |
| Sodium carbonate | 12-20% |
| Monopersulphate (2 KHSO₅•KHSO₄•K₂SO₄) | 15-21% |
| Bleach stabilizer | 0.1-2% |
| Maleic acid/acrylic acid copolymer | 0-6% |
| Diethylenetriaminepentaacetate, pentasodium salt | 0-2.5% |
| Enzymes (calculated as pure enzyme protein) | 0.0001-0.1% |
| Sodium sulphate, water | Balance |

6) Powder and Liquid Dishwashing Composition with Cleaning Surfactant System

| | |
|---|---|
| Nonionic surfactant | 0-1.5% |
| Octadecyl dimethylamine N-oxide dihydrate | 0-5% |
| 80:20 wt.C18/C16 blend of octadecyl dimethylamine | 0-4% |
| N-oxide dihydrate and hexadecyldimethyl amine N- | |
| oxide dihydrate | |
| 70:30 wt.C18/C16 blend of octadecyl bis | 0-5% |
| (hydroxyethyl)amine N-oxide anhydrous and | |
| hexadecyl bis | |
| (hydroxyethyl)amine N-oxide anhydrous | |
| C₁₃-C₁₅ alkyl ethoxysulfate with an average degree of | 0-10% |
| ethoxylation of 3 | |
| C₁₂-C₁₅ alkyl ethoxysulfate with an average degree of | 0-5% |
| ethoxylation of 3 | |
| C₁₃-C₁₅ ethoxylated alcohol with an average degree of | 0-5% |
| ethoxylation of 12 | |
| A blend of C₁₂-C₁₅ ethoxylated alcohols with an | 0-6.5% |
| average degree of ethoxylation of 9 | |
| A blend of C₁₃-C₁₅ ethoxylated alcohols with an | 0-4% |
| average degree of ethoxylation of 30 | |
| Sodium disilicate | 0-33% |
| Sodium tripolyphosphate | 0-46% |
| Sodium citrate | 0-28% |
| Citric acid | 0-29% |
| Sodium carbonate | 0-20% |
| Sodium perborate monohydrate | 0-11.5% |
| Tetraacetylethylenediamine (TAED) | 0-4% |
| Maleic acid/acrylic acid copolymer | 0-7.5% |
| Sodium sulphate | 0-12.5% |
| Enzymes (calculated as pure enzyme protein) | 0.0001-0.1% |

### 7) Non-Aqueous Liquid Automatic Dishwashing Composition

| | |
|---|---|
| Liquid nonionic surfactant (e.g. alcohol ethoxylates) | 2.0-10.0% |
| Alkali metal silicate | 3.0-15.0% |
| Alkali metal phosphate | 20.0-40.0% |
| Liquid carrier selected from higher | 25.0-45.0% |
| glycols, polyglycols, polyoxides, glycolethers | |
| Stabilizer (e.g. a partial ester of phosphoric acid and a | 0.5-7.0% |
| C₁₆-C₁₈ alkanol) | |
| Foam suppressor (e.g. silicone) | 0-1.5% |
| Enzymes (calculated as pure enzyme protein) | 0.0001-0.1% |

### 8) Non-Aqueous Liquid Dishwashing Composition

| | |
|---|---|
| Liquid nonionic surfactant (e.g. alcohol ethoxylates) | 2.0-10.0% |
| Sodium silicate | 3.0-15.0% |
| Alkali metal carbonate | 7.0-20.0% |
| Sodium citrate | 0.0-1.5% |
| Stabilizing system (e.g. mixtures of finely divided | 0.5-7.0% |
| silicone and low molecular weight dialkyl polyglycol | |
| ethers) | |
| Low molecule weight polyacrylate polymer | 5.0-15.0% |
| Clay gel thickener (e.g. bentonite) | 0.0-10.0% |
| Hydroxypropyl cellulose polymer | 0.0-0.6% |
| Enzymes (calculated as pure enzyme protein) | 0.0001-0.1% |
| Liquid carrier selected from higher lycols, polyglycols, | Balance |
| polyoxides and glycol ethers | |

### 9) Thixotropic Liquid Automatic Dishwashing Composition

| | |
|---|---|
| C₁₂-C₁₄ fatty acid | 0-0.5% |
| Block co-polymer surfactant | 1.5-15.0% |
| Sodium citrate | 0-12% |
| Sodium tripolyphosphate | 0-15% |
| Sodium carbonate | 0-8% |
| Aluminium tristearate | 0-0.1% |
| Sodium cumene sulphonate | 0-1.7% |
| Polyacrylate thickener | 1.32-2.5% |
| Sodium polyacrylate | 2.4-6.0% |
| Boric acid | 0-4.0% |
| Sodium formate | 0-0.45% |
| Calcium formate | 0-0.2% |
| Sodium n-decydiphenyl oxide disulphonate | 0-4.0% |
| Monoethanol amine (MEA) | 0-1.86% |
| Sodium hydroxide (50%) | 1.9-9.3% |
| 1,2-Propanediol | 0-9.4% |
| Enzymes (calculated as pure enzyme protein) | 0.0001-0.1% |
| Suds suppressor, dye, perfumes, water | Balance |

### 10) Liquid Automatic Dishwashing Composition

| | |
|---|---|
| Alcohol ethoxylate | 0-20% |
| Fatty acid ester sulphonate | 0-30% |
| Sodium dodecyl sulphate | 0-20% |
| Alkyl polyglycoside | 0-21% |
| Oleic acid | 0-10% |
| Sodium disilicate monohydrate | 18-33% |
| Sodium citrate dihydrate | 18-33% |
| Sodium stearate | 0-2.5% |
| Sodium perborate monohydrate | 0-13% |
| Tetraacetylethylenediamine (TAED) | 0-8% |
| Maleic acid/acrylic acid copolymer | 4-8% |
| Enzymes (calculated as pure enzyme protein) | 0.0001-0.1% |

### 11) Liquid Automatic Dishwashing Composition Containing Protected Bleach Particles

| | |
|---|---|
| Sodium silicate | 5-10% |
| Tetrapotassium pyrophosphate | 15-25% |
| Sodium triphosphate | 0-2% |
| Potassium carbonate | 4-8% |
| Protected bleach particles, e.g. chlorine | 5-10% |
| Polymeric thickener | 0.7-1.5% |
| Potassium hydroxide | 0-2% |
| Enzymes (calculated as pure enzyme protein) | 0.0001-0.1% |
| Water | Balance |

12) Automatic dishwashing compositions as described in 1), 2), 3), 4), 6) and 10), wherein perborate is replaced by percarbonate.
13) Automatic dishwashing compositions as described in 1)-6) which additionally contain a manganese catalyst. The manganese catalyst may, e.g., be one of the compounds described in “Efficient manganese catalysts for low-temperature bleaching”, Nature 369, 1994, pp. 637-639.

An alpha-amylase variant of the invention may be incorporated in concentrations conventionally employed in detergents. It is at present contemplated that, in the detergent composition of the invention, the alpha-amylase variant may be added in an amount corresponding to 0.00001-1.2 mg (calculated as pure enzyme protein) of alpha-amylase per liter of wash/dishwash liquor.

### EXAMPLES

### Materials

### Enzymes:

### SP722: SEQ ID NO: 3

### SP707: SEQ ID NO: 4

### AA560: SEQ ID NO: 5

### SP690: SEQ ID NO: 6

### Methods:

### General Molecular Biology Methods:

Unless otherwise mentioned the DNA manipulations and transformations were performed using standard methods of molecular biology (Sambrook et al. (1989); Ausubel et al. (1995); Harwood and Cutting (1990)).

### Fermentation of Alpha-Amylases and Variants

Fermentation may be performed by methods well known in the art or as follows.

A B. subtilis strain harboring the relevant expression plasmid is streaked on a LB-agar plate with a relevant antibiotic, and grown overnight at 37° C.

The colonies are transferred to 100 ml BPX media supplemented with a relevant antibiotic (for instance 10 mg/l chloroamphinicol) in a 500 ml shaking flask.

Composition of BPX medium:

| | |
|---|---|
| Potato starch | 100 g/l |
| Barley flour | 50 g/l |
| BAN 5000 SKB | 0.1 g/l |
| Sodium caseinate | 10 g/l |
| Soy Bean Meal | 20 g/l |
| Na₂HPO₄, 12 H₂O | 9 g/l |
| Pluronic ™ | 0.1 g/l |

The culture is shaken at 37° C. at 270 rpm for 4 to 5 days.

Cells and cell debris are removed from the fermentation broth by centrifugation at 4500 rpm in 20-25 minutes. Afterwards the supernatant is filtered to obtain a completely clear solution. The filtrate is concentrated and washed on an UF-filter (10000 cut off membrane) and the buffer is changed to 20 mM Acetate pH 5.5. The UF-filtrate is applied on a S-sepharose F.F. and elution is carried out by step elution with 0.2 M NaCl in the same buffer. The eluate is dialysed against 10 mM Tris, pH 9.0 and applied on a Q-sepharose F.F. and eluted with a linear gradient from 0-0.3M NaCl over 6 column volumes. The fractions, which contain the activity (measured by the Megazyme assay) are pooled, pH was adjusted to pH 7.5 and remaining color was removed by a treatment with 0.5% W/vol. active coal in 5 minutes. It may further be advantageous to add a further buffer exchange step, e.g. by dialysis or gelfiltration to a buffer system that does not affect the wash result in itself, e.g. to a tris-buffer, an acetate buffer or the like, preferably with a small concentration of calcium (e.g. 0.1 mM) to stabilize the amylase during storage and about 0.01% Triton X-100 to reduce risk of adsorption of enzyme protein to containers and pipettes.

### Assays for Alpha-Amylase Activity

### 1. Megazyme Assay

Alpha-amylase activity is determined by a method employing Amylazyme® tablets as substrate. Amylazyme tablets (Megazyme® Amylazyme Test, supplied by Megazyme for the assay of cereal and bacterial amylases) contain AZCL-amylose, which has been mixed with lactose and magnesium stearate and tabletted.

For every single measurement one tablet is suspended in a tube containing 5 mL 50 mM Britton-Robinson buffer (50 mM acetic acid, 50 mM phosphoric acid, 50 mM boric acid, 0.1 mM CaCl₂, 0.01% Triton X-100, pH adjusted to the value of interest with NaOH). This is the substrate solution. The alpha-amylase to be tested is diluted in×ml of 50 mM Britton-Robinson buffer. This is the amylase solution. The test is performed at 20 deg C. (or 50 deg C.). The AZCL-amylose is hydrolyzed by the alpha-amylase giving soluble blue fragments. The absorbance of the resulting blue solution, measured spectrophotometrically at 650 nm, is a function of the alpha-amylase activity.

575 μL substrate solution is equilibrated at 20 deg C. (or 50 deg C.) for 5 minutes. The hydrolysis is started by adding 25 μL amylase solution to the substrate solution and incubate the sample under gentle mixing for 15 minutes at 20 deg C. (or 50 deg C.). The reaction is stopped by addition of 100 μL 1 M NaOH and immediate cooling on ice-bath after mixing. After centrifugation at 5000 gₐᵥ for 3 minutes, 200 μL of the supernatant is transferred to a microtiter plate, and the absorbance at 650 nm is read (A_{amyl}). The blind is prepared as described but where the 25 μL amylase solution is replaced by 25 μL 50 mM Britton-Robison buffer. The absorbance of the blind at 650 nm is A_{b}. The activity is given as A_{amyl}−A_{b} divided by the concentration of amylase protein in the amylase solution (in mg enzyme protein per mL sample). It is important that the measured 650 nm absorbance after 15 minutes of incubation (testing time) is in the range of 0.2 to 2.0 absorbance units at 650 nm. In this absorbance range there is linearity between activity and absorbance (Lambert-Beer law). The dilution of the enzyme must therefore be adjusted to fit this criterion. Under a specified set of conditions (temperature, pH, reaction time, buffer conditions) 1 mg of a given alpha-amylase will hydrolyze a certain amount of substrate and a blue color will be produced.

### 2. Alternative Assay for Amylase Activity Used for Testing Amylase Stability in Detergent.

Alpha-amylase activity is determined by a method employing the PNP-G7 substrate. PNP-G7 which is a abbreviation for 4,6-ethylidene(G₇)-p-nitrophenyl(G₁)-α,D-maltoheptaoside, a blocked oligosaccharide which can be cleaved by an endo-amylase. Following the cleavage, the alpha-Glucosidase included in the kit digest the substrate to liberate a free PNP molecule which has a yellow color and thus can be measured by visible spectrophotometry at λ=405 nm. (400-420 nm.). Kits containing PNP-G7 substrate and alpha-Glucosidase is manufactured by Roche/Hitachi (cat. No. 11876473). The G7-PNP substrate from this kit contains 22 mmol/L 4,6-ethylidene-G7-PNP and 52.4 mmol/L HEPES (2-[4-(2-hydroxyethyl)-1-piperazinyl]-ethanesulfonic acid), pH 7.0) (working solution) and the alpha-Glucosidase contains 52.4 mmol/L HEPES, 87 mmol/L NaCl, 12.6 mmol/L MgCl₂, 0.075 mmol/L CaCl₂, ≧4 kU/L alpha-glucosidase). The amylase sample to be analysed is diluted in 30 mmol/L CaCl₂, 0.0025% (w/w) Brij-35. The assay is performed by transforming 200 μL alpha-Glucosidase and 16 μL amylase sample solution to a 96 well microtitre plate and incubating at 37 deg C. 20 μl working solution, 37 deg C. is added. The solution is mixed and pre-incubated 1 minute and absorption is measured every 15 sec. over 3 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

In a few instances there is a significant interference from the detergent without amylase on the assay. In such cases alternative amylase assays can be used. Interference from a detergent on an amylase assay can be tested by adding a known amount of amylase to the detergent at two levels and then measure the activity of the two samples. If the difference in the measured activities corresponds to the differences in the levels between the added amylases, the assay can be used to determine the residual activity of the amylase after storage.

### Alpha-Amylase Stability in Detergents

The amylase stability in detergent was tested in a commercial Tide 2× Ultra product or in a model detergent, the composition of which is given below.

Composition of model Detergent:

| | | % active |
|---|---|---|
| Compound | Amount g/100 g | ingredient |
| | Surfactants | |
| Na-LAS (92%) (Nacconol 90G) (anionic) | 10.87 | 10 |
| STEOL CS-370E (70%) (anionic) | 7.14 | 5 |
| Bio-soft N25-7 (99.5%) (non-ionic) | 5 | 5 |
| Oleic acid (fatty acid) | 2 | 2 |
| | Solvents | |
| H₂O | 62 | 65 |
| Ethanol | 0.5 | 0.5 |
| STS (sodium p-toluene sulfonate (40%) | 3.75 | 1.5 |
| Mono propylene glycol | 2 | 2 |
| | Builder | |
| Tri-sodium-citrate | 4 | 4 |
| Triethanolamine (TEA) | 0.5 | 0.5 |
| | Inhibitor | |
| Boric acid | 1.5 | 1.5 |
| | Minors | |
| 10N NaOH (for adjustment to pH 8.5) | 0.8 | 0.8 |

The commercial Tide 2× Ultra were first subjected to a heat treatment (heating at 90° C. for 10 minutes in a microwave) to inactivate the enzymes already present in the product and then cooled to room temperature before use. The model detergent was used directly.

The amylase to be tested was added to the detergent to a final concentration of 0.3 mg amylase protein per mL detergent. After thorough mixing, aliquots of 5 mL were transferred to vials, which were sealed. Two vials of each detergent sample were prepared, one of these was stored at −18° C. for two weeks and one was stored at 37° C. for two weeks. After storage, the residual activity was determined by using the Alternative assay for amylase activity. In some experiments, 26 mM calcium chloride was added to the detergent before addition of enzyme.

It is important that the sample pairs (i.e. the samples stored at 37° C. and −18° C.) are diluted identical to reduce interferences from other detergent components on the assay. The residual activity is calculated as the measured activity in the sample stored 2 weeks at 37° C. multiplied by 100 and divided by the measured activity in the sample stored at −18° C.

### Wash Performance of Alpha-Amylase Using AMSA

In order to assess the wash performance of the alpha-amylase variants in a detergent base composition, washing experiments may be performed. The enzymes are tested using the Automatic Mechanical Stress Assay (AMSA). With the AMSA test the wash performance of a large quantity of small volume enzyme-detergent solutions can be examined. The AMSA plate has a number of slots for test solutions and a lid firmly squeezing the textile swatch to be washed against all the slot openings. During the washing time, the plate, test solutions, textile and lid are vigorously shaken to bring the test solution in contact with the textile and apply mechanical stress in a regular, periodic oscillating manner. For further description see WO 02/42740, especially the paragraph “Special method embodiments” at page 23-24.

### General Wash Performance Description

A test solution comprising water (15° dH), 0.8 g/L detergent, e.g. model detergent B as described below, or 50 mM HCO3-, and the enzyme of the invention, e.g. at concentration of 0, 0.2, 0.4, 0.8 and/or 1.2 mg enzyme protein/L, is prepared. Fabrics stained with starch (e.g. CS-28 from Center For Testmaterials BV, P.O. Box 120, 3133 KT, Vlaardingen, The Netherlands) is added and washed for 30 minutes at 20° C. After thorough rinse under running tap water and drying in the dark, the light intensity or reflectance values of the stained fabrics are subsequently measured as a measure for wash performance. The test with 0 mg enzyme protein/L is used as a blank to obtain a delta remission value. Preferably mechanical action is applied during the wash step, e.g. in the form of shaking, rotating or stirring the wash solution with the fabrics.

The AMSA wash performance experiments were conducted under the experimental conditions specified below:

TABLE A
Experimental condition

| | |
|---|---|
| Detergent | Model detergent B (see below) |
| Detergent dosage | 0.8 g/L |
| Test solution volume | 160 micro L |
| pH | As is |
| Wash time | 30 minutes |
| Temperature | 20° C. |
| Water hardness | 15°dH |
| Enzyme concentration in test solution | 0.2; 0.4; 0.8; 1.2 mg/L |
| Test material | CS-28 (Rice starch cotton) |

TABLE B
Model detergent B

| | Amount | % active |
|---|---|---|
| Compound | g/100 g | ingredient |
| | Surfactants | |
| Na-LAS (92%) (Nacconol 90G) (anionic) | 21.74 | 20 |
| STEOL CS-370E (70%) (anionic) | 14.28 | 10 |
| Bio-soft N25-7 (99.5%) (non-ionic) | 10 | 10 |
| Oleic acid (fatty acid) | 4 | 4 |
| | Solvents | |
| H₂O | 25 | ~33 |
| Ethanol | 0.5 | 0.5 |
| STS (sodium p-toluene sulfonate (40%) | 3.75 | 1.5 |
| Mono propylene glycol | 7 | 7 |
| | Builder | |
| Tri-sodium-citrate | 8 | 8 |
| Triethanolamine (TEA) | 0.5 | 0.5 |
| | Inhibitor | |
| Boric acid | 1.5 | 1.5 |
| | Minors | |
| 10N NaOH (for adjustment to pH 8.5) | 2 | |

Water hardness was adjusted to 15° dH by addition of CaCl2, MgCl2, and NaHCO3 (Ca2+:Mg2++:HCOhd 3⁻=4:1:7.5) to the test system. After washing the textiles were flushed in tap water and dried.

The wash performance is measured as the brightness of the colour of the textile washed. Brightness can also be expressed as the intensity of the light reflected from the sample when illuminated with white light. When the sample is stained the intensity of the reflected light is lower, than that of a clean sample. Therefore the intensity of the reflected light can be used to measure wash performance.

Colour measurements are made with a professional flatbed scanner (Kodak iQsmart, Kodak), which is used to capture an image of the washed textile.

To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}.

### Textiles:

Textile sample CS-28 (rice starch on cotton) is obtained from Center For Testmaterials BV, P.O. Box 120, 3133 KT Vlaardingen, the Netherlands.

Results of the AMSA laundry test of different variants are shown below. In the result the index is 100. The performance result of the parent alpha-amylase is assigned the value of 100 and the results of the variants are compared to this value.

### Wash Performance Test Using Beakers

This assay is a small scale model of a top loaded washing machine and used to evaluate the washing performance of amylases.

The beaker wash performance test, using 250 mL beakers and a paddle stirrer providing oscillating rotational motion, 180° in each direction, with a frequency of 80 per minute, comprises the following steps: providing 100 mL wash solution (6° C., 15° dH, pH 8.0) containing 50 mM NaHCO₃ and 0.4 mg/L enzyme; adding two swatches of CS-28 (5×5 cm) and two swatches of EMPA 162 (5×5 cm) to the wash solution to start the wash; setting the agitation speed to 80 rpm; stopping the agitation after 60 minutes, rinsing the swatches under cold running tap water; drying the rinsed swatches in the dark over night; and evaluating the wash performance by measuring the remission of incident light at 460 nm using Color Eye as described below.

### Equipment and Material

Water bath (5° C.) with circulation; glass beakers (250 mL); one rotating arm per beaker with capacity of 100 mL of washing solution; test swatches: CS-28 (rice starch on cotton) from Center for Testmaterials BV, Vlaardingen, The Netherlands and EMPA 162 (rice starch on cotton/polyester) from EMPA Testmaterials AG, St. Gallen, Switzerland, the swatches are cut into 5×5 cm.

Wash solution: 50 mM NaHCO₃ buffer, pH 8.0, water hardness: 15° dH,

Calcium:Magnesium ratio 4:1.
Amylase stock solution: 1 mg enzyme protein per mL.—A solution of 0.1% (w/v) Triton X-100 and 0.1 mM CaCl₂ in ultrapure water (MilliQ water) is used for dilution of amylase (amylase dilution buffer).

### Color Eye Measurement

Wash performance is expressed as a delta remission value (ΔRem). Light reflectance evaluations of the swatches were done using a Macbeth Color Eye 7000 reflectance spectrophotometer with very small oval aperture, i.e. 0.7 cm²(˜0.7×1.0 cm). The measurements were made without UV in the incident light and remission at 460 nm was extracted. The swatch to be measured was placed on top of another swatch of the same type before being measured to reduce reflection from the piston pushing the swatch up against the measuring opening. Delta remission values for individual swatches were calculated by subtracting the remission value of the swatch washed without added amylase (control) from the remission value of the swatch washed with amylase.

### Example 1

### Preparation of Variants

Using the parent alpha-amylase the following variants were constructed:

The Amylase variants of SEQ ID NO: 3, 4 and 6 were prepared by standard procedures, in brief: Introducing random and/or site-directed mutations into the gene, transforming Bacillus subtilis host cells with the mutated genes, fermenting the transformed host cells (e.g. as described in Example 1 of WO 2004/111220), and purifying the protease from the fermentation broth. The reference amylases (e.g. the parent) SEQ ID NO: 3, 4 and 6 respectively were produced recombinantly in Bacillus subtilis in a similar manner.

### Example 2

### Activity at Low Temperature

The activities of the variant amylases were tested as described in the Methods section using the Megazyme assay. The activity was compared to the activity of the parent amylase. The activity was determined at pH 8 and 20° C.

### Results:

The activity of the amylase variants relative to the activity of the parent amylase is shown in the table. The activity of the parent amylase is set to index 100.

TABLE 2.1
Alpha-amylase activities

| | | Activity relative | | Activity relative | |
|---|---|---|---|---|---|
| | | to parent | | to parent | |
| | | amylase, 20° C. | | amylase, 50° C. | |
| Amylase | | pH 8 | pH 10 | pH 8 | pH 10 |
| Parent | SP690 | 100 | 100 | 100 | 100 |
| Variant A of | SP690 + | 134 | 158 | 110 | 93 |
| SP690 | T183* + G184* | | | | |
| Variant B of | SP690 + | 146 | 151 | 109 | 67 |
| SP690 | R181* + G182* | | | | |
| Parent | SP722 | 100 | 100 | 100 | 100 |
| Variant C of | SP722 + | 197 | 213 | 116 | 99 |
| SP722 | D183* + G184* | | | | |
| Parent | SP707 | 100 | 100 | 100 | 100 |
| Variant D of | SP707 + | 160 | 242 | 120 | 122 |
| SP707 | H183* + G184* | | | | |

The results clearly demonstrate the increased activity of the variants a low temperature relative to the corresponding parent amylase. It can also be seen that the variants maintains a significant activity at 50° C. relative to the corresponding parent amylase.

### Example 3

### Stability in Detergent

The amylase stability in detergent was tested with SP707 and the variant SP707, G182*, H183* in two detergents, a commercial Tide 2× Ultra and a model detergent as described in the Methods section.

### Results

The residual activity of amylase after storage at 2 weeks at 37° C. relative to residual activity after storage for 2 weeks at −18° C.

TABLE 3.1
Residual alpha-amylase activity

| | | Tide Ultra 2X | | Model detergent | |
|---|---|---|---|---|---|
| Amylase | | | +Calcium | | +Calcium |
| Parent | SP707 | 4% | 41% | 0% | nd |
| Variant D | SP707 + | 93% | 94% | 45% | nd |
| of SP707 | H183* + G184* | | | | |

The residual stability of the variant is much improved compared to the stability of the parent amylase. Addition of calcium to the detergent during storage stabilizes the parent amylase to some extent, indicating that the low stability of the parent amylase is explained by sensitivity to a low calcium environment, e.g. presence of a builder.

### Example 4

### Wash Performance

The wash performance of the variants and corresponding parent amylases were tested at 20° C. by the AMSA-test method as described in the Methods section using model detergent B.

### Results:

The relative wash performance at different amylase dosages are shown in the table below.

TABLE 4.1
Relative wash performance

| | | Relative wash performance | | | |
|---|---|---|---|---|---|
| | | Amylase dosage (mg enzyme | | | |
| | | protein per L wash liquor) | | | |
| Amylase | | 0.2 | 0.4 | 0.8 | 1.2 |
| Parent | SP690 | 100 | 100 | 100 | 100 |
| Variant A of | SP690 + T183* + G184* | 135 | 119 | 131 | 120 |
| SP690 | | | | | |
| Variant B of | SP690 + R181* + G182* | 149 | 156 | 139 | 130 |
| SP690 | | | | | |
| Parent | SP722 | 100 | 100 | 100 | 100 |
| Variant C of | SP722 + D183* + G184* | 195 | 152 | 143 | 127 |
| SP722 | | | | | |
| Parent | SP707 | 100 | 100 | 100 | 100 |
| Variant D of | SP707 + H183* + G184* | 248 | 179 | 219 | 174 |
| SP707 | | | | | |

The wash test results clearly demonstrate the increased wash performances of the variant amylases, relative to the corresponding parent amylases, at low temperature.

### Example 5

### Wash Performance of Alpha-Amylases at 15° C., 30° C. and 35° C. In Model Detergent

The wash performance of the variants and corresponding parent alpha-amylases were tested by the AMSA-test method as described in the Methods section using model detergent B, with the following modifications with respect to temperatures in the wash and washing time: 15° C. for 45 minutes, 30° C. for 20 minutes and 35° C. for 20 minutes. Furthermore only one enzyme dosage was used, 0.4 mg enzyme protein per L wash solution. Each enzyme candidate was tested twice. The results are given as (performance of variant minus performance of blank) divided by (performance of parent minus performance of blank) multiplied by 100, where the blank is the performance obtained by washing at the same conditions, but in the absence of alpha-amylase.

The relative wash performance at different amylase dosages are shown in the table below.

TABLE 5.1
Relative wash performance at wash temperatures
15° C., 30° C. and 35° C.

| | | 45 min. | 20 min. | 20 min. |
|---|---|---|---|---|
| | | 15° C. | 30° C. | 35° C. |
| Parent | SP690 | 100 | 100 | 100 |
| Variant A of SP690 | SP690 + T183* + | 131 | 160 | 137 |
| | G184* | | | |
| Variant B of SP690 | SP690 + R181* + | 130 | 173 | 157 |
| | G182* | | | |
| Parent | SP722 | 100 | 100 | 100 |
| Variant C of SP722 | SP722 + D183* + | 163 | 154 | 158 |
| | G184* | | | |
| Parent | SP707 | 100 | 100 | 100 |
| Variant D of SP707 | SP707 + H183* + | 272 | 302 | 277 |
| | G184* | | | |
| Parent A | AA560 | 100 | 100 | 100 |
| Parent B | AA560 + R118K + | 96 | 91 | 100 |
| | N195F + R320K, | | | |
| | R458K | | | |
| Variant of Parent A | AA560 + R118K + | 121 | 148 | 146 |
| and Parent B | D183* + G184* + | | | |
| | N195F + R320K + | | | |
| | R458K | | | |

The wash performance test clearly demonstrates that the performances of the variants are much improved relative to their respective parent molecules at all the temperatures tested.

TABLE 5.2
Wash performance at 15° C., 30° C. and 35° C. relative to SP707

| | | 45 min. | 20 min. | 20 min. |
|---|---|---|---|---|
| | | 15° C. | 30° C. | 35° C. |
| SP707 | | 100 | 100 | 100 |
| Variant A | SP690 + T183* + G184* | 234 | 238 | 137 |
| of SP690 | | | | |
| Variant B | SP690 + R181* + G182* | 233 | 256 | 157 |
| of SP690 | | | | |
| Variant C | SP722 + D183* + G184* | 349 | 356 | 158 |
| of SP722 | | | | |
| Variant D | AA560 + R118K + D183* + | 247 | 328 | 146 |
| of AA560 | G184* + N195F + R320K + | | | |
| | R458K | | | |

The wash performance test also clearly demonstrates that the performances of the variants are much improved relative to the performance of SP707 (SEQ ID NO 4).

### Example 6

### Wash Performance of Alpha-Amylase AA560 and Variants Thereof in Model Detergent

The wash performance of AA560 and AA560 variants was tested using the AMSA-test method as described in the Methods section using model detergent B at four different amylase concentrations at a washing temperature of 20° C. Each amylase was tested four times at each concentration and the experiment was repeated twice.

The results are given as (performance of variant minus performance of blank) divided by (performance of parent minus performance of blank) multiplied by 100, where the blank is the performance obtained by washing at the same conditions, but in the absence of amylase, and then averaged.

TABLE 6.1
Relative wash performance at wash temperature 20° C.

| | | Amylase dosage | | | |
|---|---|---|---|---|---|
| | | (mg enzyme protein per L wash liquor) | | | |
| | | 0.2 | 0.4 | 0.8 | 1.2 |
| | | Relative wash performance (%) | | | |
| Parent A | AA560 | 100 | 100 | 100 | 100 |
| Parent B | AA560 + R118K + | 104 | 103 | 115 | 105 |
| | N195F + R320K + | | | | |
| | R458K | | | | |
| Variant | AA560 + R118K + | 151 | 156 | 153 | 145 |
| of Parent | D183* + G184* + | | | | |
| A and | N195F + R320K + | | | | |
| Parent B | R458K | | | | |

It can clearly be seen that the variant having the deletions at position D183 and G184 are significantly improved relative to the alpha-amylase Parent A and Parent B when tested at 20° C.

### Example 7

### Wash Performance of Alpha-Amylase SP722 and Variants Thereof in Model Detergent

The wash performance of SP722 and SP722 variants was tested using the AMSA-test method as described in the Methods section using model detergent B at four different amylase concentrations at a washing temperature of 20° C. Each amylase was tested four times at each concentration and the experiment was repeated twice.

The results are given as (performance of variant minus performance of blank) divided by (performance of parent minus performance of blank) multiplied by 100, where the blank is the performance obtained by washing at same conditions, but in the absence of amylase, and then averaged.

The wash performances of different single and double deletion variants of SP722 relative to the wash performance of SP722 are presented in the table below.

TABLE 7.1
Relative wash performance with difference dosages at 20° C.

| | | Relative performance (%) | | | |
|---|---|---|---|---|---|
| | | Amylase dosage (mg enzyme protein per L of wash liquor) | | | |
| | | 0.2 mg/L | 0.4 mg/L | 0.8 mg/L | 1.2 mg/L |
| Parent | SP722 | 100 | 100 | 100 | 100 |
| Variant A of | SP722 + R181* + | 190 | 154 | 139 | 141 |
| SP722 | G182* | | | | |
| Variant B of | SP722 + R181* | 171 | 140 | 138 | 131 |
| SP722 | | | | | |
| Variant C of | SP722 + D183* | 159 | 143 | 125 | 135 |
| SP722 | | | | | |
| Variant D of | SP722 + G182* | 152 | 128 | 129 | 126 |
| SP722 | | | | | |
| Variant E of | SP722 + G184* | 173 | 151 | 148 | 133 |
| SP722 | | | | | |
| Variant F of | SP722 + R181* + | 167 | 134 | 148 | 131 |
| SP722 | G182* + D183G | | | | |

The wash performance of both the single deletion and the double deletion variants are much improved relative to the parent amylase, SP722, when tested at 20° C.

### Example 8

### Wash Performance of AA560 and AA560 Variants in Bicarbonate Buffer pH 8

The wash performance of alpha-amylase AA560 and AA560 variants was tested using the AMSA-test method as described in the Methods section using 50 mM HCO₃ buffer as wash solution at four different amylase concentrations at a washing temperature of 20° C. Each amylase was tested four times at each concentration and the experiment was repeated twice.

The results are given as (performance of variant minus performance of blank) divided by (performance of parent minus performance of blank) multiplied by 100, where the blank is the performance obtained by washing at same conditions, but in the absence of amylase, and then averaged.

The results are presented in the table below.

TABLE 8.1
Relative wash performance with different dosages at 20° C.

| | | Amylase dosage | | | |
|---|---|---|---|---|---|
| | | (mg enzyme protein per L wash liquor) | | | |
| | | 0.2 | 0.4 | 0.8 | 1.2 |
| | | Relative wash performance (%) | | | |
| Parent A | AA560 | 100 | 100 | 100 | 100 |
| Parent B | AA560 + R118K + | 98 | 106 | 101 | 102 |
| | N195F + R320K + | | | | |
| | R458K | | | | |
| Variant | AA560 + R118K + | 171 | 165 | 137 | 128 |
| of Parent | D183* + G184* + | | | | |
| A and | N195F + R320K + | | | | |
| Parent B | R458K | | | | |

It can clearly be seen that the wash performance of the alpha-amylase variant having the deletions at position D183 and G184 are significantly improved relative to the Parent A and Parent B when tested at 20° C. in an AMSA wash performance test with bicarbonate buffer.

### Example 9

### Wash Performance of SP722 and SP722 Variants in Bicarbonate Buffer

The wash performance of alpha-amylase SP722 and SP722 variants was tested using the AMSA-test method as described in the Methods section using 50 mM HCO₃ buffer as wash solution) at four different amylase concentrations at 20° C. Each amylase was tested four times at each concentration and the experiment was repeated twice.

The results are given as (performance of variant minus performance of blank) divided by (performance of parent minus performance of blank) multiplied by 100, where the blank is the performance obtained by washing at same conditions but in the absence of amylase and then averaged.

The wash performances of different single and double deletion variants of SP722 relative to the wash performance of SP722 are presented in the table below.

TABLE 9.1
Relative wash performance with different dosages at 20° C.

| | | Relative wash performance (%) | | | |
|---|---|---|---|---|---|
| | | Amylase dosage (mg enzyme protein per L of wash liquor) | | | |
| | | 0.2 mg/L | 0.4 mg/L | 0.8 mg/L | 1.2 mg/L |
| Parent | SP722 | 100 | 100 | 100 | 100 |
| Variant A of | SP722 + R181* + | 163 | 152 | 128 | 129 |
| SP722 | G182* | | | | |
| Variant B of | SP722 + R181* | 153 | 140 | 123 | 125 |
| SP722 | | | | | |
| Variant C of | SP722 + D183* | 143 | 143 | 121 | 124 |
| SP722 | | | | | |
| Variant D of | SP722 + G182* | 142 | 148 | 126 | 127 |
| SP722 | | | | | |
| Variant E of | SP722 + G184* | 165 | 155 | 129 | 117 |
| SP722 | | | | | |
| Variant F of | SP722 + R181* + | 164 | 149 | 121 | 125 |
| SP722 | G182* + D183G | | | | |

The wash performance of both the single deletion and the double deletion variants are much improved relative to the parent amylase, SP722, when tested at 20° C.

### Example 10

### Stability of Alpha-Amylase in Detergents with or without Addition of Calcium

The amylase stability in detergent was tested with SP722 and variants of SP722 having a double deletion in a model detergent as described in the Methods section.

The residual activity of the alpha-amylase was determined after 2 weeks storage at 37° C. relative to residual activity after storage for 2 weeks at −18° C.

The results are shown in Table 10.1.

TABLE 10.1
Residual activity
Relative residual activity (%) after 2 weeks at 37° C.

| | No addition | Addition |
|---|---|---|
| | of calcium | of calcium |
| SP722 | 0.1 | 94 |
| SP722 + 181* + 182* | 45 | 88 |
| SP722 + 181* | 1.1 | 60 |
| SP722 + 182* | 0.1 | 78 |
| SP722 + 183* | 0.2 | 58 |
| SP722 + 184* | 0 | 56 |
| SP722 + 181* + 182 + D183G | 47 | 94 |

The results clearly demonstrate that the stability of double deletion alpha-amylase variants in liquid detergent is improved. Addition of calcium to the detergent during storage stabilizes both parent and variant amylase to some extent, indicating that the stability of the amylase is explained by sensitivity to a low calcium environment, e.g. presence of a builder.

### Example 11

### Stability of Alpha-Amylase in Detergents with or without Addition of Calcium

The amylase stability in detergent was tested with AA560 and two variants thereof in a model detergent as described in the Methods section.

The residual activity of alpha-amylase after 2 weeks storage at 37° C. relative to residual activity after storage for 2 weeks at −18° C.

TABLE 11.1
Relative residual activity after storage at 37° C.
Relative residual activity (%) after 2 weeks storage at 37° C.

| | No addition | +Addition |
|---|---|---|
| | of calcium | of calcium |
| AA560 | 0.4 | 69 |
| AA560 + R118K + N195F + | 8 | 85 |
| R320K + R458K | | |
| AA560 + R118K + D183* + | 67 | 115 |
| G184* + N195F + R320K + | | |
| R458K | | |

These data clearly demonstrates that the D183*, G184* double deletion improves the storage stability in liquid detergent significantly. Addition of calcium to the detergent during storage stabilizes both parent and variant amylase to some extent, indicating that the stability of the amylase is explained by sensitivity to a low calcium environment, e.g. presence of a builder.

### Example 12

### Wash Performance Test in Beakers

A series of different alpha-amylases and variants thereof were tested for their wash performance using the wash performance test using beakers described in the above Methods section.

The washing conditions were the following:

| | |
|---|---|
| Washing solution | 50 mM NaHCO₃, pH 8.0 (pH is checked after |
| | addition of water hardness ions and re- |
| | adjusted if necessary) |
| Water hardness | 15° dH (Ca²⁺:Mg²⁺ 4:1) |
| Temperature | 6° C. in washing solution |
| Amylase concentration | 0.4 mg enzyme protein/L wash solution |
| in test solution | |
| Test material | CS-28 and EMPA 162 |
| Washing time | 60 minutes |

Each beaker contained two swatches of each type, and three beakers of each amylase were run in parallel. The whole experiment was repeated twice and the results presented are an average of the obtained readings for each alpha-amylase.

As described in the Methods section, the delta remissions of the swatches are determined after wash and the results are given as the delta remission of the variant relative to the delta remission of the corresponding parent amylase. The results are presented in the table below.

TABLE 12.1
Wash performance at 6° C.

| | | | | Performance | | Performance | |
|---|---|---|---|---|---|---|---|
| | | | | (%) | | (%) | |
| | | Delta | | relative to | | relative to | |
| | | remission | | parent | | SP707 | |
| | | | EMPA | | EMPA | | EMPA |
| | | CS-28 | 162 | CS-28 | 162 | CS-28 | 162 |
| Parent | SP690 | 2.45 | 4.07 | 100 | 100 | — | — |
| Variant A of | SP690 + T183* + G184* | 2.86 | 4.31 | 116 | 106 | 113 | 124 |
| SP690 | | | | | | | |
| Variant B of | SP690 + R181* + G182* | 3.93 | 5.73 | 162 | 141 | 155 | 165 |
| SP690 | | | | | | | |
| Parent | SP722 | 2.99 | 4.09 | 100 | 100 | — | — |
| Variant C | SP722 + D183* + G184* | 5.65 | 7.20 | 202 | 176 | 223 | 207 |
| of SP722 | | | | | | | |
| Parent A | AA560 | 3.55 | 4.54 | 100 | 100 | — | — |
| Parent B | AA560 + | 5.72 | 5.38 | 176 | 119 | — | — |
| | R118K + N195F + | | | | | | |
| | R320K + R458K | | | | | | |
| Variant of | AA560 + R118K + D183*, | 6.72 | 10.70 | 192 | 236 | 266 | 308 |
| Parent A | G184* + N195F + R320K + | | | | | | |
| and Parent B | R458K | | | | | | |
| Parent | SP707 | 2.53 | 3.47 | 100 | 100 | 100 | 100 |
| Variant D | SP707 + H183* + G184* | 7.02 | 9.87 | 291 | 294 | 278 | 284 |
| of SP707 | | | | | | | |

The results presented in table 12.1 clearly demonstrate that introduction of a double deletion in SP690, SP722, AA560 or SP707 significantly improves the wash performance at 6° C. relative to the parent of the variant, but also relative to SP707 (SEQ ID NO 4).

### Example 13

### Alpha-Amylase Activity of Alpha-Amylase SP722 and Variants Thereof at Low Temperature

The alpha-amylase activities of variant amylases were tested as described in the Methods section using the Megazyme assay. The activity was compared with the activity of the parent amylase. The activity was determined at pH 8 and pH 10 at 20° C. and 50° C. and the results are given relative to the parent amylase activity (table 13.1) and relative to mg enzyme protein (A_{650 nm} per mg enzyme protein) (table 13.2).

TABLE 13.1
Alpha-amylase activities relative to parent amylase activity

| | |
|---|---|
| pH 8 | pH 10 |

| | 20° C. | 50° C. | 20° C. | 50° C. |
|---|---|---|---|---|
| SP722 (parent) | 100 | 100 | 100 | 100 |
| SP722 + R181* + | 180 | 49 | 128 | 57 |
| G182* | | | | |
| SP722 + R181* | 175 | 35 | 133 | 46 |
| SP722 + G182* | 156 | 44 | 145 | 74 |
| SP722 + D183* | 177 | 42 | 142 | 51 |
| SP722 + G184* | 176 | 48 | 154 | 56 |
| SP722 + R181* + | 208 | 55 | 111 | 61 |
| G182* + D183G | | | | |

The results clearly show that deletions in positions 181, 182, 183 or 184 have a significant positive effect on the alpha-amylase activity at low temperature (20° C.).

TABLE 13.2
Alpha-amylase activity

| | pH 8 | | pH 10 | |
|---|---|---|---|---|
| | 20° C. | 50° C. | 20° C. | 50° C. |
| | A650/mg ep | | | |
| SP722 | 507 | 2572 | 1325 | 1749 |
| SP722 R181*, G182* | 913 | 1265 | 1696 | 989 |
| SP722 R181* | 887 | 901 | 1762 | 811 |
| SP722 G182* | 789 | 1125 | 1927 | 1298 |
| SP722 D183* | 895 | 1085 | 1878 | 888 |
| SP722 G184* | 892 | 1235 | 2043 | 975 |
| SP722 R181*, G182*, D183G | 1056 | 1405 | 1471 | 1066 |

The results demonstrates that apart from having improved activity at 20° C. relative to the parent as discussed above, the variants also have a higher activity at 50° C. at pH 8 than at 20° C., although the activity at the higher temperature is not as high as that of the parent. Whereas at pH 10 and 50° C. these variants show a slight decrease in activity relative to the activity at 20° C.

### Example 14

### Alpha-Amylase Activity of Alpha-Amylase AA560 and Variants Thereof at Low Temperature

The activities of variant amylases were tested as described in the Methods section using the Megazyme assay. The activity was compared with the activity of the parent amylase. The activity was determined at pH 8 and pH 10 both at 20° C. and 50° C. and the results are given relative to the parent alpha-amylase activity (Table 14.1) and activity per mg enzyme protein, calculated as A_{650 nm} per mg enzyme protein (Table 14.2).

TABLE 14.1
Relative alpha-amylase activities

| | pH 8 | pH 10 |
|---|---|---|
| | 20° C. | 20° C. |
| AA560 | 100 | 100 |
| AA560 + R118K + N195F + | 93 | 104 |
| R320K + R458K | | |
| AA560 + R118K + D183* + | 103 | 128 |
| G184* + N195F + R320K + | | |
| R458K | | |

The data clearly shows that the double deletion of D183* and G184* increases the activity of the amylase at low temperature relative to the parent (both Parent A and Parent B).

TABLE 14.2
Alpha-amylase activity

| | pH 8 | | pH 10 | |
|---|---|---|---|---|
| | 20° C. | 50° C. | 20° C. | 50° C. |
| AA560 | 1378 | 12399 | 1039 | 6319 |
| AA560 + R118K + | 1416 | 8991 | 931 | 4902 |
| N195F + R320K + R458K | | | | |
| AA560 + R118K + | 1555 | 7588 | 1215 | 4435 |
| D183* + G184* + | | | | |
| N195F + R320K + R458K | | | | |

The data demonstrates that even though the activity of the variant having a double deletion at position D183*+G184* has a lower activity at 50° C. than the activity of the corresponding parent, the activity at 50° C. is significantly higher than at 20° C., meaning that the variant will perform both at low and high temperature.

### Example 15

### Alpha-Amylase Activity at Low Temperature

The activities of variant amylases were tested as described in the Methods section using the Megazyme assay. The activity was compared with the activity of the parent amylase. The activity was determined at pH 8 at 20° C. and the results are given relative to the parent amylase activity.

TABLE 15.1
Alpha-amylase activities

| | | Relative activity (%) |
|---|---|---|
| | | 20° C., pH 8 |
| Parent | SP707 | 100 |
| Variant A | SP707 + R181* | 130 |
| of SP707 | | |
| Variant B | SP707 + R181* + G182* | 161 |
| of SP707 | | |
| Variant C | SP707 + R181* + G182* + | 184 |
| of SP707 | H183G | |
| Variant D | SP707 + G184* | 110 |
| of SP707 | | |
| Variant E | SP707 + R181* + G184* | 177 |
| of SP707 | | |
| Variant F | SP707 + H183* | 139 |
| of SP707 | | |
| Variant G | SP707 + G182* + H183* | 111 |
| of SP707 | | |

The results clearly show that deletions in positions 181, 182, 183 or 184 have a significant positive effect on the alpha-amylase activity at low temperature (20° C.).

### Example 16

### Specific Alpha-Amylase Activity of Alpha-Amylase AA560 and Variants Thereof

The specific activity for a number of alpha-amylase variants was determined using the PNP-G7 assay as described in the Methods section. The protein concentration was determined by measurement of the absorbance at 280 nm and the theoretical extinction coefficient calculated from the amino acid sequence of the alpha-amylase variant. The specific activity is calculated as the activity divided by the protein concentration, and the results are presented relative to the specific activity of the parent alpha-amylase.

TABLE 16.1
Relative specific activity

| | | Relative specific |
|---|---|---|
| | | activity (%) |
| Parent A | AA560 | 100 |
| Parent B | AA560 + R118K + N195F + | 86 |
| | R320K + R458K | |
| Variant of Parent | AA560 + R118K + D183* + | 99 |
| A and Parent B | G184* + N195F + R320K + | |
| | R458K | |

The results show that the introduction of the double deletion of D183* and G184* does not adversely affect the specific alpha-amylase activity of the variant.

### Example 17

### Specific Alpha-Amylase Activity of Alpha-Amylase SP722 and Variants Thereof

The specific activity for a number of SP722 variants was determined using the PNP-G7 assay as described in the Methods section. The protein concentration was determined by measurement of the absorbance at 280 nm, and the theoretical extinction coefficient calculated from the amino acid sequence of the alpha-amylase variant. The specific activity is calculated as the activity divided by the protein concentration, and the results are presented relative to the specific activity of the parent alpha-amylase.

TABLE 17.1
Relative specific activity

| | Relative specific activity (%) |
|---|---|
| SP722 (parent) | 100 |
| SP722 + R181* + G182* | 94 |
| SP722 + R181* | 101 |
| SP722 + G182* | 109 |
| SP722 + D183* | 104 |
| SP722 + G184* | 112 |
| SP722 + R181* + G182* + D183G | 68 |

The results show that the introduction of the single or double deletions at positions 181, 182, 183 or 184 has limited effect on the specific activity of the alpha-amylase variants relative to the parent alpha-amylase.

### REFERENCES

Ausubel et al., 1995, Short Protocols in Molecular Biology, 3^{rd} Edition, John Wiley and Sons; Caruthers, 1981 “Deoxynucleoside phosphoramidites—A new class of key intermediates for deoxypolynucleotide synthesis”. Tetrahedron Letters 22: 1859;
Feller et al., 2003, Psychrophilic enzymes: hot topics in cold adaptation, Nature Reviews—Microbiology, vol. 1, pp. 201-208;
Georlette, et al. 2004, Some like it cold: biocatalysis at low temperatures. FEMS Microbiology Reviews 28, 25-42;
Harwood and Cutting, 1990 Molecular Biological Methods for Bacillus. In eds. (Chichester, England: John Wiley & Sons);
Hudson et al., 1989, Practical Immunology, Third edition, Blackwell Scientific Publications;
Igarashi et al., 1998, Biochem. Biophys. Res. Comm. 248:772;
Larkin et al., 2007, Bioinformatics 23: 2947-2948;
Matthes et al., 1984, EMBO J. 3 801-805;
Morinaga et al., 1984, Improvement of oligonucleotide-directed site-specific mutagenesis using double-stranded plasmid DNA, Biotechnology 2, pp. 636-639;
Nagarajan et al., 1984, JAOCS, Vol. 61, no. 9, pp. 1475-1478;
Needleman and Wunsch, 1970, J. Mol. Biol. 48, p. 443-453;
Nelson and Long, 1989, A general method of site-specific mutagenesis using a modification of the Thermus aquaticus polymerase chain reaction, Anal. Biochem. 180, pp. 147-151;
Saiki et al., 1988, Primer-directed enzymatic amplification of DNA with a thermostable DNA polymerase, Science, 1988, Vol. 239 no. 4839 pp. 487-491;
Sambrook et al., 1989, Molecular Cloning: A Laboratory Manual, 2nd Ed., Cold Spring Harbor;
Shiau et al., 2003, Appl. Environ. Micro. 69:2383;
Suzuki et al., 1989, J. Biol. Chem. 264(32) 18933-18938;
Taylor, 1986, Journal of Theoretical Biology, 119: 205-218;
Tsukamoto et al. 1988, Biochemical and Biophysical Research Communications, 151, pp. 25-31;
Rice et al., 2000, Trends Genet. 16: 276-277.

1. Use in a starch removing process of a variant or a variant of a parent alpha-amylase, wherein said variant comprises a deletion at one or more positions corresponding to positions selected from the group consisting of 180, 181, 182, 183 and 184 of the mature polypeptide of SEQ ID NO 3, wherein said variant having at least one improved property compared to an alpha-amylase having the identical amino acid sequence of said variant but not having the deletion at one or more of said positions, or compared to the parent alpha-amylase or compared to an alpha-amylase having the amino acid sequence shown in SEQ ID NO 4 and wherein the temperature in the starch removing process is below 40 deg C., such as below 35 deg C.

2. Use in laundry, dish wash, industrial or institutional cleaning of a variant or a variant of a parent alpha amylase, comprising a deletion at one or more positions corresponding to positions selected from the group comprising 180, 181, 182, 183 and 184 of the mature polypeptide of SEQ ID NO 3, wherein said variant having at least one improved property compared to an alpha-amylase having the identical amino acid sequence of said variant but not having the deletion at one or more of said positions, or compared to the parent alpha amylase or compared to SEQ ID NO 4 and wherein the temperature in laundry, dish wash, industrial and institutional cleaning is below 40 deg C., such as below 35 deg C.

3. The use according to claim 1, wherein the starch removing process or the cleaning process is performed at temperature below 32 deg C., preferably below 30 deg C., preferably below 20 deg C., preferably below 10 deg C. or preferably below 5 deg C.

4. The use according to claim 1, wherein the variant has an increased wash performance at 20 deg C. of at least 10% relative to the parent alpha-amylase, or relative to an alpha-amylase having the identical amino acid sequence of said variant but not having the deletion at one or more of said positions or relative to an alpha-amylase having the amino acid sequence shown in SEQ ID NO 4.

5. The use according to claim 1, wherein the activity of the amylase variant at 20 deg C. and at pH 8 is at least 30% increased relative to the parent alpha-amylase, relative to an alpha-amylase having the identical amino acid sequence of said variant but not having the deletion at one or more of said positions or relative to an alpha-amylase having the amino acid sequence shown in SEQ ID NO 4 when measured in the Megazyme assay.

6. The use according to claim 1, wherein the residual activity of the variant is at least 40% after 2 weeks at 37 □Deg C.

7. The use of a variant according to claim 1, wherein the variant comprises a combination of deletions at two or more positions corresponding to positions 180, 181, 182, 183, 184 of the mature polypeptide of SEQ ID NO: 3.

8. The use of a variant according to claim 7, wherein the variant comprises a combination of deletions at two or more positions corresponding to positions 181*+182*; 181*+183*, 182*+183*, 181*+184*, 182*+184* or 183*+184* of the mature polypeptide of SEQ ID NO 3.

9. The use of a variant according to claim 1, wherein the deletions are, or are equivalent to R181*+G182*; R181*+H183*; G182*+H183*; 181*+184*, 182*+184* or H183*+G184 of SEQ ID NO 4.

10. The use according to claim 1, wherein the variant further comprises an mutation at a position corresponding positions 28, 36, 40, 75, 83, 90, 91, 94, 118, 132, 142, 154, 172, 186, 195, 280, 311, 320, 323, 383, 400, 437, 452, 458 and 459 of the mature polypeptide of SEQ ID NO 3.

11. A composition comprising a surfactant and a variant or a variant of a parent alpha-amylase, wherein the variant comprises at least one deletion at one or more positions corresponding to positions selected from the group consisting of 180, 181, 182, 183 and 184 of the mature polypeptide of SEQ ID NO: 3, wherein the variant has at least one improved property relative to an alpha-amylase having the identical amino acid sequence of said variant but not having the deletion at one or more of said positions, or relative to the parent alpha-amylase or relative to an alpha-amylase having the amino acid sequence shown in SEQ ID NO 4 selected from the group consisting of improved activity, improved wash performance and improved stability.

12. The composition according to claim 11, wherein the variant has an increased wash performance at 20 deg C. of at least 10% relative an alpha-amylase having the identical amino acid sequence of said variant but not having the deletion at one or more of said positions, or relative to the parent alpha-amylase or to an alpha-amylase having the amino acid sequence shown in SEQ ID NO 4.

13. The composition according to claim 11, wherein the activity of the amylase variant at 20 deg C. and at pH 8 is at least 30% increased relative to an alpha-amylase having the identical amino acid sequence of said variant but not having the deletion at one or more of said positions, or relative to the parent alpha-amylase or relative to an alpha-amylase having the amino acid sequence shown in SEQ ID NO 4 when measured in the Megazyme assay.

14. The composition according to claim 11, wherein the residual activity of the variant is at least 40% after 2 weeks at 37 □Deg C.

15. A method of cleaning comprising adding to a cleaning process a composition comprising at least one surfactant and a variant alpha-amylase, wherein the variant comprise a deletion at one or more positions corresponding to positions selected from the group consisting of 180, 181, 182, 183 and 184 (using the mature polypeptide of SEQ ID NO 3 for numbering), and wherein said variant comprises an amino acid sequence having at least 60% identity to amino acid sequence from the group consisting of SEQ ID NO 1, SEQ ID NO 2, SEQ ID NO 3, SEQ ID NO 4, SEQ ID NO 5 and SEQ ID NO 6, SEQ ID NO 7, SEQ ID NO 8, SEQ ID NO 9, SEQ ID NO 10, and SEQ ID NO 11, and wherein the variant has at least one improved property relative to an alpha-amylase having the identical amino acid sequence of said variant but not having the deletion at one or more of said positions or relative to an alpha-amylase having the amino acid sequence shown in SEQ ID NO 4 and wherein said cleaning process is performed at a temperature below 40 deg C., such as below 35 deg C.

16. The method of claim 15, wherein said cleaning process is selected from the group consisting of at least one cleaning step in a laundry, dish wash, industrial or institutional cleaning process.

17. The method according to claim 15, wherein the variant has an increased wash performance at 20 deg C. of at least 10% relative to the parent alpha-amylase or to an alpha-amylase having the amino acid sequence shown in SEQ ID NO 4.

18. The method according to claim 15, wherein the activity of the amylase variant at 20 deg C. and at pH 8 is at least 30% increased relative to the parent alpha-amylase or relative to an alpha-amylase having the amino acid sequence shown in SEQ ID NO 4 when measured in the Megazyme assay.

19. The method according to claim 15, wherein the residual activity of the variant is at least 40% after 2 weeks at 37 □Deg C.

20. An article of manufacture comprising
(a) a container holding a cleaning composition comprising a surfactant and an alpha-amylase variant, wherein said variant comprises a deletion at one or more positions corresponding to positions selected from the group consisting of 180, 181, 182, 183 and 184 of the mature polypeptide of SEQ ID NO 3,
(b) published material associated with the container informing that the cleaning composition is useful in a cleaning process which is performed at a temperature below 40, such as below 35 deg. C.
